(19) United States
(12) Patent Application Publication
(10) Pub. No.: US 2026/0022124 A1
(43) Pub. Date: Jan. 22, 2026

(54) THERAPEUTIC COMPOUNDS AND METHODS

(71) Applicant: Regents of the University of Minnesota, Minneapolis, MN (US)

(72) Inventors: Ingrid Gunda Georg, Minneapolis, MN (US); Yutong Liu, Minneapolis, MN (US)

(21) Appl. No.: 19/271,417

(22) Filed: Jul. 16, 2025

Related U.S. Application Data
(60) Provisional application No. 63/672,087, filed on Jul. 16, 2024.

(51) Int. Cl. C07D 487/04; A61K 31/437; A61K 31/5025; A61K 31/55; A61P 15/16; C07D 471/04

(52) U.S. Cl. CPC C07D 487/04; A61K 31/437; A61K 31/5025; A61K 31/55; A61P 15/16; C07D 471/04

(57) ABSTRACT

The invention provides a compound of formula (I):

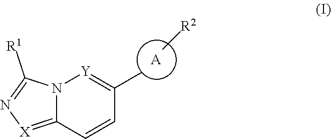

or a salt thereof, wherein R¹-R² and X and Y have any of the values described in the specification, as well as compositions comprising a compound of formula (I). The compounds are useful as contraceptive agents.

### CROSS-REFERENCE TO RELATED APPLICATION

This application claims priority to U.S. Provisional Application No. 63/672,087 that was filed on Jul. 16, 2024. The entire content of the application referenced above is hereby incorporated by reference herein.

### STATEMENT REGARDING FEDERALLY SPONSORED RESEARCH

This invention was made with government support under HD114221 awarded by the National Institutes of Health. The government has certain rights in the invention.

### BACKGROUND

Because of a global desire to reduce the size of families and the high rates of unintended pregnancies there is an urgent need for novel contraceptive methods (Bongaarts, J., Int. Perspect. Sex Reprod. Health 2011, 37, 209-216; and Bearak, J., et al., Lancet Glob. Health 2020, 8, e1152-e1161). While multiple female contraceptives are available, there are few options for men, although most men are willing to share the responsibility of contraception (Long, J. E., et al., J. Clin. Endocrinol. Metab. 2021, 106, e2381-e2392). During the past fifty years, significant efforts have been undertaken to develop hormonal male contraceptives, but side effects such as acne, low libido, weight change, and decreased HDL levels have hindered commercialization (Quill, T. A, et al., Proc. Natl. Acad. Sci. U.S.A. 2001, 98, 12527-12531).

Non hormonal contraceptives appear as an appealing alternative. A desired approach to achieve male contraception is targeting proteins that are essential for sperm function, specifically, those only expressed in sperm.

Currently there is a need for non-hormonal contraceptive agents.

### SUMMARY

An effective, non-steroid hormone-based reversible male contraceptive that exhibits few if any side effects, health risks, or further complications has been identified.

Accordingly, in one aspect a compound of formula (I):

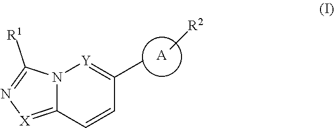

or a salt thereof, wherein:
   R¹ is a 6-10 membered aryl, (C₃-C₆)cycloalkyl, or a 5-10 membered heteroaryl, which 6-10 membered aryl, (C₃-C₆)cycloalkyl, and 5-10 membered heteroaryl is optionally substituted with one or more (e.g., 1, 2, 3, 4, or 5) groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more (e.g., 1, 2, 3, 4, or 5) groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
   X is N or CH;
   Y is N or CH;
   ring A is phenyl, 3-8 membered heterocyclic ring (e.g., 3, 4, 5, 6, 7, or 8 membered heterocyclic ring), or a 5-10 membered heteroaryl, which phenyl, 3-8 membered heterocyclic ring and 5-10 membered heteroaryl is optionally substituted with one or more (e.g., 1, 2, 3, 4, or 5) groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more (e.g., 1, 2, 3, 4, or 5) groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
   R² is —C(═O)—NR^{a}R^{b} or a 5-membered heteroaryl ring that is optionally substituted with one or more (e.g., 1, 2, 3, 4, or 5) groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more (e.g., 1, 2, 3, 4, or 5) groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy;
   R^{a} is H or (C₁-C₆)alkyl; and R^{b} is H, (C₁-C₆)alkyl, (C₃-C₆)cycloalkyl, or (C₃-C₆)cycloalkyl(C₁-C₆)alkyl, wherein any (C₁-C₆)alkyl, (C₃-C₆)cycloalkyl, and (C₃-C₆)cycloalkyl(C₁-C₆)alkyl is optionally substituted with one or more (e.g., 1, 2, 3, 4, or 5) groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, aryl, 5-10 membered heteroaryl, NR^{e}R^{f}, and (C₂-C₆)alkanoyloxy; or
   R^{a} and R^{b} together with the nitrogen to which they are attached form a 3-8 membered heterocyclic ring that is optionally substituted with one or more (e.g., 1, 2, 3, 4, or 5) groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy;
   each R^{c} and R^{d} is independently selected from the group consisting of H, (C₁-C₆)alkyl, (C₃-C₆)cycloalkyl, and (C₃-C₆)cycloalkyl(C₁-C₆)alkyl, or R^{c} and R^{d} together with the nitrogen to which they are attached form a 3-8 membered heterocyclic ring that is optionally substituted with one or more (e.g., 1, 2, 3, 4, or 5) groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy; and
   each R^{e} and R^{f} is independently selected from the group consisting of H, (C₁-C₆)alkyl, (C₃-C₆)cycloalkyl, and (C₃-C₆)cycloalkyl(C₁-C₆)alkyl, wherein (C₁-C₆)alkyl, (C₃-C₆)cycloalkyl, and (C₃-C₆)cycloalkyl(C₁-C₆)alkyl are optionally substituted with one or more (e.g., 1, 2, 3, 4, or 5) halo, or R^{e} and R^{f} together with the nitrogen to which they are attached form a 3-8 membered heterocyclic ring that is optionally substituted with one or more (e.g., 1, 2, 3, 4, or 5) groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is provided.

In one aspect a compound of formula (I):

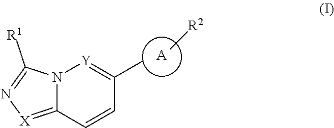

or a salt thereof, wherein:
   R¹ is a 6-10 membered aryl, (C₃-C₆)cycloalkyl, or a 5-10 membered heteroaryl, which 6-10 membered aryl, (C₃-C₆)cycloalkyl, and 5-10 membered heteroaryl is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
   X is N or CH;
   Y is N or CH;
   ring A is phenyl or a 3-8 membered heterocyclic ring, which phenyl and 3-8 membered heterocyclic ring is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
   R² is —C(═O)—NR^{a}R^{b} or a 5-membered heteroaryl ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy;
   R^{a} is H or (C₁-C₆)alkyl; and R^{b} is H, (C₁-C₆)alkyl, (C₃-C₆)cycloalkyl, or (C₃-C₆)cycloalkyl(C₁-C₆)alkyl, wherein any (C₁-C₆)alkyl, (C₃-C₆)cycloalkyl, and (C₃-C₆)cycloalkyl(C₁-C₆)alkyl is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, aryl, NR^{e}R^{f}, and (C₂-C₆)alkanoyloxy; or
   R^{a} and R^{b} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy;
   each R^{c} and R^{d} is independently selected from the group consisting of H, (C₁-C₆)alkyl, (C₃-C₆)cycloalkyl, and (C₃-C₆)cycloalkyl(C₁-C₆)alkyl, or R^{c} and R^{d} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy; and
   each R^{e} and R^{f} is independently selected from the group consisting of H, (C₁-C₆)alkyl, (C₃-C₆)cycloalkyl, and (C₃-C₆)cycloalkyl(C₁-C₆)alkyl, or R^{e} and R^{f} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is also provided.

A pharmaceutical composition comprising a compound of formula (I) or a pharmaceutically acceptable salt thereof, and a pharmaceutically acceptable excipient is also provided.

A method to reduce the likelihood of conception following intercourse between a male subject and a female subject, comprising administering a compound of formula (I) or pharmaceutically acceptable salt thereof to the male subject or to the female subject is also provided.

A method to reduce sperm motility in a male subject, comprising administering a compound of formula (I) or pharmaceutically acceptable salt thereof to the male subject is also provided.

A method to reduce sperm motility following intercourse between a male subject and a female subject, comprising administering a compound of formula (I) or pharmaceutically acceptable salt thereof to the female subject is also provided.

A method to produce reversible infertility in a male subject, comprising administering a compound of formula (I) or pharmaceutically acceptable salt thereof to the male subject is also provided.

A compound of formula (I) or pharmaceutically acceptable salt thereof for use in medical therapy.

A compound of formula (I) or pharmaceutically acceptable salt thereof to reduce the likelihood of conception following intercourse between a male subject and a female subject is also provided.

A compound of formula (I) or pharmaceutically acceptable salt thereof to reduce sperm motility in a male subject is also provided.

A compound of formula (I) or pharmaceutically acceptable salt thereof to reduce sperm motility following intercourse between a male subject and a female subject is also provided.

A compound of formula (I) or pharmaceutically acceptable salt thereof to produce reversible infertility in a male subject is also provided.

The use of a compound of formula (I) or pharmaceutically acceptable salt thereof to prepare a medicament to reduce the likelihood of conception following intercourse between a male subject and a female subject is also provided.

The use of a compound of formula (I) or pharmaceutically acceptable salt thereof to prepare a medicament to reduce sperm motility in a male subject is also provided.

The use of a compound of formula (I) or pharmaceutically acceptable salt thereof to prepare a medicament to reduce sperm motility following intercourse between a male subject and a female subject is also provided.

The use of a compound of formula (I) or pharmaceutically acceptable salt thereof to prepare a medicament to produce reversible infertility in a male subject is also provided.

A kit comprising packaging material that contains a compound or pharmaceutically acceptable salt as described in any one of claims 1-32 and instructions for use of the compound or pharmaceutically acceptable salt as a contraceptive (e.g., to reduce the likelihood of conception following intercourse, to reduce sperm motility in a male subject, to reduce sperm motility following intercourse between a male subject and a female subject, or to produce reversible infertility in a male subject) is also provided.

Processes and intermediates disclosed herein that are useful for preparing a compound of formula (I) or a salt thereof are also provided.

### DETAILED DESCRIPTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, (C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to alkyl groups attached to the remainder of the molecule via an oxygen atom (“oxy”).

The term “alkylthio” refers to alkyl groups attached to the remainder of the molecule via a thio group.

The term “cycloalkyl” refers to a saturated or partially unsaturated (non-aromatic) all carbon ring having 3 to 8 carbon atoms (i.e., (C₃-C₈) carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur(S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl) ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” includes mammals, fish, amphibians, reptiles, birds and invertebrates. The term “mammal” includes humans, higher non-human primates, rodents, domestic, cows, horses, pigs, sheep, dogs and cats. In one embodiment, the subject is an animal. In one embodiment, the subject is a mammal. In one embodiment, the subject is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for ring A is phenyl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for ring A is a 3-8 membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific compound or salt is a compound of formula (Ia):

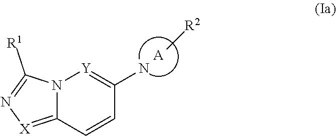

or a salt thereof:
   wherein ring A is a 3-8 membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for ring A is a 4-, 5-, or 6-membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for ring A is a 4-membered heterocyclic ring.

A specific value for ring A is a 5-membered heterocyclic ring.

A specific value for ring A is:

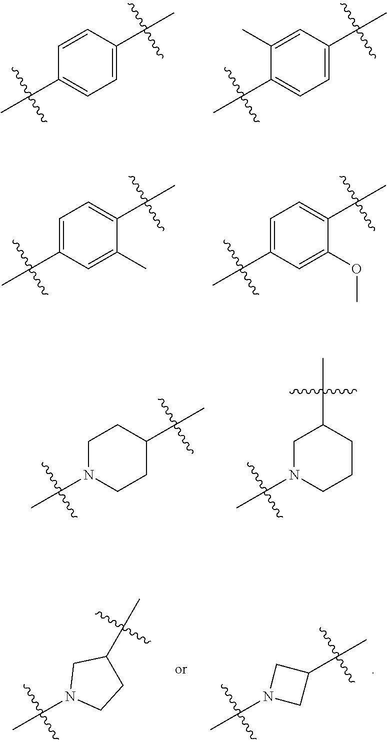

A specific value for R¹ is a 6-10 membered aryl, that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R¹ is a (C₃-C₆)cycloalkyl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R¹ is a 5-10 membered heteroaryl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R¹ is a phenyl or naphthyl, which phenyl and naphthyl is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R¹ is cyclohexyl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R¹ is a phenyl or naphthyl, which phenyl and naphthyl is optionally substituted with one or more groups independently selected from the group consisting of halo, (C₁-C₆)alkyl, and (C₁-C₆)alkoxy, wherein any (C₁-C₆)alkyl and (C₁-C₆)alkoxy is optionally substituted with one or more groups independently selected from the group consisting of halo.

A specific value for R¹ is cyclohexyl that is optionally substituted with one or more groups independently selected from the group consisting of halo, (C₁-C₆)alkyl, and (C₁-C₆)alkoxy, wherein any (C₁-C₆)alkyl and (C₁-C₆)alkoxy is optionally substituted with one or more groups independently selected from the group consisting of halo.

A specific value for R¹ is 4-methylphenyl, 3-methylphenyl, 2-methylphenyl, 4-fluorophenyl, 4-methoxyphenyl, 4-trifluoromethylphenyl, 4-chlorophenyl, 3,4-dichlorophenyl, cyclohexyl, or naphthyl.

A specific value for X is N.

A specific value for X is CH.

A specific value for Y is N.

A specific value for Y is CH.

In one embodiment, X is N and Y is N.

A specific value for R² is —NR^{a}R^{b}.

A specific value for R^{a} is H or (C₁-C₆)alkyl; and R^{b} is (C₁-C₆)alkyl, (C₃-C₆)cycloalkyl, or (C₃-C₆)cycloalkyl(C₁-C₆)alkyl, wherein any (C₁-C₆)alkyl, (C₃-C₆)cycloalkyl, and (C₃-C₆)cycloalkyl(C₁-C₆)alkyl is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{e}R^{f}, and (C₂-C₆)alkanoyloxy.

A specific value for R^{a} is H or (C₁-C₆)alkyl; and R^{b} is (C₁-C₆)alkyl that is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{e}R^{f}, and (C₂-C₆)alkanoyloxy.

A specific value for R^{a} is H or (C₁-C₆)alkyl; and R^{b} is (C₁-C₆)alkyl that is substituted with NR^{e}R^{f}.

A specific value for R^{a} is H.

A specific value for R^{a} and R^{b} together with the nitrogen to which they are attached is a 5-8 membered ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for R^{a} and R^{b} together with the nitrogen to which they are attached is a 5-8 membered ring.

A specific value for R² is a 5-membered heteroaryl ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy.

A specific value for R² is a 5-membered heteroaryl ring that is optionally substituted with one or more groups independently selected from the group consisting of halo, and (C₁-C₆)alkyl that is optionally substituted with one or more groups independently selected from the group consisting of halo, hydroxy, carboxy, (C₁-C₆)alkoxy, and NR^{c}R^{d}.

A specific value for R² is:

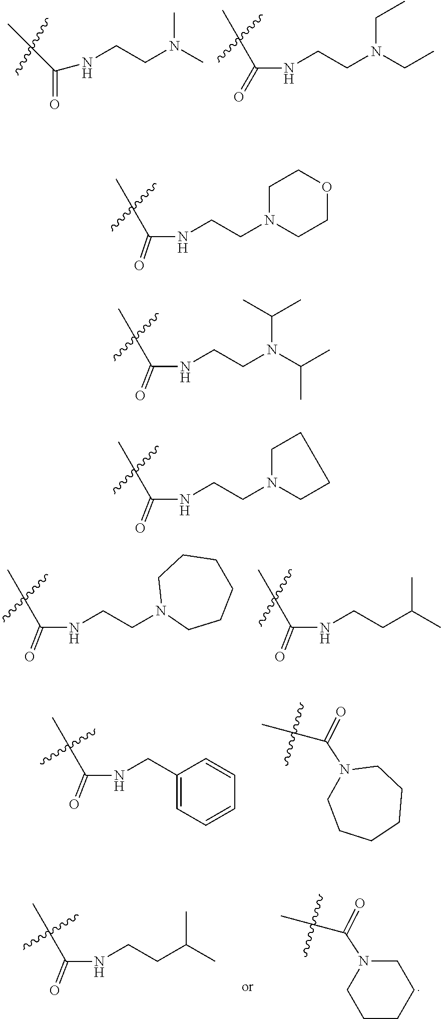

A specific value for R² is:

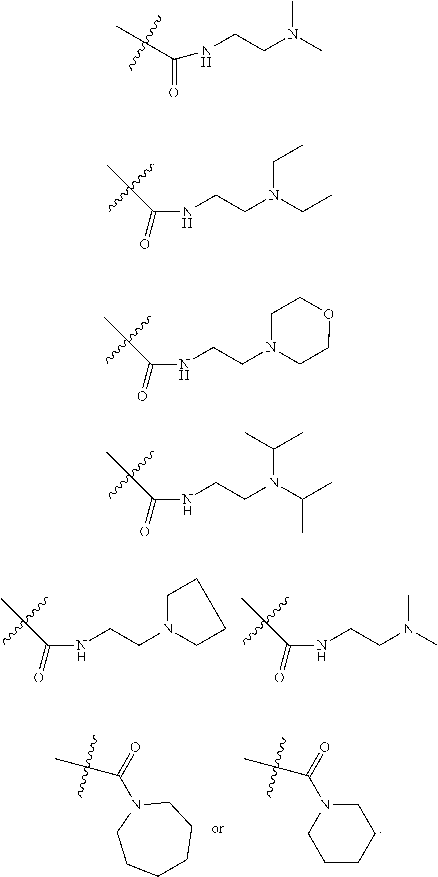

One embodiment provides a compound of formula (Ia):

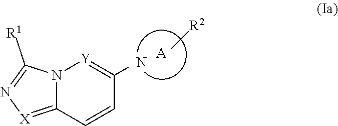

or a salt thereof:
   wherein ring A is a 4-, 5-, or 6-membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

A specific value for ring A is:

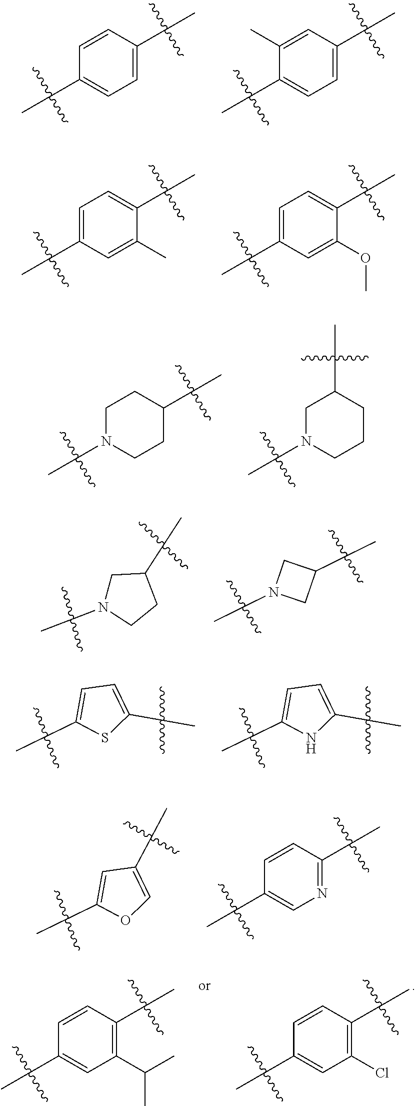

A specific value for R¹ is 4-methylphenyl, 3-methylphenyl, 2-methylphenyl, 4-fluorophenyl, 4-methoxyphenyl, 4-trifluoromethylphenyl, 4-chlorophenyl, 3,4-dichlorophenyl, cyclohexyl, naphthyl, 3-trifluoromethylphenyl, 3-methoxyphenyl, or 3-chlorophenyl.

In one embodiment X is N and Y is N or X is CH and Y is CH.

A specific value for R² is:

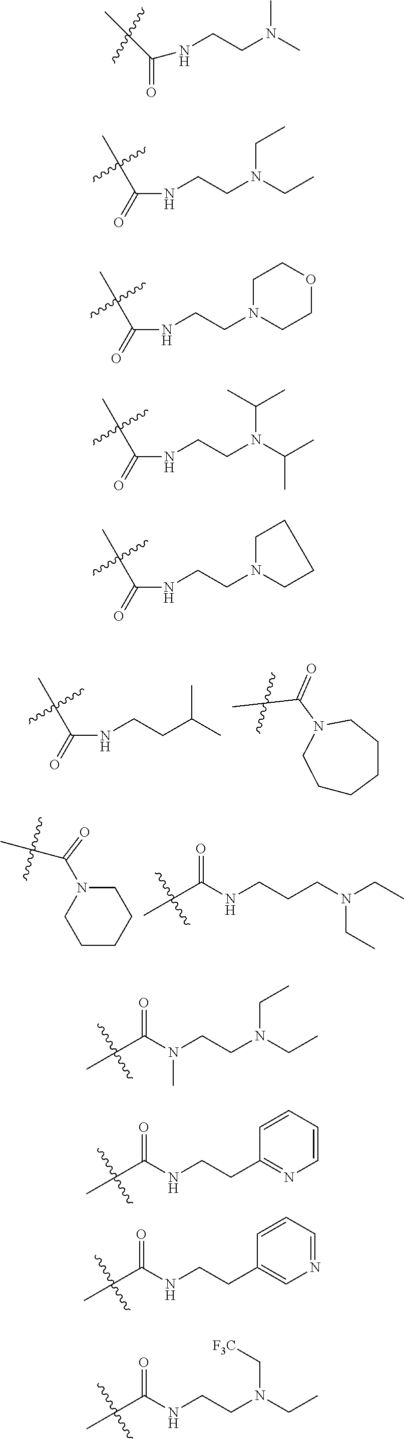

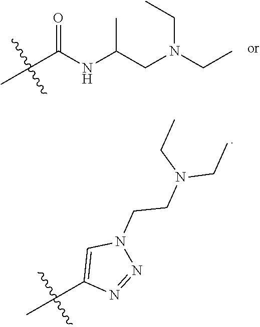 [SMILES: *C(=O)NC(C)CN(CC)CC ; *c1cn(CCN(CC)CC)nn1]

One embodiment provides a compound or salt of selected from the group consisting of:

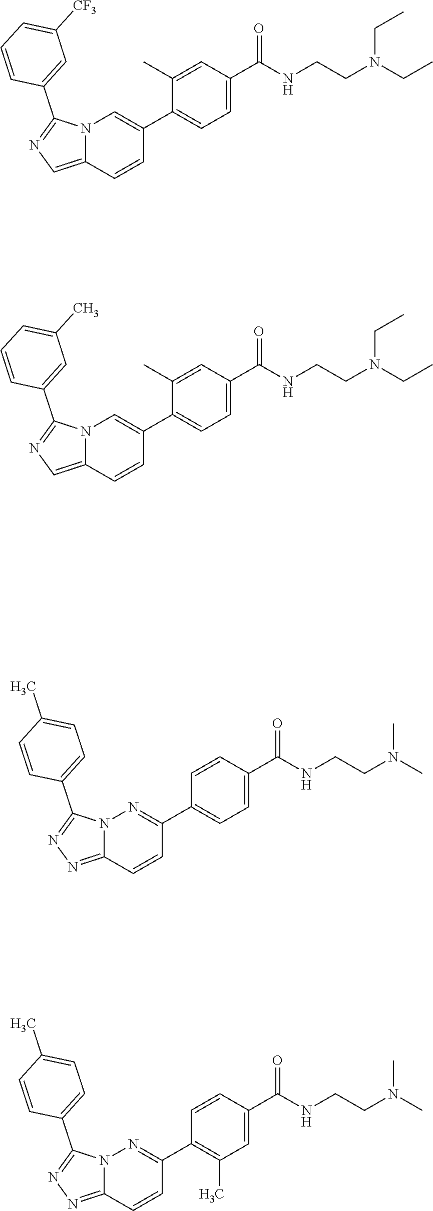 [SMILES: CCN(CC)CCNC(=O)c1ccc(-c2ccc3cnc(-c4cccc(C(F)(F)F)c4)n3c2)c(C)c1 ; CCN(CC)CCNC(=O)c1ccc(-c2ccc3cnc(-c4cccc(C)c4)n3c2)c(C)c1 ; Cc1ccc(-c2nnc3ccc(-c4ccc(C(=O)NCCN(C)C)cc4)nn23)cc1 ; Cc1ccc(-c2nnc3ccc(-c4ccc(C(=O)NCCN(C)C)cc4C)nn23)cc1]

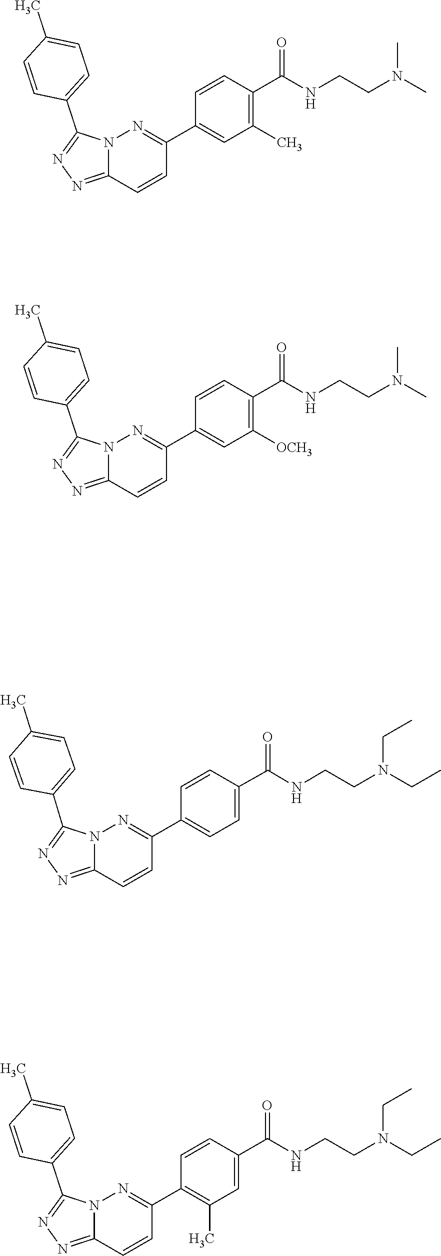 [SMILES: Cc1ccc(-c2nnc3ccc(-c4ccc(C(=O)NCCN(C)C)c(C)c4)nn23)cc1 ; COc1cc(-c2ccc3nnc(-c4ccc(C)cc4)n3n2)ccc1C(=O)NCCN(C)C ; CCN(CC)CCNC(=O)c1ccc(-c2ccc3nnc(-c4ccc(C)cc4)n3n2)cc1 ; CCN(CC)CCNC(=O)c1ccc(-c2ccc3nnc(-c4ccc(C)cc4)n3n2)c(C)c1]

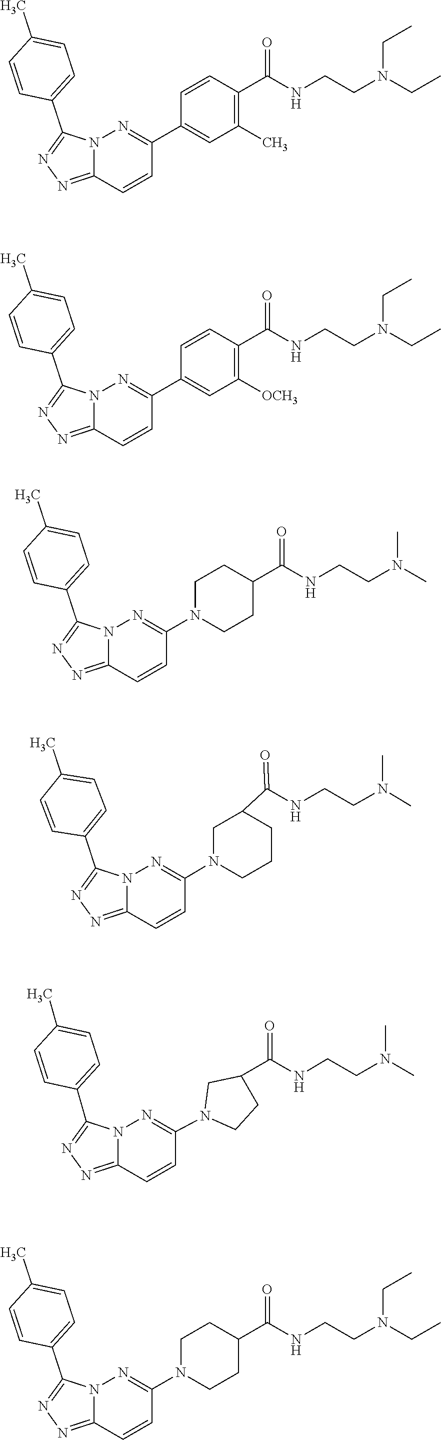

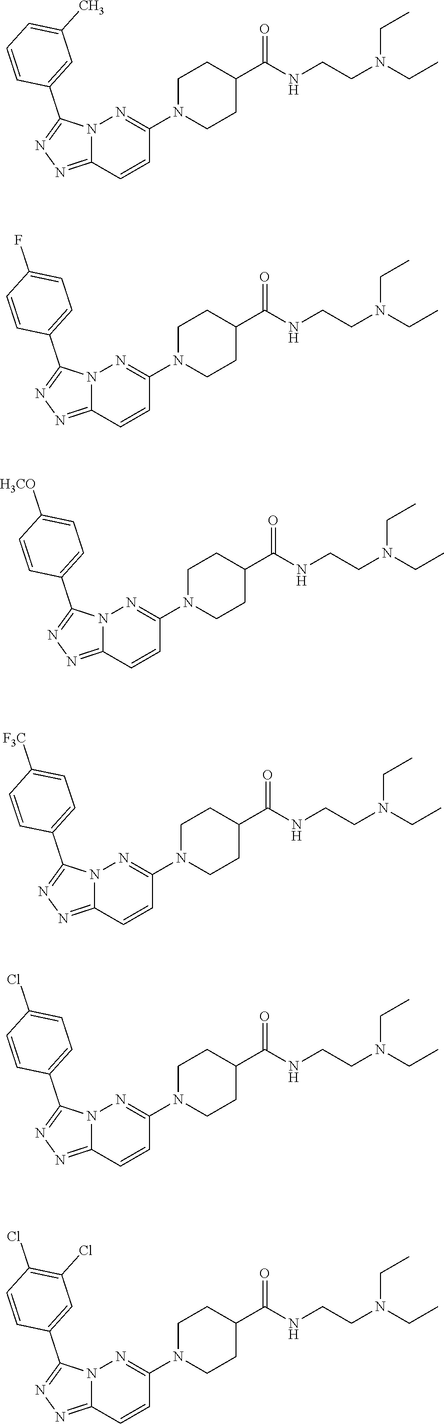 [SMILES: CCN(CC)CCNC(=O)C1CCN(c2ccc3nnc(-c4cccc(C)c4)n3n2)CC1 ; CCN(CC)CCNC(=O)C1CCN(c2ccc3nnc(-c4ccc(F)cc4)n3n2)CC1 ; CCN(CC)CCNC(=O)C1CCN(c2ccc3nnc(-c4ccc(OC)cc4)n3n2)CC1 ; CCN(CC)CCNC(=O)C1CCN(c2ccc3nnc(-c4ccc(C(F)(F)F)cc4)n3n2)CC1 ; CCN(CC)CCNC(=O)C1CCN(c2ccc3nnc(-c4ccc(Cl)cc4)n3n2)CC1 ; CCN(CC)CCNC(=O)C1CCN(c2ccc3nnc(-c4ccc(Cl)c(Cl)c4)n3n2)CC1]

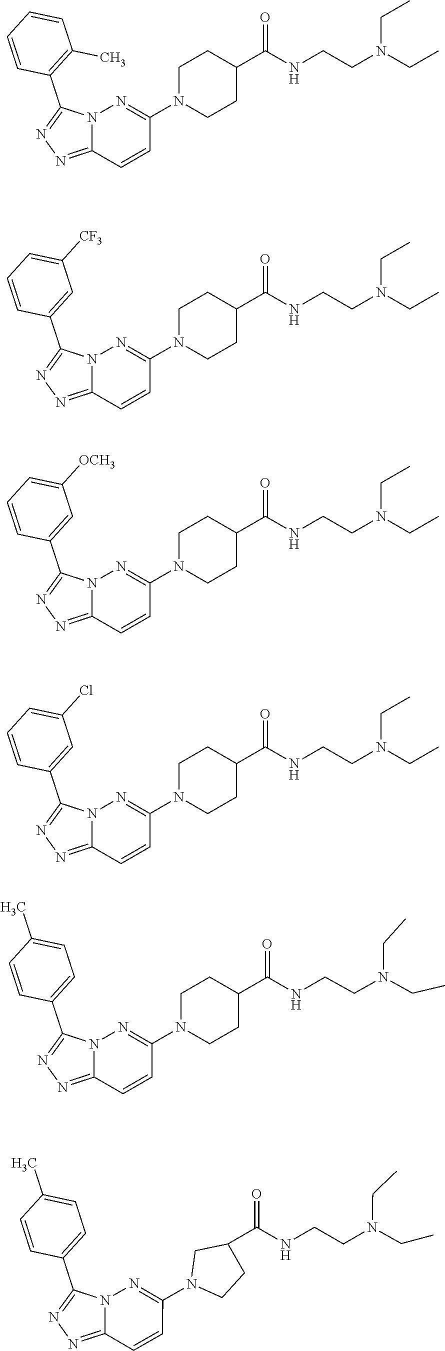 [SMILES: CCN(CC)CCNC(=O)C1CCN(c2ccc3nnc(-c4ccccc4C)n3n2)CC1 ; CCN(CC)CCNC(=O)C1CCN(c2ccc3nnc(-c4cccc(C(F)(F)F)c4)n3n2)CC1 ; CCN(CC)CCNC(=O)C1CCN(c2ccc3nnc(-c4cccc(OC)c4)n3n2)CC1 ; CCN(CC)CCNC(=O)C1CCN(c2ccc3nnc(-c4cccc(Cl)c4)n3n2)CC1 ; CCN(CC)CCNC(=O)C1CCN(c2ccc3nnc(-c4ccc(C)cc4)n3n2)CC1 ; CCN(CC)CCNC(=O)C1CCN(c2ccc3nnc(-c4ccc(C)cc4)n3n2)C1]

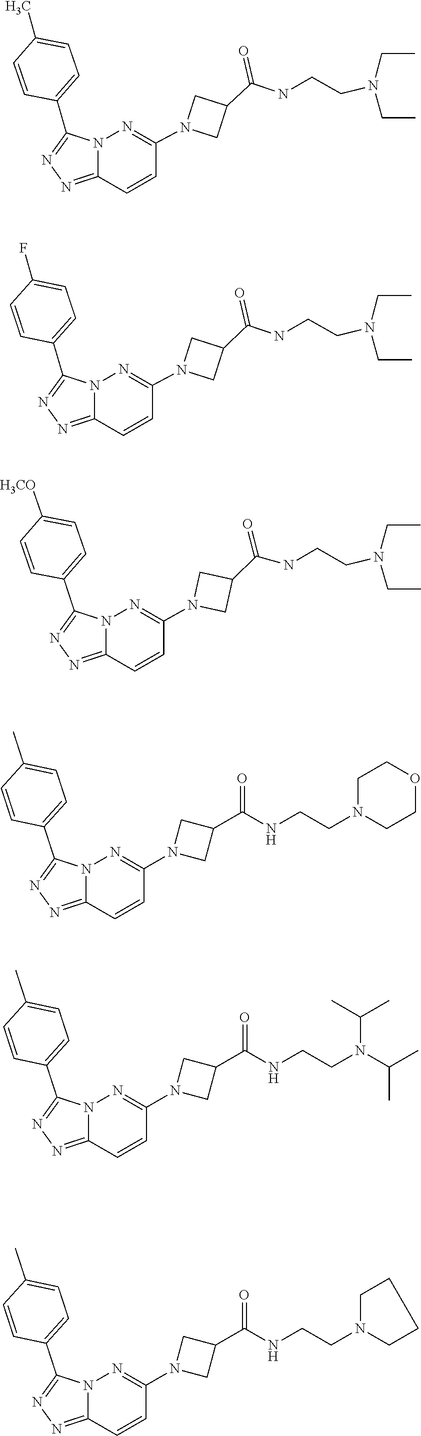

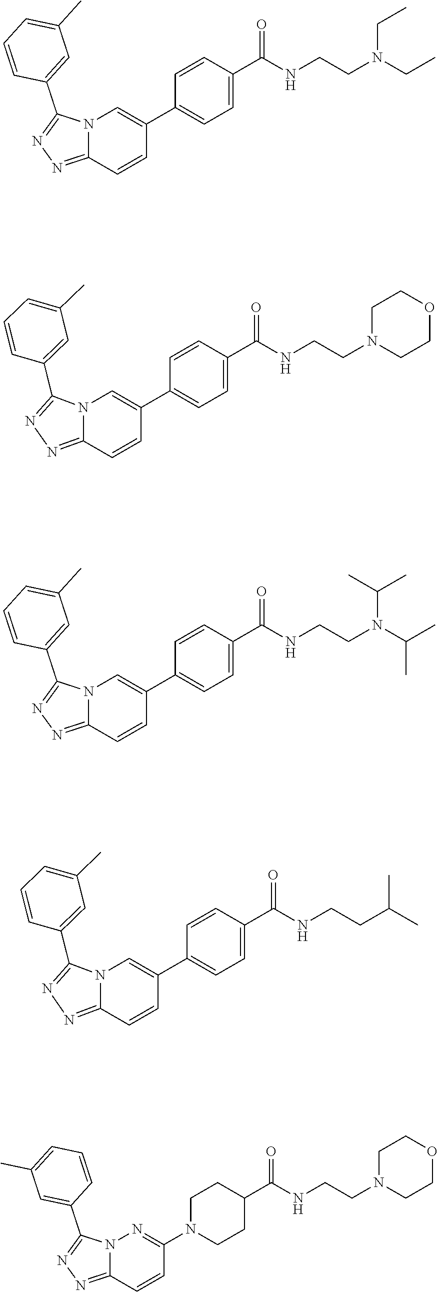

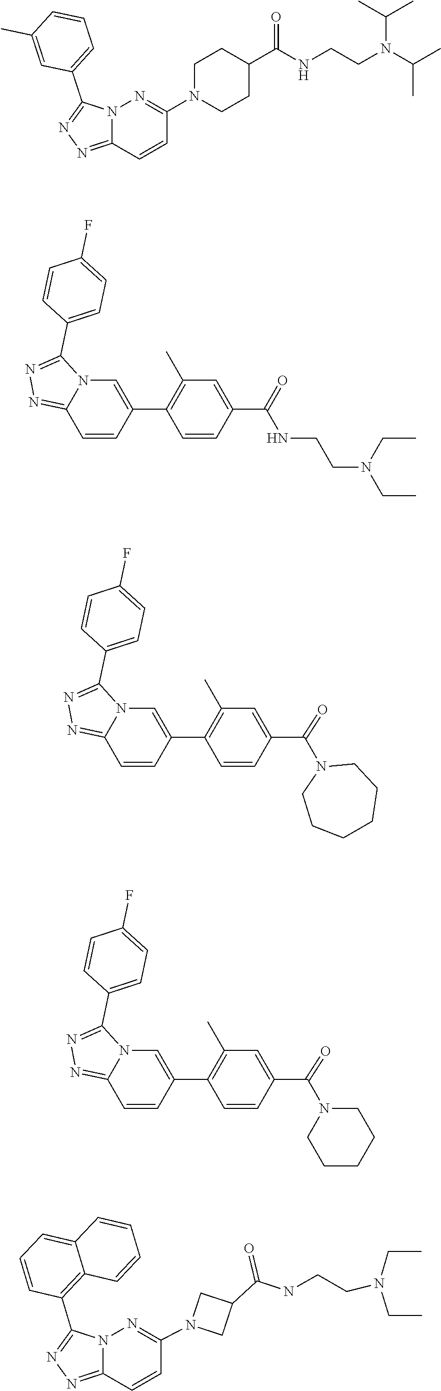

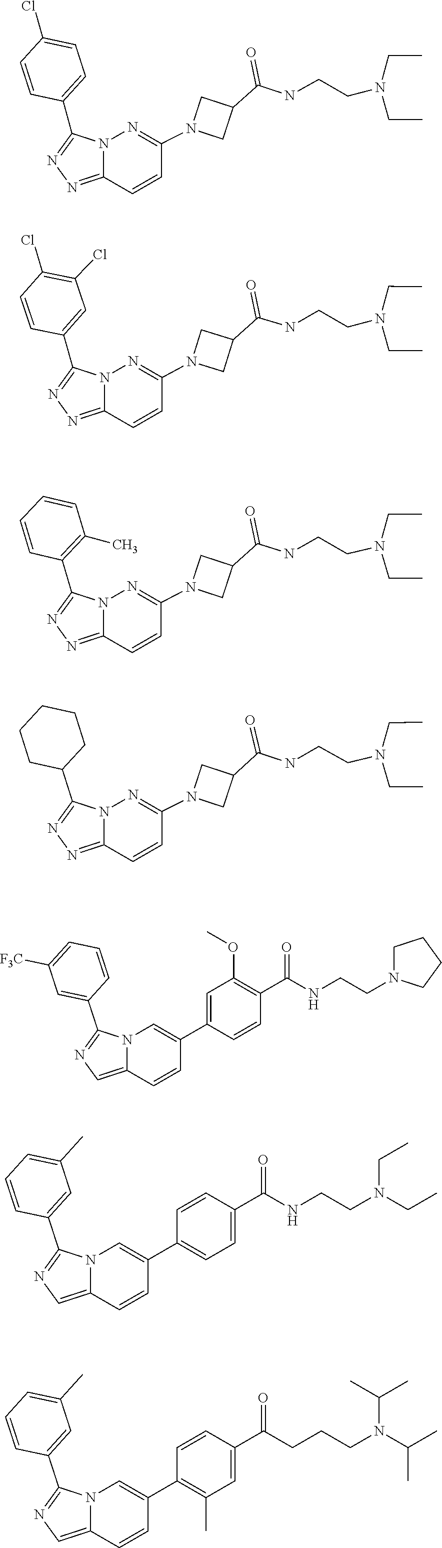

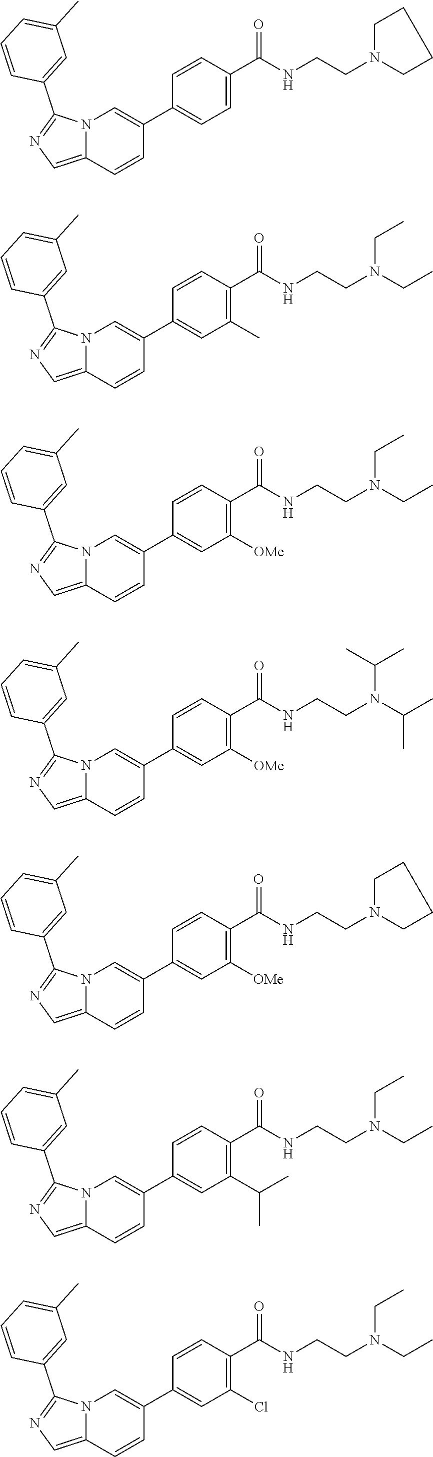

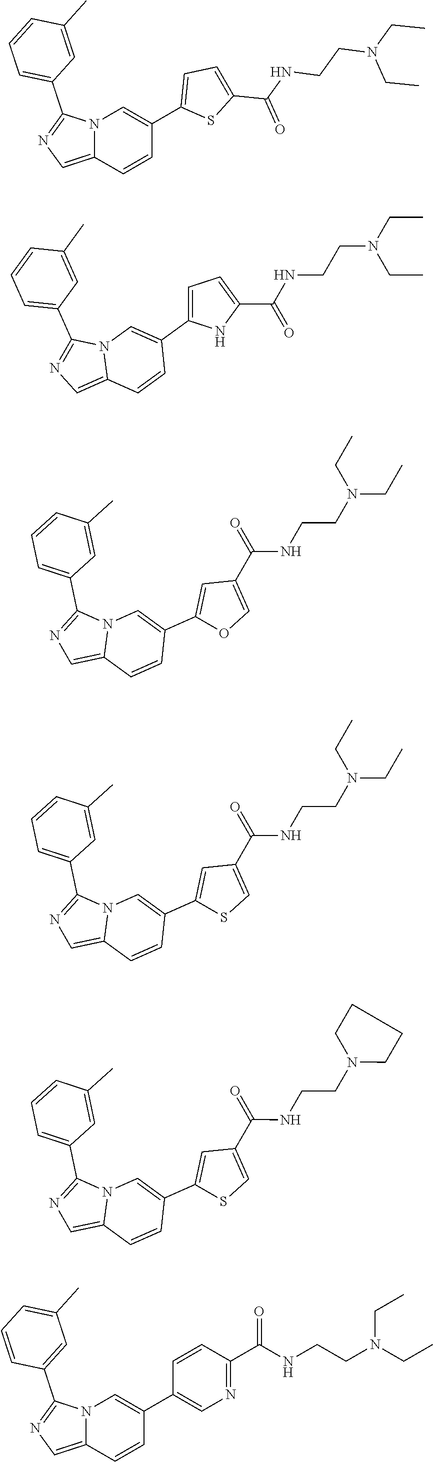 [SMILES: CCN(CC)CCNC(=O)c1ccc(-c2ccc3cnc(-c4cccc(C)c4)n3c2)s1 ; CCN(CC)CCNC(=O)c1ccc(-c2ccc3cnc(-c4cccc(C)c4)n3c2)[nH]1 ; CCN(CC)CCNC(=O)c1coc(-c2ccc3cnc(-c4cccc(C)c4)n3c2)c1 ; CCN(CC)CCNC(=O)c1csc(-c2ccc3cnc(-c4cccc(C)c4)n3c2)c1 ; Cc1cccc(-c2ncc3ccc(-c4cc(C(=O)NCCN5CCCC5)cs4)cn23)c1 ; CCN(CC)CCNC(=O)c1ccc(-c2ccc3cnc(-c4cccc(C)c4)n3c2)cn1]

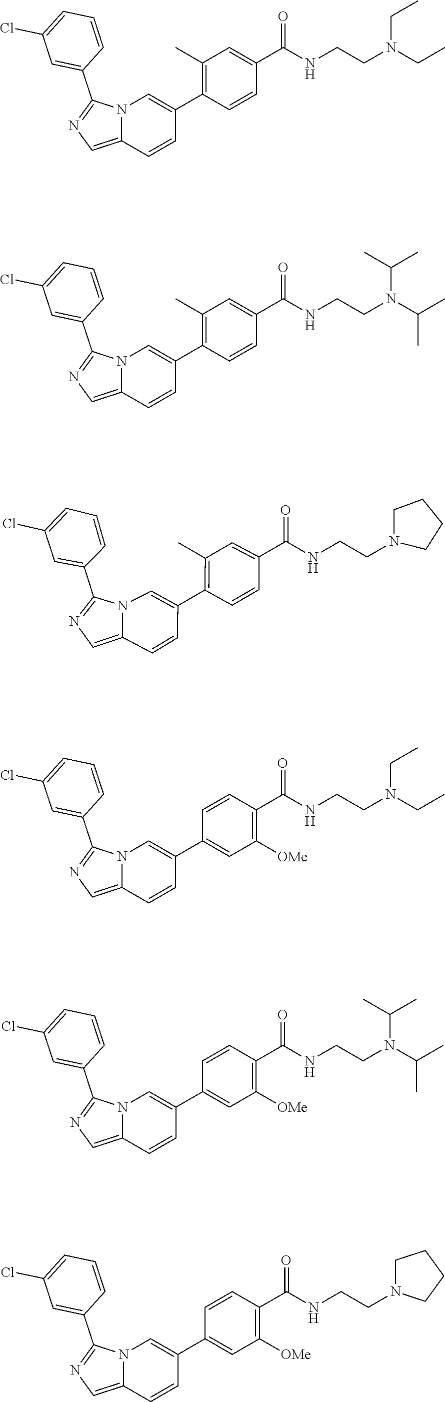 [SMILES: CCN(CC)CCNC(=O)c1ccc(-c2ccc3cnc(-c4cccc(Cl)c4)n3c2)c(C)c1 ; Cc1cc(C(=O)NCCN(C(C)C)C(C)C)ccc1-c1ccc2cnc(-c3cccc(Cl)c3)n2c1 ; Cc1cc(C(=O)NCCN2CCCC2)ccc1-c1ccc2cnc(-c3cccc(Cl)c3)n2c1 ; CCN(CC)CCNC(=O)c1ccc(-c2ccc3cnc(-c4cccc(Cl)c4)n3c2)cc1OC ; COc1cc(-c2ccc3cnc(-c4cccc(Cl)c4)n3c2)ccc1C(=O)NCCN(C(C)C)C(C)C ; COc1cc(-c2ccc3cnc(-c4cccc(Cl)c4)n3c2)ccc1C(=O)NCCN1CCCC1]

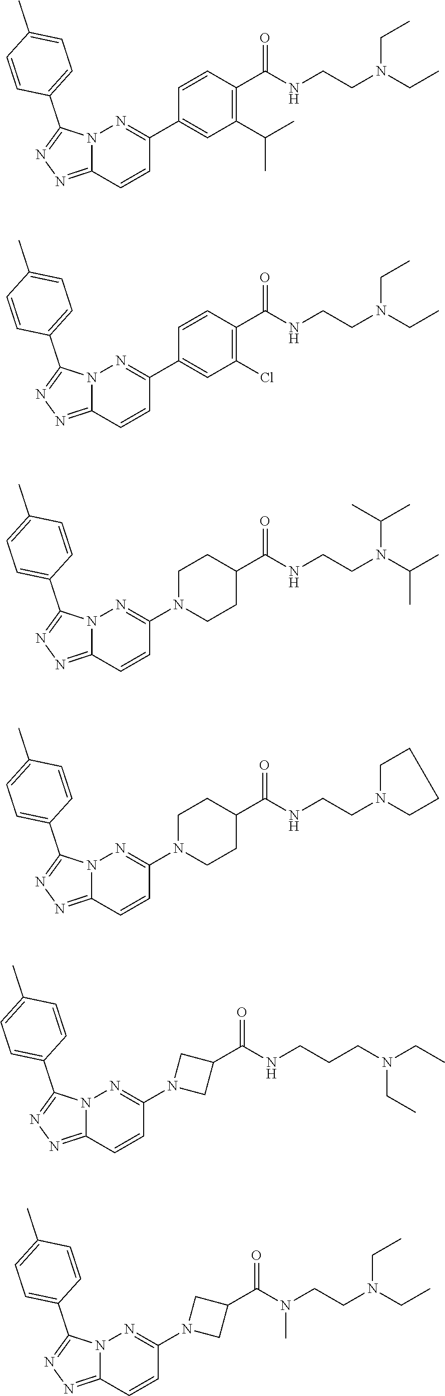 [SMILES: CCN(CC)CCNC(=O)c1ccc(-c2ccc3nnc(-c4ccc(C)cc4)n3n2)cc1C(C)C ; CCN(CC)CCNC(=O)c1ccc(-c2ccc3nnc(-c4ccc(C)cc4)n3n2)cc1Cl ; Cc1ccc(-c2nnc3ccc(N4CCC(C(=O)NCCN(C(C)C)C(C)C)CC4)nn23)cc1 ; Cc1ccc(-c2nnc3ccc(N4CCC(C(=O)NCCN5CCCC5)CC4)nn23)cc1 ; CCN(CC)CCCNC(=O)C1CN(c2ccc3nnc(-c4ccc(C)cc4)n3n2)C1 ; CCN(CC)CCN(C)C(=O)C1CN(c2ccc3nnc(-c4ccc(C)cc4)n3n2)C1]

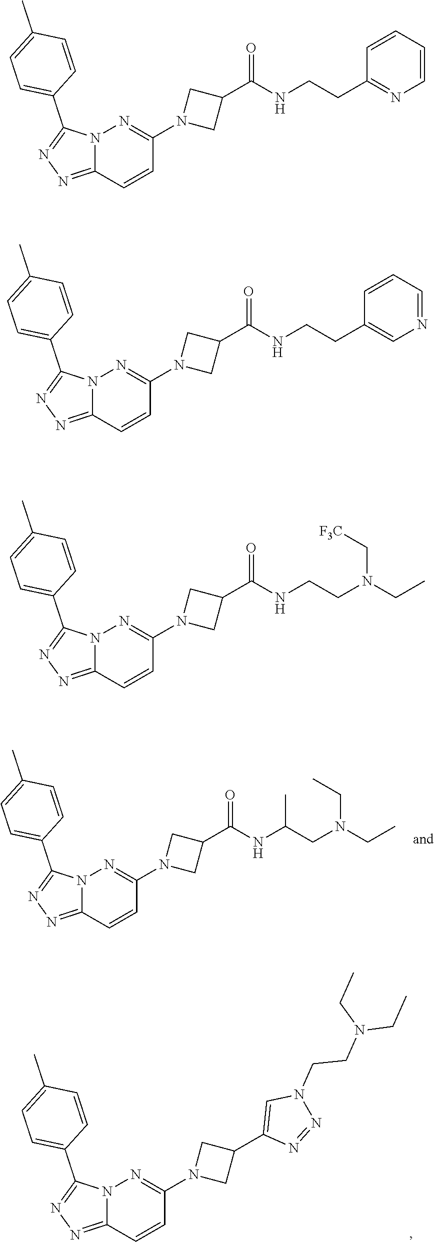 [SMILES: Cc1ccc(-c2nnc3ccc(N4CC(C(=O)NCCc5ccccn5)C4)nn23)cc1 ; Cc1ccc(-c2nnc3ccc(N4CC(C(=O)NCCc5cccnc5)C4)nn23)cc1 ; CCN(CCNC(=O)C1CN(c2ccc3nnc(-c4ccc(C)cc4)n3n2)C1)CC(F)(F)F ; CCN(CC)CC(C)NC(=O)C1CN(c2ccc3nnc(-c4ccc(C)cc4)n3n2)C1 ; CCN(CC)CCn1cc(C2CN(c3ccc4nnc(-c5ccc(C)cc5)n4n3)C2)nn1]

and salts thereof.

In one embodiment, a method to reduce the likelihood of conception following intercourse between a male subject and a female subject, comprising administering a compound of formula (I) or pharmaceutically acceptable salt thereof to the male subject or to the female subject is provided. In one embodiment, the compound or the pharmaceutically acceptable salt is administered to the female subject. In one embodiment, the compound or pharmaceutically acceptable is administered to the female subject prior to intercourse. In one embodiment, the compound or pharmaceutically acceptable salt is administered to the female subject orally. In one embodiment, the compound or pharmaceutically acceptable salt is administered to the female subject topically. In one embodiment, the compound or pharmaceutically acceptable salt is administered to the female subject intravaginally. In one embodiment, the compound or the pharmaceutically acceptable salt is administered to the male subject. In one embodiment, the compound or pharmaceutically acceptable is administered to the male subject prior to intercourse. In one embodiment, the compound or pharmaceutically acceptable salt is administered to the male subject orally. In one embodiment, the compound or pharmaceutically acceptable salt is administered to the male subject topically.

In one embodiment, a method to reduce sperm motility in a male subject, comprising administering a compound of formula (I) or pharmaceutically acceptable salt thereof to the male subject is provided. In one embodiment, the compound or pharmaceutically acceptable is administered to the male subject prior to intercourse. In one embodiment, the compound or pharmaceutically acceptable salt is administered to the male subject orally. In one embodiment, the compound or pharmaceutically acceptable salt is administered to the male subject topically.

In one embodiment, a method to reduce sperm motility following intercourse between a male subject and a female subject, comprising administering a compound of formula (I) or pharmaceutically acceptable salt thereof to the female subject is provided. In one embodiment, the compound or pharmaceutically acceptable is administered to the female subject prior to intercourse. In one embodiment, the compound or pharmaceutically acceptable salt is administered to the female subject orally. In one embodiment, the compound or pharmaceutically acceptable salt is administered to the female subject topically. In one embodiment, the compound or pharmaceutically acceptable salt is administered intravaginally.

In one embodiment, a method to produce reversible infertility in a male subject, comprising administering a compound of formula (I) or pharmaceutically acceptable salt thereof to the male subject is provided. In one embodiment, the compound or pharmaceutically acceptable salt is administered to the male subject orally. In one embodiment, the compound or pharmaceutically acceptable salt is administered to the male subject topically.

In one embodiment, a compound of formula (I) or pharmaceutically acceptable salt thereof to reduce the likelihood of conception following intercourse between a male subject and a female subject is provided. In one embodiment, the compound or the pharmaceutically acceptable salt is administered to the female subject. In one embodiment, the compound or pharmaceutically acceptable is administered to the female subject prior to intercourse. In one embodiment, the compound or pharmaceutically acceptable salt is administered to the female subject orally. In one embodiment, the compound or pharmaceutically acceptable salt is administered to the female subject topically. In one embodiment, the compound or pharmaceutically acceptable salt is administered to the female subject intravaginally. In one embodiment, the compound or pharmaceutically acceptable salt is administered to the male subject. In one embodiment, the compound or pharmaceutically acceptable is administered to the male subject prior to intercourse. In one embodiment, the compound or pharmaceutically acceptable salt is administered to the male subject orally. In one embodiment, the compound or pharmaceutically acceptable salt is administered to the male subject topically.

In one embodiment, a compound of formula (I) or pharmaceutically acceptable salt thereof to reduce sperm motility in a male subject is provided. In one embodiment, the compound or pharmaceutically acceptable is administered to the male subject prior to intercourse. In one embodiment, the compound or pharmaceutically acceptable salt is administered to the male subject orally. In one embodiment, the compound or pharmaceutically acceptable salt is administered to the male subject topically.

In one embodiment, a compound of formula (I) or pharmaceutically acceptable salt thereof to reduce sperm motility following intercourse between a male subject and a female subject is provided. In one embodiment, the compound or pharmaceutically acceptable is administered to the female subject. In one embodiment, the compound or pharmaceutically acceptable is administered to the female subject prior to intercourse. In one embodiment, the compound or pharmaceutically acceptable salt is administered to the female subject orally. In one embodiment, the compound or pharmaceutically acceptable salt is administered to the female subject topically. In one embodiment, the compound or pharmaceutically acceptable salt is administered intravaginally.

In one embodiment, a compound of formula (I) or pharmaceutically acceptable salt thereof to produce reversible infertility in a male subject is provided. In one embodiment, the compound or pharmaceutically acceptable salt is administered to the male subject orally. In one embodiment, the compound or pharmaceutically acceptable salt is administered to the male subject topically.

In one embodiment, a use of a compound of formula (I) or pharmaceutically acceptable salt thereof to prepare a medicament to reduce the likelihood of conception following intercourse between a male subject and a female subject is provided. In one embodiment, the medicament is formulated for administration to the female subject. In one embodiment, the medicament is formulated for administration to the female subject prior to intercourse. In one embodiment, the medicament is formulated for administration to the female subject orally. In one embodiment, the medicament is formulated for administration to the female subject topically. In one embodiment, the medicament is formulated for administration to the female subject intravaginally. In one embodiment, the medicament is formulated for administration to the male subject. In one embodiment, the medicament is formulated for administration to the male subject prior to intercourse. In one embodiment, the medicament is formulated for administration to the male subject orally. In one embodiment, the medicament is formulated for administration to the male subject topically.

In one embodiment, a use of a compound of formula (I) or pharmaceutically acceptable salt thereof to prepare a medicament to reduce sperm motility in a male subject is provided. In one embodiment, the medicament is formulated for administration to the male subject prior to intercourse. In one embodiment, the medicament is formulated for administration to the male subject orally. In one embodiment, the medicament is formulated for administration to the male subject topically.

In one embodiment, a use of a compound of formula (I) or pharmaceutically acceptable salt thereof to prepare a medicament to reduce sperm motility following intercourse between a male subject and a female subject is provided. In one embodiment, the medicament is formulated for administration to the female subject. In one embodiment, the medicament is formulated for administration to the female subject prior to intercourse. In one embodiment, the medicament is formulated for administration to the female subject orally. In one embodiment, the medicament is formulated for administration to the female subject topically. In one embodiment, the medicament is formulated for administration to the female subject intravaginally.

In one embodiment, a use of a compound of formula (I) or pharmaceutically acceptable salt thereof to prepare a medicament to produce reversible infertility in a male subject is provided. In one embodiment, the medicament is formulated for administration to the male subject orally. In one embodiment, the medicament is formulated for administration to the male subject topically.

Processes for preparing compounds of formula (I) are provided as further embodiments of the invention and are illustrated by the following procedures in which the meanings of the generic radicals are as given above unless otherwise qualified.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The invention will now be illustrated by the following non-limiting Examples.

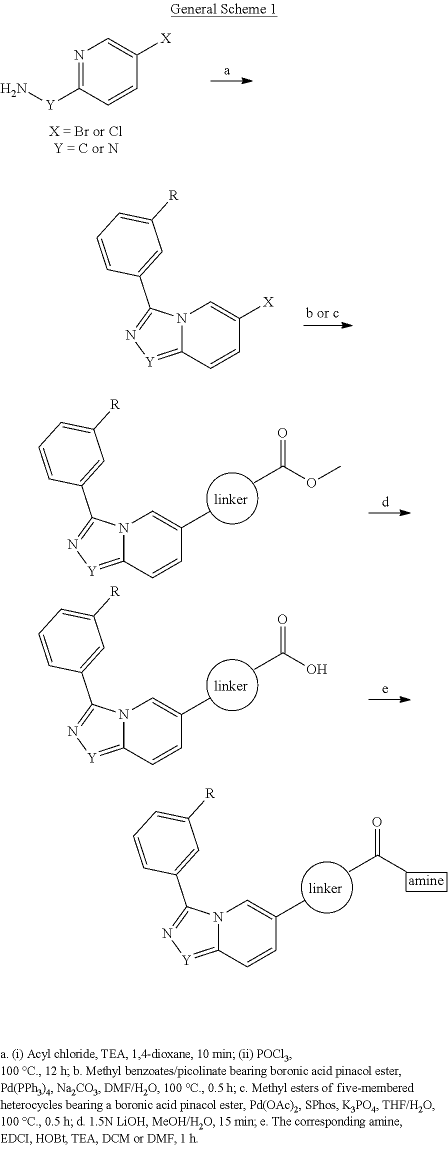

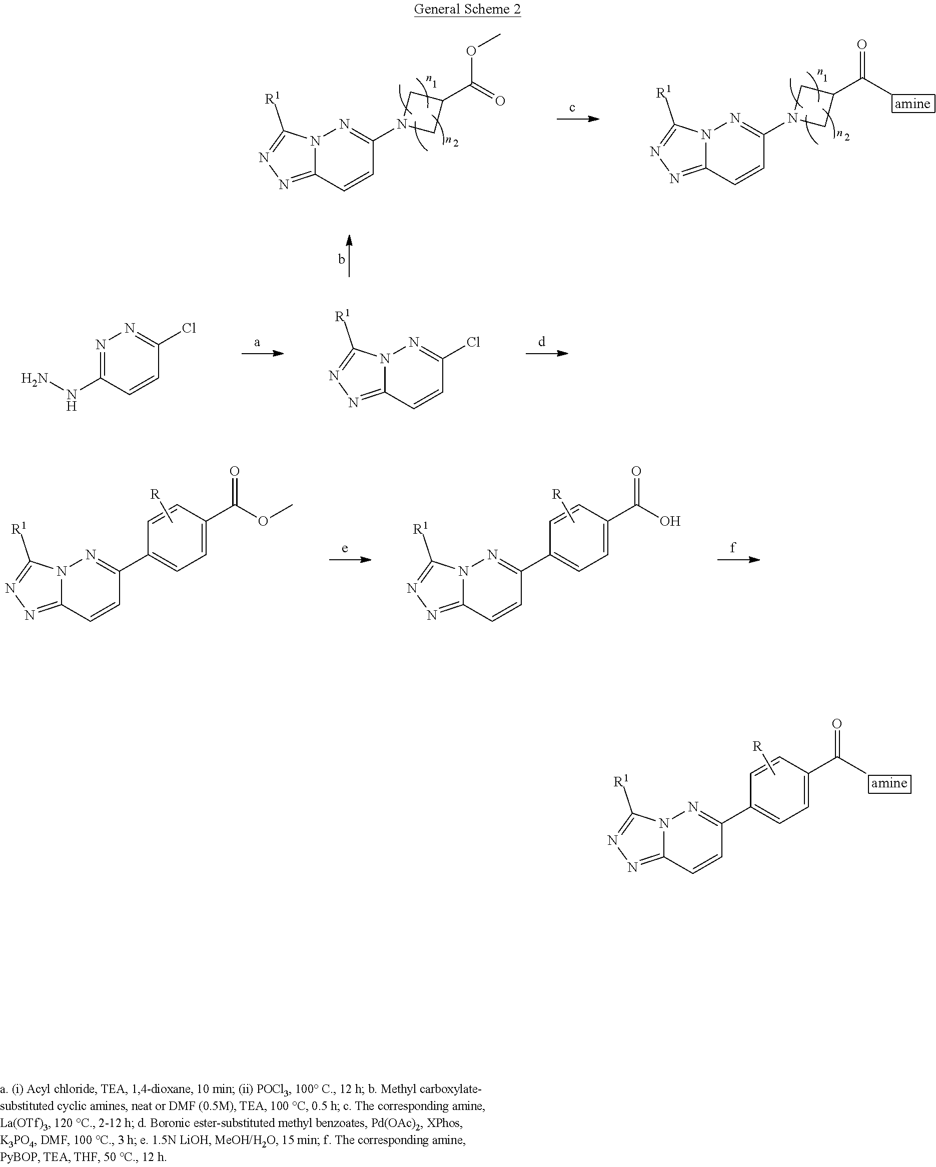

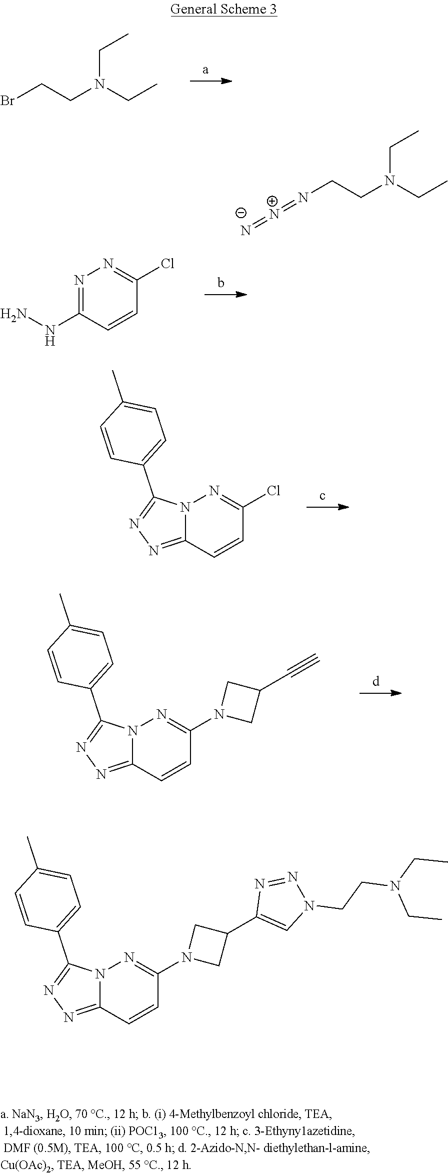

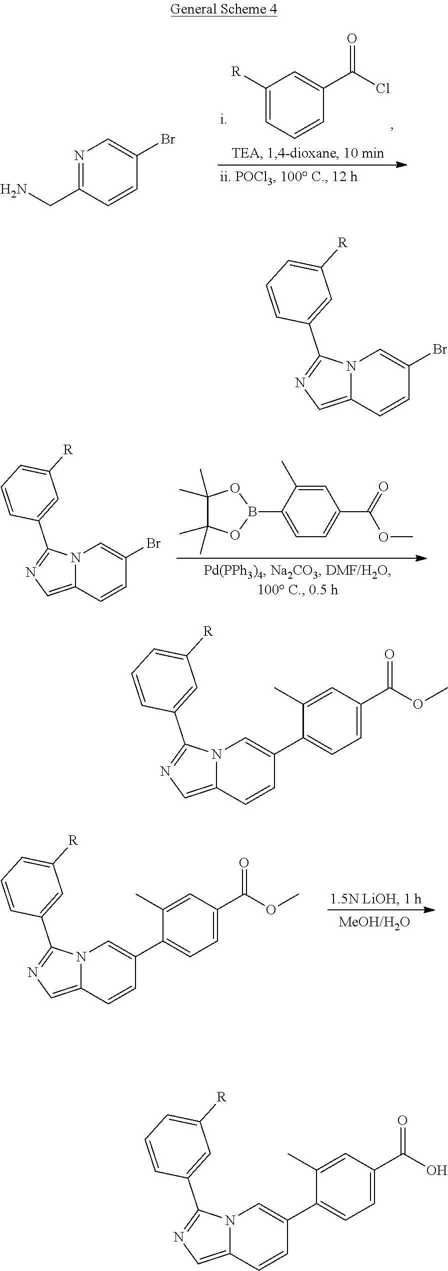

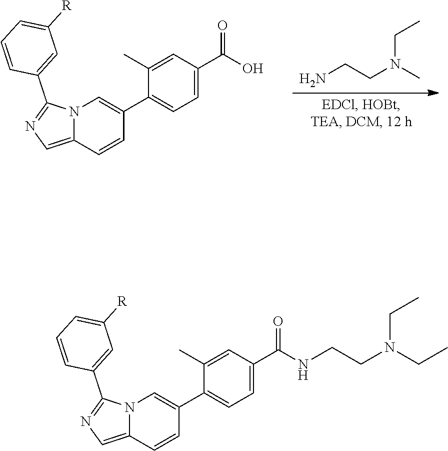

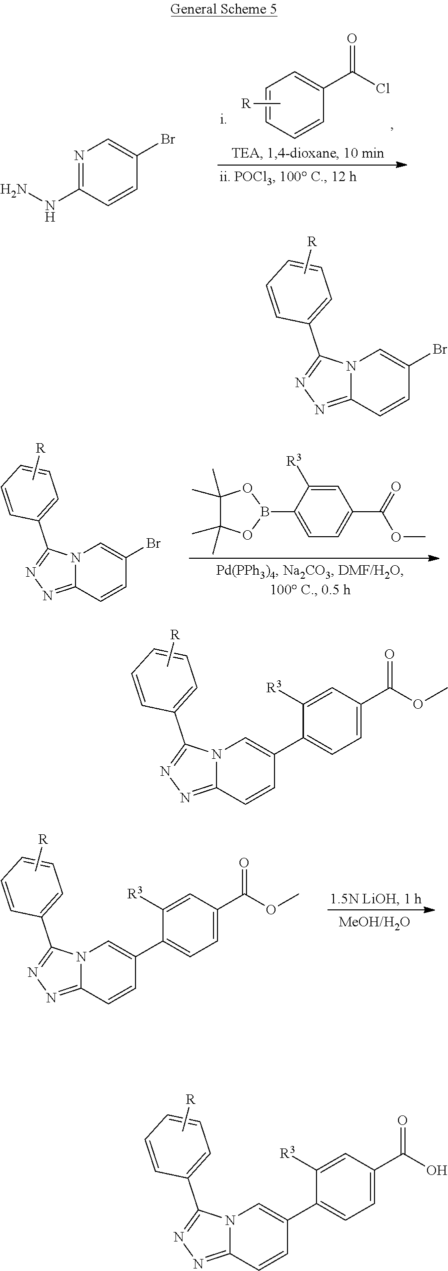

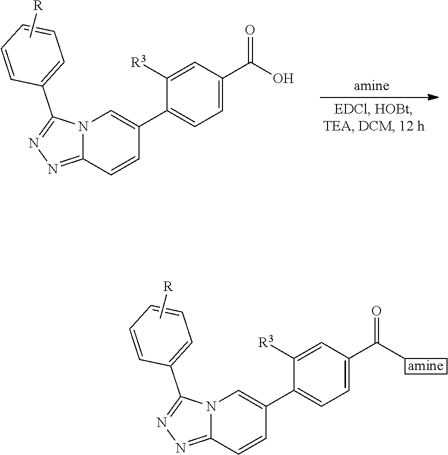

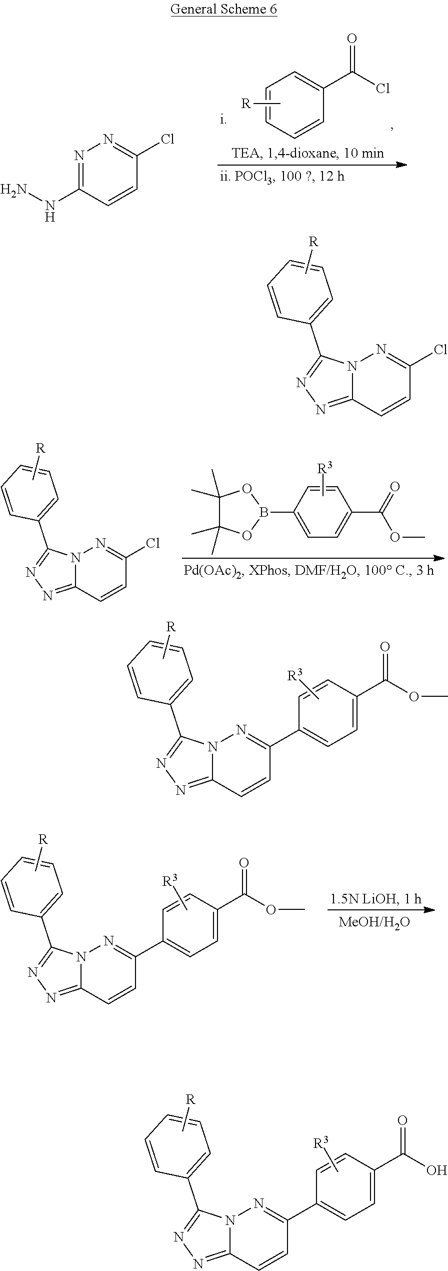

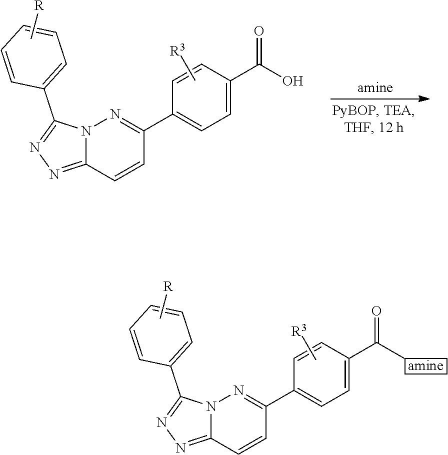

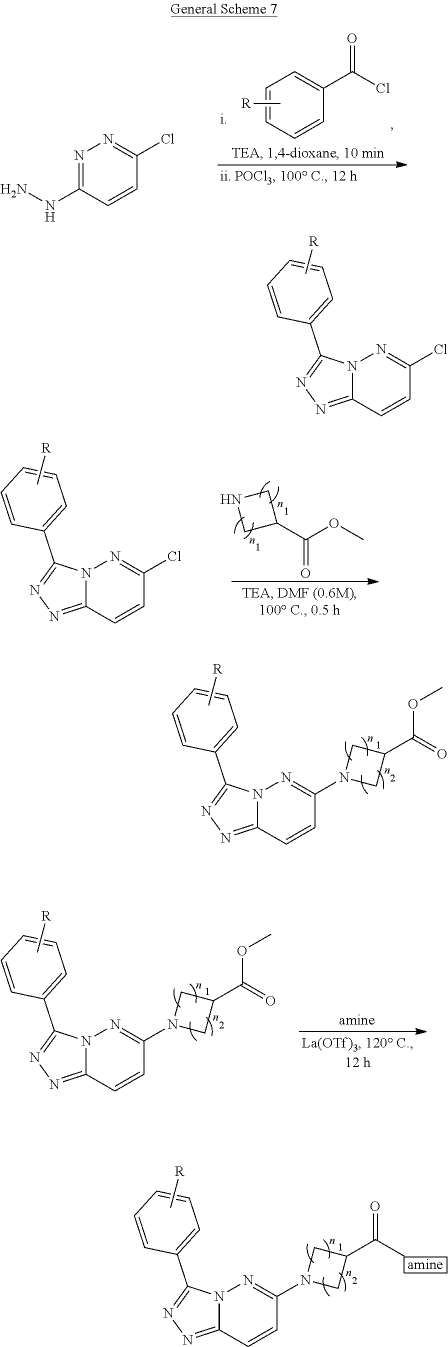

For Schemes 1-7 above:

In one embodiment the linker is the A group.

In one embodiment the —(C═O)amine moiety is R² (e.g., —C(═O)—NR^{a}R^{b}).

In one embodiment n₁ and n₂ are independently 0, 1, 2, 3, 4, 5, 6, or 7 (e.g., provided the ring is a 3-8 membered ring).

In one embodiment the R and R³ groups are independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy; or
selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

### General Procedure A:

1. To a solution of (5-bromopyridin-2-yl)methanamine (1.0 g, 5.3 mmol, 1 eq.) and triethylamine (2.1 mL, 16 mmol, 3 eq.) in 1,4-dioxane (20 mL), the corresponding acyl chloride (6.4 mmol, 1.2 eq.) was added dropwise. After 10 min of stirring, POCl₃ (1.6 mL, 16 mmol, 3.0 eq.) was added, and the mixture was stirred and heated overnight at 100° C. After the solution was cooled to room temperature, saturated NaHCO₃ solution was added dropwise to quench the reaction. The mixture was diluted with ethyl acetate (100 mL) and washed with water (2×80 mL) and brine (1×80 mL). The organic layer was dried over MgSO₄, concentrated under reduced pressure, and purified by CombiFlash chromatography (silica gel, hexanes/ethyl acetate, 1:2).
2. To a solution of 5-bromo-2-hydrazineylpyridine (1.0 g, 5.3 mmol, 1 eq.) and triethylamine (2.2 mL, 16 mmol, 3 eq.) in 1,4-dioxane (20 mL), the corresponding acyl chloride (6.4 mmol, 1.2 eq.) was added dropwise. After 10 min, POCl₃ (1.6 mL, 16 mmol, 3 eq.) was added, and the mixture was stirred and heated overnight at 100° C. After the solution was cooled to room temperature, saturated NaHCO₃ solution was added dropwise to quench the reaction. The mixture was diluted with ethyl acetate (100 mL) and washed with water (2×80 mL) and brine (1×80 mL). The organic layer was dried over MgSO₄, concentrated under reduced pressure, and purified by CombiFlash chromatography (silica gel, hexanes/ethyl acetate, 1:2).
3. To a solution of 3-chloro-6-hydrazineylpyridazine (1.0 g, 6.9 mmol, 1 eq.) and triethylamine (2.9 mL, 21 mmol, 3 eq.) in 1,4-dioxane (20 mL), the corresponding acyl chloride (11 mmol, 1.65 eq.) was added dropwise at 0° C. After 10 min, POCl₃ (2.0 mL, 21 mmol, 3 eq.) was added, and the mixture was stirred and heated overnight at 100° C. After the solution was cooled to room temperature, the saturated NaHCO₃ solution was added dropwise to quench the reaction. The mixture was diluted with ethyl acetate (100 mL) and washed with water (2×80 mL) and brine (1×80 mL). The organic layer was dried over MgSO₄, concentrated under reduced pressure, and purified by CombiFlash chromatography (silica gel, hexanes/ethyl acetate, 2:5).

### General Procedure B:

1. The substituted 6-bromo-3-phenylimidazo[1,5-a]pyridine (0.59 mmol, 1 eq.), the corresponding boronic acid pinacol ester (0.64 mmol, 1.1 eq.), Na₂CO₃ (186 mg, 1.8 mmol, 3 eq.), and Pd(PPh₃)₄ (54.2 mg, 0.047 mmol, 0.08 eq.) were added to a microwave vial and sealed with a metal cap with septum. The vial was evacuated and backfilled with N₂ three times. Then DMF/H₂O (4:1, 10 mL) was degassed and injected into the reaction mixture. The suspension was stirred and heated at 100° C. for 0.5 h. After cooling to room temperature, the solution was diluted with ethyl acetate (30 mL) and washed with water (2×30 mL) and brine (1×30 mL). The organic layer was dried over MgSO₄, concentrated under reduced pressure, and purified by CombiFlash chromatography (silica gel, hexanes/ethyl acetate, 1:3).
2. The substituted 6-bromo-3-phenyl-[1,2,4]triazolo[4,3-a]pyridine (0.59 mmol, 1 eq.), the corresponding boronic acid pinacol ester (0.64 mmol, 1.1 eq.), Na₂CO₃ (186 mg, 1.8 mmol, 3 eq.), and Pd(PPh₃)₄ (54.2 mg, 0.047 mmol, 0.08 eq.) were added to a microwave vial and sealed with a metal cap with septum. The vial was evacuated and backfilled with N₂ three times. Then DMF/H₂O (4:1, 10 mL) was degassed and injected into the reaction mixture. The suspension was stirred and heated at 100° C. for 0.5 h. After cooling to room temperature, the solution was diluted with ethyl acetate (30 mL) and washed with water (2×30 mL) and brine (1×30 mL). The organic layer was dried over MgSO₄, concentrated under reduced pressure, and purified by CombiFlash chromatography (silica gel, hexanes/ethyl acetate, 1:3).
3. The substituted 6-chloro-3-phenyl-[1,2,4]triazolo[4,3-b]pyridazine (0.82 mmol, 1 eq.), the corresponding boronic acid pinacol ester (0.98 mmol, 1.2 eq.), XPhos (172 mg, 0.37 mmol, 0.45 eq.), Pd(OAc)₂ (55.1 mg, 0.12 mmol, 0.15 eq.) and K₃PO₄ (347 mg, 1.6 mmol, 2 eq.) were added to a microwave vial and sealed with a metal cap with septum. The vial was evacuated and backfilled with N₂ three times. Then 1,4-dioxane/H₂O (5:1, 12 mL) was degassed and injected into the reaction mixture. The suspension was stirred and heated at 100° C. for 3-5 h. After cooling to room temperature, the solution was diluted with ethyl acetate (30 mL) and washed with water (2×30 mL) and brine (1×30 mL). The organic layer was dried over MgSO₄, concentrated under reduced pressure, and purified by CombiFlash chromatography (silica gel, hexanes/ethyl acetate, 1:4).
4. The substituted 6-bromo-3-phenylimidazo[1,5-a]pyridine (0.35 mmol, 1 eq.), the corresponding boronic acid pinacol ester (0.42 mmol, 1.2 eq.), SPhos (23 mg, 0.056 mmol, 0.16 eq.), Pd(OAc)₂ (6.3 mg, 0.028 mmol, 0.08 eq.) and K₃PO₄ (296 mg, 1.4 mmol, 4 eq.) were added to a microwave vial and sealed with a metal cap with septum. The vial was evacuated and backfilled with N₂ three times. Then H₂O (0.13 mL, 7.0 mmol, 20 eq.) and THF (4 mL) were degassed and injected into the reaction mixture. The suspension was stirred and heated at 90° C. for 0.5 h. After cooling to room temperature, the solution was diluted with ethyl acetate (30 mL) and washed with water (2×30 mL) and brine (1×30 mL). The organic layer was dried over MgSO₄, concentrated under reduced pressure, and purified by CombiFlash chromatography (silica gel, hexanes/ethyl acetate, 1:1).

### General Procedure C:

1. The substituted methyl 3-methyl-4-(3-phenylimidazo[1,5-a]pyridin-6-yl)benzoate (0.75 mmol, 1 eq.) was dissolved in MeOH (6 mL) and LiOH (4.5 M in H₂O, 3 mL) was added to the solution. The mixture was stirred and heated at 40° C. for 1 hour. 1 N HCl was added dropwise to the solution to adjust the pH to 6-7 until the product completely precipitated. The white solids were filtered and washed by water with vacuum filtration.
2. The substituted methyl 4-(3-phenyl-[1,2,4]triazolo[4,3-a]pyridin-6-yl)benzoate (0.75 mmol, 1 eq.) was dissolved in MeOH (6 mL) and LiOH (4.5 M in H₂O, 3 mL) was added to the solution. The mixture was stirred and heated at 40° C. for 1 hour. 1 N HCl was added dropwise to the solution to adjust the pH to 6-7 until the product completely precipitated. The white solids were filtered and washed by water with vacuum filtration.
3. The substituted methyl 4-(3-phenyl-[1,2,4]triazolo[4,3-b]pyridazin-6-yl)benzoate (0.76 mmol, 1 eq.) was dissolved in MeOH (6 mL), and LiOH (4.5 M in H₂O, 3 mL) was added to the solution. The mixture was stirred and heated at 45° C. for 1 hour. MeOH was removed under reduced pressure. 1 N HCl was added dropwise to the solution to adjust the pH to 6-7 until the product completely precipitated. The white solids were filtered and washed by water with vacuum filtration.
4. The methyl ester of 5-heteroaryl-imidazo[1,5-a]pyridines (0.35 mmol, 1 eq.) was dissolved in MeOH (3 mL) and LiOH (4.5 M in H₂O, 1.5 mL) was added to the solution. The mixture was stirred and heated at 40° C. for 1 hour. 1 N HCl was added dropwise to the solution to adjust the pH to 4-5 until the product completely precipitated. The white solids were filtered and washed by water with vacuum filtration.

### General Procedure D:

1. The substituted 4-(3-phenylimidazo[1,5-a]pyridin-6-yl)benzoic acid (0.11 mmol, 1 eq.), triethylamine (78 μL, 0.55 mmol, 5 eq.), EDCI (28 mg, 0.12 mmol, 1.1 eq.), and HOBt (19 mg, 0.12 mmol, 1.1 eq.) were dissolved in DCM (5 mL) and stirred for 10 min. The corresponding amine (31 μL, 0.22 mmol, 2 eq.) was then added to the solution, and the mixture was stirred at room temperature for 12 h. The mixture was diluted with DCM (15 mL) and washed with water (1×15 mL) and brine (1×15 mL). The organic layer was dried over MgSO₄, concentrated under reduced pressure, and purified by CombiFlash chromatography (silica gel, MeOH/CH₂Cl₂, 3:17).
2. The substituted 4-(3-phenylimidazo[1,5-a]pyridin-6-yl)benzoic acid (0.044 mmol, 1 eq.), triethylamine (31 μL, 0.22 mmol, 5 eq.), and HATU (18 mg, 0.049 mmol, 1.1 eq.) were dissolved in DMF (1.5 mL) and stirred for 20 min. The corresponding amine (0.088 mmol, 2 eq.) was then added to the solution, and the mixture was stirred at room temperature for 12 h. The reaction mixture was directly purified by CombiFlash chromatography (C18, ACN, 100%).
3. The carboxylic acid of 5-heteroaryl-imidazo[1,5-a]pyridines (0.075 mmol, 1 eq.), triethylamine (52 μL, 0.37 mmol, 5 eq.), and HATU (31 mg, 0.082 mmol, 1.1 eq.) were dissolved in DMF (1.5 mL) and stirred for 20 min. The corresponding amine (0.15 mmol, 2 eq.) was then added to the solution, and the mixture was stirred at room temperature for 12 h. The reaction mixture was directly purified by prepHPLC (ACN/H₂O, 9:1).
4. The substituted 4-(3-phenyl-[1,2,4]triazolo[4,3-a]pyridin-6-yl)benzoic acid (0.11 mmol, 1 eq.), triethylamine (78 μL, 0.55 mmol, 5 eq.), EDCI (28 mg, 0.12 mmol, 1.1 eq.), and HOBt (19 mg, 0.12 mmol, 1.1 eq.) were dissolved in DCM (5 mL) and stirred for 10 min. N¹,N¹-diethylethane-1,2-diamine (31 μL, 0.22 mmol, 2 eq.) was then added to the solution, and the mixture was stirred at room temperature for 12 h. The mixture was diluted with DCM (15 mL) and washed with water (1×15 mL) and brine (1×15 mL). The organic layer was dried over MgSO₄, concentrated under reduced pressure, and purified by CombiFlash chromatography (silica gel, MeOH/CH₂Cl₂, 3:17).
5. The substituted 4-(3-phenyl-[1,2,4]triazolo[4,3-b]pyridazin-6-yl)benzoic acid (0.12 mmol, 1 eq.), triethylamine (49 μL, 0.35 mmol, 3 eq.), and PyBOP (73 mg, 0.14 mmol, 1.2 eq.) were dissolved in THF (5 mL) and stirred for 10 min. The corresponding amine (0.23 mmol, 2 eq.) was added to the solution, and the mixture was stirred at room temperature for 12 h. The solution was diluted with ethyl acetate (15 mL) and washed with water (1×15 mL) and brine (1×15 mL). The organic layer was dried over MgSO₄, concentrated under reduced pressure, and purified by CombiFlash chromatography (silica gel, MeOH/CH₂Cl₂, 1:4).

### General Procedure E:

1. The HCl salt of the corresponding amine (3.9 mmol, 5 eq.) was dissolved in DMF (6.5 mL). The substituted 6-chloro-3-phenyl-[1,2,4]triazolo[4,3-b]pyridazine (0.61 mmol, 1 eq.) and triethylamine (911 μL, 6.5 mmol, 8 eq.) were added to the suspension. The mixture was stirred and heated at 110° C. for 0.5 h. The suspension was cooled to room temperature, diluted with ethyl acetate, and washed with water (2×30 mL) and brine (1×30 mL). The organic layer was dried over MgSO₄, concentrated under reduced pressure, and purified by CombiFlash chromatography (silica gel, MeOH/CH₂Cl₂, 1:19).
2. The substituted 6-chloro-3-phenyl-[1,2,4]triazolo[4,3-b]pyridazine (0.61 mmol, 1 eq.) was dissolved in the corresponding amine (2.5 mL). The mixture was stirred and heated at 110° C. for 0.5 h. The solution was cooled to temperature, and hexanes (2 mL) were added to the suspension to completely precipitate the product. The white solids were filtered, washed with hexanes, dried, and collected.

### General Procedure F:

The corresponding ester (0.26 mmol, 1 eq.) and La(OTf)₃ (0.026 mmol, 0.1 eq.) was added to an oven-dried microwave vial and sealed with a metal cap with septum. The vial was evacuated and backfilled with N₂ three times. Then, the amine (1 mL) was degassed and injected into the reaction mixture. The suspension was heated and stirred at 120° C. for 12 h. After cooling to room temperature, the mixture was diluted with 3 mL MeOH and directly purified by CombiFlash chromatography (C18, ACN/Water, 1:1).

### Preparations

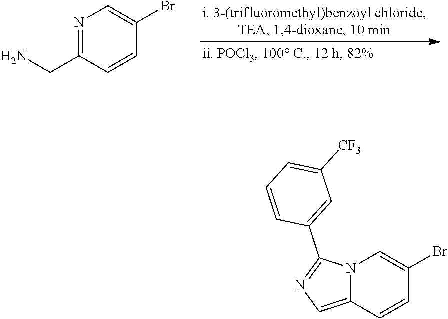 [SMILES: NCc1ccc(Br)cn1 ; FC(F)(F)c1cccc(-c2ncc3ccc(Br)cn23)c1]

6-Bromo-3-(3-(trifluoromethyl)phenyl)imidazo[1,5-a]pyridine: General procedure A1 was employed using 3-(trifluoromethyl)benzoyl chloride (968 μL, 6.4 mmol, 1.2 eq.). A yellow solid (1.5 g, 82%) was isolated by CombiFlash chromatography (silica gel, hexanes/ethyl acetate, 1:2). ¹H NMR (400 MHZ, CDCl₃) δ 8.30 (d, J=3.0 Hz, 1H), 8.07 (s, 2H), 8.00 (d, J=7.9 Hz, 1H), 7.77 (d, J=8.8 Hz, 1H), 7.70 (dd, J=8.4, 2.9 Hz, 1H), 7.58 (t, J=8.1 Hz, 1H), 7.29 (d, J=8.8 Hz, 1H).

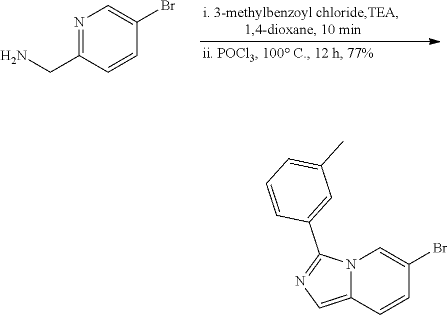 [SMILES: NCc1ccc(Br)cn1 ; Cc1cccc(-c2ncc3ccc(Br)cn23)c1]

6-Bromo-3-(3-tolyl)imidazo[1,5-a]pyridine: General procedure A1 was employed using 3-methylbenzoyl chloride (373 μL, 2.6 mmol, 1.2 eq.). A yellow solid (0.47 g, 77%) was isolated by CombiFlash chromatography (silica gel, hexanes/ethyl acetate, 1:2). ¹H NMR (400 MHZ, CDCl₃) δ 8.38 (s, 1H), 7.62-7.51 (m, 3H), 7.43 (t, J=7.6 Hz, 1H), 7.38 (dd, J=9.5, 1.0 Hz, 1H), 7.28 (d, J=7.3 Hz, 1H), 6.76 (dd, J=9.5, 1.5 Hz, 1H), 2.45 (s, 3H).

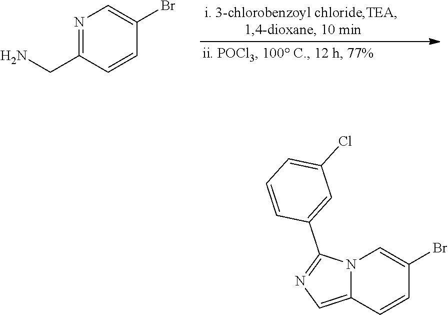 [SMILES: NCc1ccc(Br)cn1 ; Clc1cccc(-c2ncc3ccc(Br)cn23)c1]

6-Bromo-3-(3-chlorophenyl)imidazo[1,5-a]pyridine: General procedure A1 was employed using 3-chlorobenzoyl chloride (821 μL, 6.4 mmol, 1.2 eq.). A yellow solid (1.3 g, 77%) was isolated by CombiFlash chromatography (silica gel, hexanes/ethyl acetate, 1:2). ¹H NMR (400 MHZ, CDCl₃) δ 8.37 (q, J=1.1 Hz, 1H), 7.79 (t, J=1.7 Hz, 1H), 7.66 (dt, J=7.5, 1.5 Hz, 1H), 7.59 (d, J=1.0 Hz, 1H), 7.51-7.39 (m, 3H), 6.82 (dd, J=9.5, 1.5 Hz, 1H).

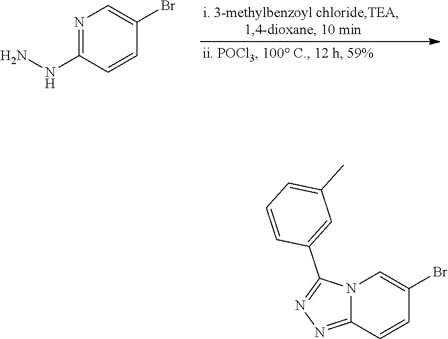 [SMILES: NNc1ccc(Br)cn1 ; Cc1cccc(-c2nnc3ccc(Br)cn23)c1]

6-Bromo-3-(3-tolyl)-[1,2,4]triazolo[4,3-a]pyridine: General procedure A2 was employed using 3-methylbenzoyl chloride (841 μL, 6.4 mmol, 1.2 eq.). A light-yellow solid (0.90 g, 59%) was isolated by CombiFlash chromatography (silica gel, hexanes/ethyl acetate, 1:2). ¹H NMR (400 MHz, CDCl₃) δ 8.48-8.37 (m, 1H), 7.76 (dd, J=9.7, 1.0 Hz, 1H), 7.67 (s, 1H), 7.60 (d, J=7.7 Hz, 1H), 7.52 (t, J=7.6 Hz, 1H), 7.42 (d, J=7.6 Hz, 1H), 7.35 (dd, J=9.7, 1.7 Hz, 1H), 2.51 (s, 3H).

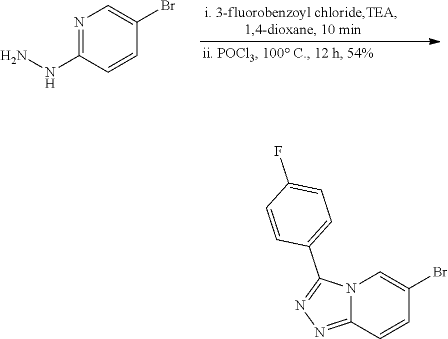 [SMILES: NNc1ccc(Br)cn1 ; Fc1ccc(-c2nnc3ccc(Br)cn23)cc1]

6-Bromo-3-(4-fluorophenyl)-[1,2,4]triazolo[4,3-a]pyridine: General procedure A2 was employed using 4-fluorobenzoyl chloride (754 μL, 6.4 mmol, 1.2 eq.). A white solid (0.80 g, 54%) was isolated by CombiFlash chromatography (silica gel, hexanes/ethyl acetate, 1:2). ¹H NMR (400 MHz, CDCl₃) δ 8.35 (dd, J=1.7, 1.0 Hz, 1H), 7.85-7.79 (m, 2H), 7.75 (dd, J=9.7, 1.0 Hz, 1H), 7.38-7.29 (m, 3H).

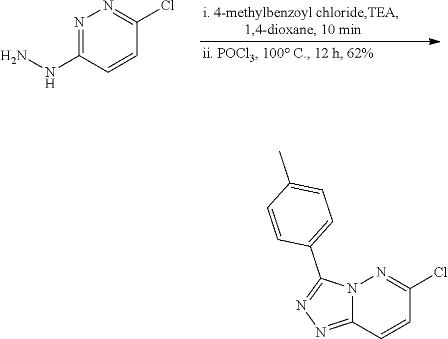 [SMILES: NNc1ccc(Cl)nn1 ; Cc1ccc(-c2nnc3ccc(Cl)nn23)cc1]

6-Chloro-3-(4-tolyl)-[1,2,4]triazolo[4,3-b]pyridazine: General procedure A3 was employed using 4-methylbenzoyl chloride (1.51 mL, 11 mmol, 1.65 eq.). A gray solid (1.1 g, 62%) was isolated by CombiFlash chromatography (silica gel, hexanes/ethyl acetate, 2:5). ¹H NMR (400 MHz, CDCl₃) δ 8.35 (d, J=8.3 Hz, 2H), 8.14 (d, J=9.6 Hz, 1H), 7.38 (d, J=8.0 Hz, 2H), 7.13 (d, J=9.6 Hz, 1H), 2.46 (s, 3H).

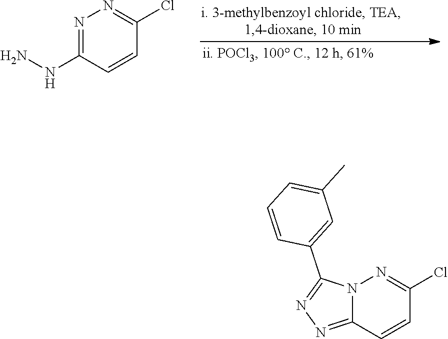 [SMILES: NNc1ccc(Cl)nn1 ; Cc1cccc(-c2nnc3ccc(Cl)nn23)c1]

6-Chloro-3-(3-tolyl)-[1,2,4]triazolo[4,3-b]pyridazine: General procedure A3 was employed using 3-methylbenzoyl chloride (250 μL, 1.7 mmol, 1.65 eq.). A light-yellow solid (0.15 g, 61%) was isolated by CombiFlash chromatography (silica gel, hexanes/ethyl acetate, 2:5). ¹H NMR (400 MHZ, CDCl₃) δ 8.31-8.27 (m, 2H), 8.17 (d, J=9.6 Hz, 1H), 7.49 (t, J=8.0 Hz, 1H), 7.38 (d, J=7.7 Hz, 1H), 7.17 (d, J=9.6 Hz, 1H), 2.51 (s, 3H).

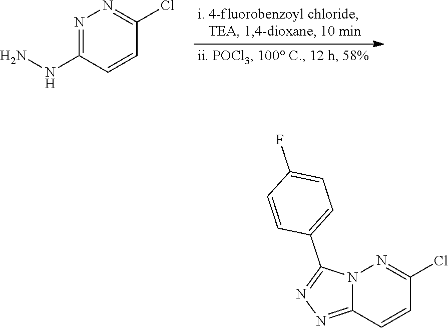 [SMILES: NNc1ccc(Cl)nn1 ; Fc1ccc(-c2nnc3ccc(Cl)nn23)cc1]

6-Chloro-3-(4-fluorophenyl)-[1,2,4]triazolo[4,3-b]pyridazine): General procedure A3 was employed using 4-fluorobenzoyl chloride (1.08 mL, 9.1 mmol, 1.65 eq.). A white solid (0.80 g, 58%) was isolated by CombiFlash chromatography (silica gel, hexanes/ethyl acetate, 2:5). ¹H NMR (400 MHZ, CDCl₃) δ 8.53-8.47 (m, 2H), 8.16 (d, J=9.6 Hz, 1H), 7.30-7.27 (m, 1H), 7.26 (d, J=2.5 Hz, 1H), 7.17 (d, J=9.6 Hz, 1H).

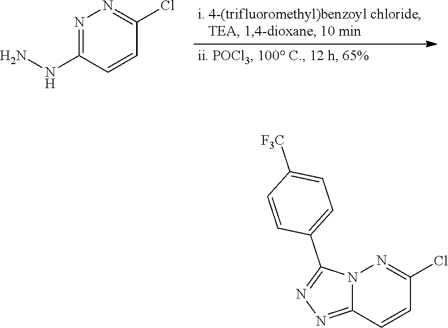 [SMILES: NNc1ccc(Cl)nn1 ; FC(F)(F)c1ccc(-c2nnc3ccc(Cl)nn23)cc1]

6-Chloro-3-(4-(trifluoromethyl)phenyl)-[1,2,4]triazolo[4,3-b]pyridazine: General procedure A3 was employed using 4-(trifluoromethyl)benzoyl chloride (1.36 mL, 9.1 mmol, 1.65 eq.). A white solid (1.1 g, 65%) was isolated by CombiFlash chromatography (silica gel, hexanes/ethyl acetate, 2:5). ¹H NMR (400 MHZ, CDCl₃) δ 8.64 (d, J=7.8 Hz, 2H), 8.20 (d, J=9.6 Hz, 1H), 7.85 (d, J=8.2 Hz, 2H), 7.22 (d, J=9.6 Hz, 1H).

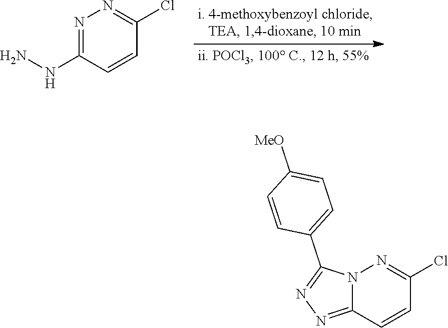 [SMILES: NNc1ccc(Cl)nn1 ; COc1ccc(-c2nnc3ccc(Cl)nn23)cc1]

6-Chloro-3-(4-methoxyphenyl)-[1,2,4]triazolo[4,3-b]pyridazine: General procedure A3 was employed using 4-methoxybenzoyl chloride (0.773 mL, 5.7 mmol, 1.65 eq.). A white solid (0.50 g, 55%) was isolated by CombiFlash chromatography (silica gel, hexanes/ethyl acetate, 2:5). The product was used for the next step without further characterization.

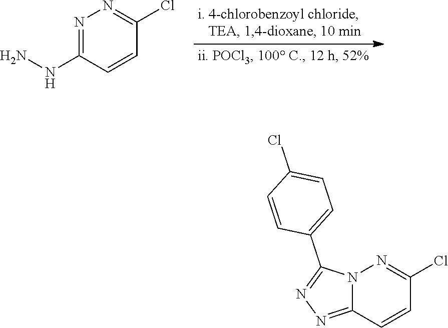 [SMILES: NNc1ccc(Cl)nn1 ; Clc1ccc(-c2nnc3ccc(Cl)nn23)cc1]

6-Chloro-3-(4-chlorophenyl)-[1,2,4]triazolo[4,3-b]pyridazine): General procedure A3 was employed using 4-chlorobenzoyl chloride (1.17 mL, 9.1 mmol, 1.65 eq.). A white solid (0.76 g, 52%) was isolated by CombiFlash chromatography (silica gel, hexanes/ethyl acetate, 2:5). ¹H NMR (400 MHZ, CDCl₃) δ 8.45 (d, J=8.7 Hz, 2H), 8.17 (d, J=9.6 Hz, 1H), 7.56 (d, J=8.7 Hz, 2H), 7.18 (d, J=9.6 Hz, 1H).

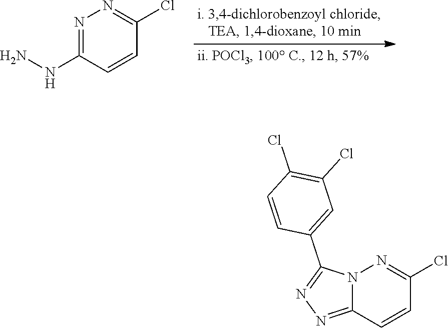 [SMILES: NNc1ccc(Cl)nn1 ; Clc1ccc2nnc(-c3ccc(Cl)c(Cl)c3)n2n1]

6-Chloro-3-(3,4-dichlorophenyl)-[1,2,4]triazolo[4,3-b]pyridazine: General procedure A3 was employed using 3,4-dichlorobenzoyl chloride (853 μL, 5.8 mmol, 1.65 eq.). A white solid (0.59 g, 57%) was isolated by CombiFlash chromatography (silica gel, hexanes/ethyl acetate, 2:5). ¹H NMR (400 MHZ, CDCl₃) δ 8.65 (d, J=2.0 Hz, 1H), 8.38 (dd, J=8.5, 2.1 Hz, 1H), 8.19 (d, J=9.6 Hz, 1H), 7.66 (d, J=8.4 Hz, 1H), 7.21 (d, J=9.6 Hz, 1H).

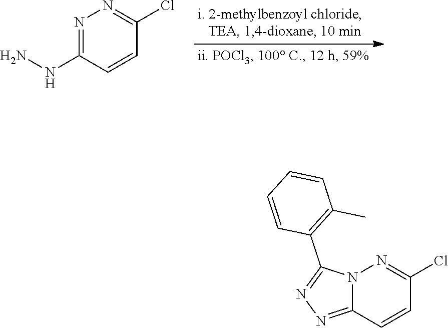 [SMILES: NNc1ccc(Cl)nn1 ; Cc1ccccc1-c1nnc2ccc(Cl)nn12]

6-Chloro-3-(2-tolyl)-[1,2,4]triazolo[4,3-b]pyridazine: General procedure A3 was employed using 2-methylbenzoyl chloride (750 μL, 5.8 mmol, 1.65 eq.). A white solid (0.50 g, 59%) was isolated by CombiFlash chromatography (silica gel, hexanes/ethyl acetate, 2:5). ¹H NMR (400 MHz, CDCl₃) δ 8.16 (d, J=9.6 Hz, 1H), 7.67 (dd, J=7.7, 1.4 Hz, 1H), 7.48 (td, J=7.5, 1.5 Hz, 1H), 7.44-7.34 (m, 2H), 7.16 (d, J=9.6 Hz, 1H), 2.40 (s, 3H).

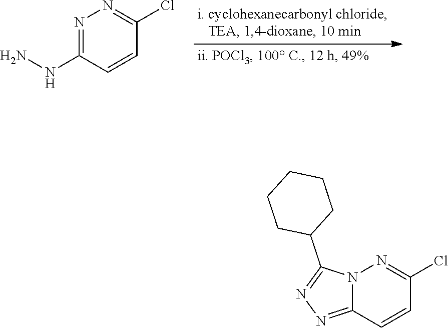 [SMILES: NNc1ccc(Cl)nn1 ; Clc1ccc2nnc(C3CCCCC3)n2n1]

6-Chloro-3-cyclohexyl-[1,2,4]triazolo[4,3-b]pyridazine: General procedure A3 was employed using cyclohexanecarbonyl chloride (620 μL, 4.6 mmol, 1.65 eq.). A white solid (0.32 g, 49%) was isolated by CombiFlash chromatography (silica gel, hexanes/ethyl acetate, 2:5). ¹H NMR (400 MHZ, CDCl₃) δ 8.05 (d, J=9.6 Hz, 1H), 7.07 (d, J=9.6 Hz, 1H), 3.33 (tt, J=11.6, 3.6 Hz, 1H), 2.17-2.08 (m, 2H), 1.97-1.74 (m, 6H), 1.51-1.38 (m, 2H).

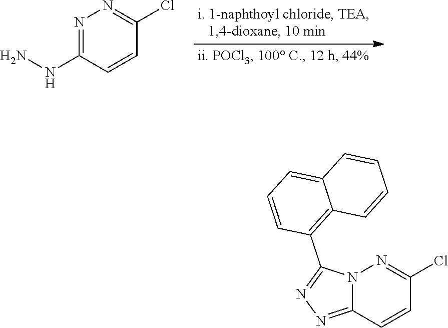 [SMILES: NNc1ccc(Cl)nn1 ; Clc1ccc2nnc(-c3cccc4ccccc34)n2n1]

6-Chloro-3-(naphthalen-1-yl)-[1,2,4]triazolo[4,3-b]pyridazine: General procedure A3 was employed using 1-naphthoyl chloride (688 μL, 4.6 mmol, 1.65 eq.). A white solid (0.34 g, 44%) was isolated by CombiFlash chromatography (silica gel, hexanes/ethyl acetate, 2:5). ¹H NMR (400 MHZ, CDCl₃) δ 8.26-8.18 (m, 2H), 8.08 (d, J=8.1 Hz, 1H), 8.02-7.94 (m, 2H), 7.67 (dd, J=8.3, 7.2 Hz, 1H), 7.60-7.52 (m, 2H), 7.19 (d, J=9.6 Hz, 1H).

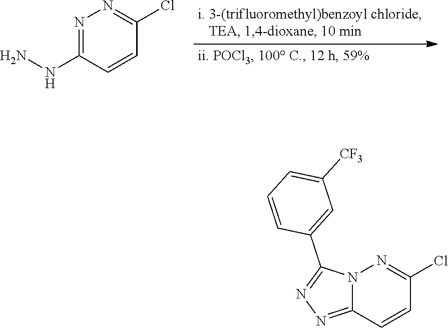 [SMILES: NNc1ccc(Cl)nn1 ; FC(F)(F)c1cccc(-c2nnc3ccc(Cl)nn23)c1]

6-Chloro-3-(3-(trifluoromethyl)phenyl)-[1,2,4]triazolo[4,3-b]pyridazine: General procedure A3 was employed using 3-(trifluoromethyl)benzoyl chloride (516 μL, 3.4 mmol, 1.65 eq.). A white solid (0.37 g, 59%) was isolated by CombiFlash chromatography (silica gel, hexanes/ethyl acetate, 2:5). ¹H NMR (400 MHZ, CDCl₃) δ 8.80 (s, 1H), 8.71 (d, J=7.9 Hz, 1H), 8.20 (d, J=9.6 Hz, 1H), 7.86-7.77 (m, 1H), 7.72 (t, J=7.9 Hz, 1H), 7.22 (d, J=9.6 Hz, 1H).

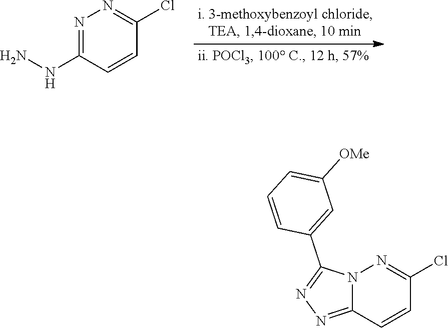 [SMILES: NNc1ccc(Cl)nn1 ; COc1cccc(-c2nnc3ccc(Cl)nn23)c1]

6-Chloro-3-(3-methoxyphenyl)-[1,2,4]triazolo[4,3-b]pyridazine: General procedure A3 was employed using 3-methoxybenzoyl chloride (321 μL, 2.3 mmol, 1.65 eq.). A white solid (0.21 g, 57%) was isolated by CombiFlash chromatography (silica gel, hexanes/ethyl acetate, 2:5). ¹H NMR (400 MHZ, CDCl₃) δ 8.16 (d, J=9.6 Hz, 1H), 8.09 (ddd, J=7.8, 1.6, 1.0 Hz, 1H), 8.03 (dd, J=2.6, 1.6 Hz, 1H), 7.49 (t, J=8.0 Hz, 1H), 7.16 (d, J=9.6 Hz, 1H), 7.09 (ddd, J=8.4, 2.7, 1.0 Hz, 1H), 3.93 (s, 3H).

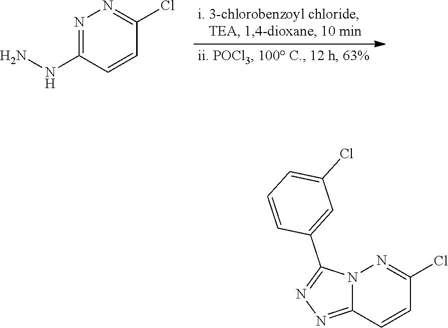 [SMILES: NNc1ccc(Cl)nn1 ; Clc1cccc(-c2nnc3ccc(Cl)nn23)c1]

6-Chloro-3-(3-chlorophenyl)-[1,2,4]triazolo[4,3-b]pyridazine: General procedure A3 was employed using 3-chlorobenzoyl chloride (292 μL, 2.3 mmol, 1.65 eq.). A white solid (0.23 g, 63%) was isolated by CombiFlash chromatography (silica gel, hexanes/ethyl acetate, 2:5). ¹H NMR (400 MHZ, CDCl₃) δ 8.51 (td, J=1.6, 0.9 Hz, 1H), 8.44-8.36 (m, 1H), 8.18 (d, J=9.6 Hz, 1H), 7.56-7.49 (m, 2H), 7.20 (d, J=9.7 Hz, 1H).

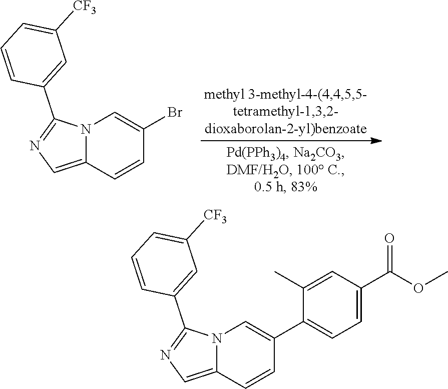 [SMILES: FC(F)(F)c1cccc(-c2ncc3ccc(Br)cn23)c1 ; COC(=O)c1ccc(-c2ccc3cnc(-c4cccc(C(F)(F)F)c4)n3c2)c(C)c1]

Methyl 3-Methyl-4-(3-(3-(trifluoromethyl)phenyl)imidazo[1,5-a]pyridin-6-yl)benzoate: General procedure B1 was employed using 6-bromo-3-(3-(trifluoromethyl)phenyl)-imidazo[1,5-a]pyridine (200 mg, 0.59 mmol, 1 eq.). A yellow solid (0.20 g, 83%) was isolated by CombiFlash chromatography (silica gel, hexanes/ethyl acetate, 1:3). ¹H NMR (400 MHZ, CDCl₃) δ 8.45 (s, 1H), 8.18 (s, 1H), 8.06 (d, J=7.2 Hz, 1H), 7.93 (d, J=8.0 Hz, 1H), 7.76-7.62 (m, 4H), 7.17 (dd, J=8.0, 1.7 Hz, 1H), 7.13 (d, J=1.6 Hz, 1H), 7.09 (dd, J=9.4, 1.4 Hz, 1H), 4.00 (s, 3H), 3.94 (s, 3H).

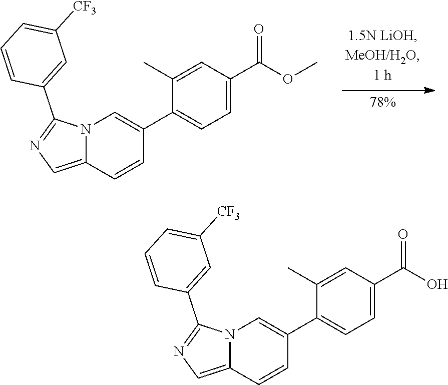 [SMILES: COC(=O)c1ccc(-c2ccc3cnc(-c4cccc(C(F)(F)F)c4)n3c2)c(C)c1 ; Cc1cc(C(=O)O)ccc1-c1ccc2cnc(-c3cccc(C(F)(F)F)c3)n2c1]

3-Methyl-4-(3-(3-(trifluoromethyl)phenyl)imidazo[1,5-a]pyridin-6-yl)benzoic Acid: General procedure C1 was employed using methyl 3-methyl-4-(3-(3-(trifluoromethyl)phenyl)imidazo[1,5-a]pyridin-6-yl)benzoate (200 mg, 0.49 mmol, 1 eq.). A white solid (150 mg, 78%) was isolated through filtration. ¹H NMR (400 MHZ, DMSO-d₆) δ 8.38 (s, 1H), 8.22 (d, J=7.4 Hz, 1H), 8.17 (s, 1H), 7.91 (d, J=1.8 Hz, 1H), 7.86-7.73 (m, 4H), 7.71 (s, 1H), 7.50 (d, J=7.9 Hz, 1H), 6.98 (d, J=9.3 Hz, 1H), 2.38 (s, 3H).

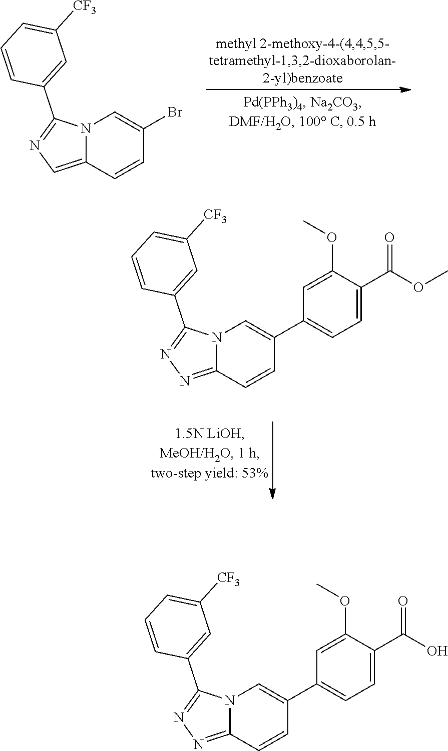 [SMILES: FC(F)(F)c1cccc(-c2ncc3ccc(Br)cn23)c1 ; COC(=O)c1ccc(-c2ccc3nnc(-c4cccc(C(F)(F)F)c4)n3c2)cc1OC ; COc1cc(-c2ccc3nnc(-c4cccc(C(F)(F)F)c4)n3c2)ccc1C(=O)O]

2-Methoxy-4-(3-(3-(trifluoromethyl)phenyl)imidazo[1,5-a]pyridin-6-yl)benzoic Acid: General procedure B1 was employed using 6-bromo-3-(3-(trifluoromethyl)phenyl)-imidazo[1,5-a]pyridine (200 mg, 0.59 mmol, 1 eq.). The intermediate product was isolated by CombiFlash chromatography (silica gel, hexanes/ethyl acetate, 1:1) and hydrolyzed as described in General procedure C1, which led to a yellow solid (0.19 g, 53%). ¹H NMR (400 MHZ, MeOD) δ 8.61 (s, 1H), 8.28-8.13 (m, 2H), 7.92 (dd, J=8.0, 1.7 Hz, 1H), 7.87-7.81 (m, 2H), 7.79 (d, J=9.6 Hz, 1H), 7.66 (s, 1H), 7.39 (s, 1H), 7.35-7.25 (m, 2H), 4.00 (s, 3H).

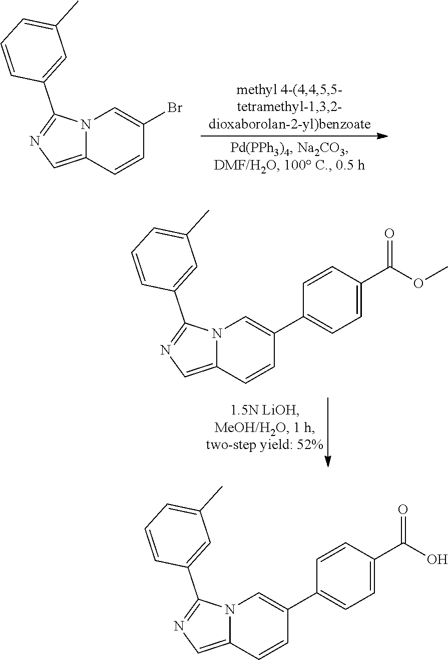 [SMILES: Cc1cccc(-c2ncc3ccc(Br)cn23)c1 ; COC(=O)c1ccc(-c2ccc3cnc(-c4cccc(C)c4)n3c2)cc1 ; Cc1cccc(-c2ncc3ccc(-c4ccc(C(=O)O)cc4)cn23)c1]

4-(3-(3-Tolyl)imidazo[1,5-a]pyridin-6-yl)benzoic Acid: General procedure B1 was employed using 6-bromo-3-(3-tolyl)imidazo[1,5-a]pyridine (500 mg, 1.7 mmol, 1 eq.). The intermediate product was isolated by CombiFlash chromatography (silica gel, hexanes/ethyl acetate, 1:1) and hydrolyzed as described in General procedure C1, which led to a yellow solid (0.40 g, 52%). ¹H NMR (400 MHZ, DMSO-d₆) δ 8.54 (s, 1H), 8.01 (d, J=7.9 Hz, 2H), 7.84-7.74 (m, 3H), 7.71-7.64 (m, 2H), 7.59 (s, 1H), 7.46 (t, J=7.7 Hz, 1H), 7.30 (d, J=7.7 Hz, 1H), 7.21 (d, J=9.5 Hz, 1H), 2.40 (s, 3H).

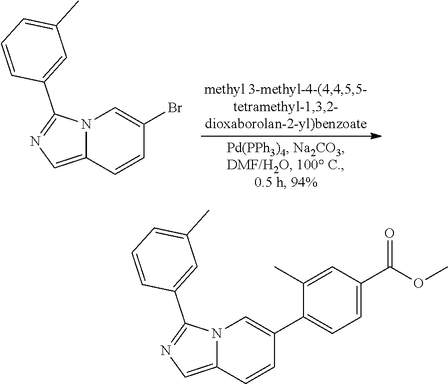 [SMILES: Cc1cccc(-c2ncc3ccc(Br)cn23)c1 ; COC(=O)c1ccc(-c2ccc3cnc(-c4cccc(C)c4)n3c2)c(C)c1]

Methyl 3-Methyl-4-(3-(3-methylphenyl)imidazo[1,5-a]pyridin-6-yl)benzoate: General procedure B1 was employed using 6-bromo-3-(3-tolyl)imidazo[1,5-a]pyridine (600 mg, 2.1 mmol, 1 eq.). A yellow solid (0.70 g, 94%) was isolated by CombiFlash chromatography (silica gel, hexanes/ethyl acetate, 1:3). ¹H NMR (400 MHZ, DMSO-d₆) δ 8.06 (s, 2H), 7.87 (d, J=8.5 Hz, 2H), 7.79 (dd, J=9.4, 1.1 Hz, 1H), 7.75-7.68 (m, 2H), 7.60 (s, 1H), 7.46 (t, J=7.6 Hz, 1H), 7.31 (d, J=7.6 Hz, 1H), 7.23 (dd, J=9.5, 1.5 Hz, 1H), 3.88 (s, 3H), 3.30 (s, 3H), 2.42 (s, 3H).

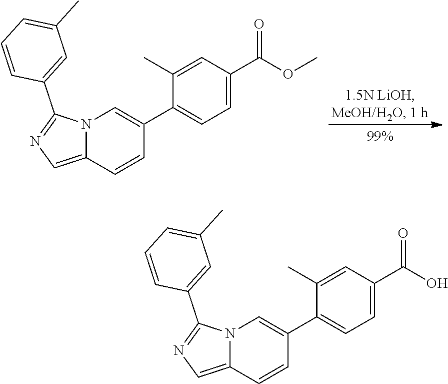 [SMILES: COC(=O)c1ccc(-c2ccc3cnc(-c4cccc(C)c4)n3c2)c(C)c1 ; Cc1cccc(-c2ncc3ccc(-c4ccc(C(=O)O)cc4C)cn23)c1]

3-Methyl-4-(3-(3-tolyl)imidazo[1,5-a]pyridin-6-yl)benzoic Acid: General procedure C1 was employed using methyl 3-methyl-4-(3-(3-tolyl)imidazo[1,5-a]pyridin-6-yl)benzoate (700 mg, 1.96 mmol, 1 eq.). A white solid (670 mg, 99%) was isolated through filtration without further characterization.

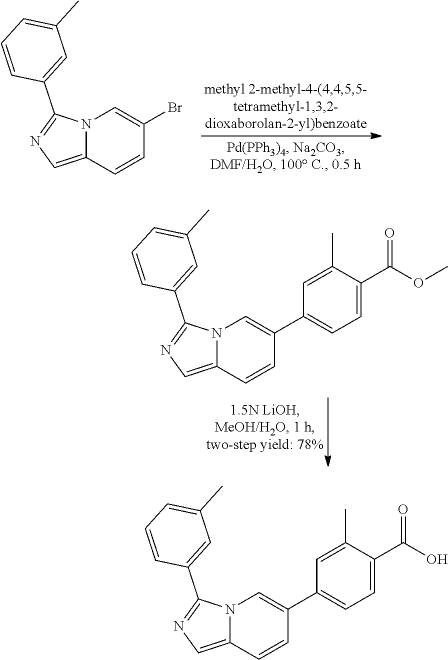 [SMILES: Cc1cccc(-c2ncc3ccc(Br)cn23)c1 ; COC(=O)c1ccc(-c2ccc3cnc(-c4cccc(C)c4)n3c2)cc1C ; Cc1cccc(-c2ncc3ccc(-c4ccc(C(=O)O)c(C)c4)cn23)c1]

2-Methyl-4-(3-(3-tolyl)imidazo[1,5-a]pyridin-6-yl)benzoic Acid: General procedure B1 was employed using 6-bromo-3-(3-tolyl)imidazo[1,5-a]pyridine (250 mg, 0.87 mmol, 1 eq.). The intermediate product was isolated by CombiFlash chromatography (silica gel, hexanes/ethyl acetate, 1:1) and hydrolyzed as described in General procedure C1, which led to a yellow solid (0.23 g, 78%). ¹H NMR (400 MHZ, DMSO-d₆) δ 8.51 (s, 1H), 7.90 (d, J=8.1 Hz, 1H), 7.76 (d, J=9.4 Hz, 1H), 7.70-7.64 (m, 2H), 7.63-7.54 (m, 3H), 7.46 (t, J=7.6 Hz, 1H), 7.30 (d, J=7.6 Hz, 1H), 7.20 (dd, J=9.4, 1.4 Hz, 1H), 2.57 (s, 3H), 2.40 (s, 3H).

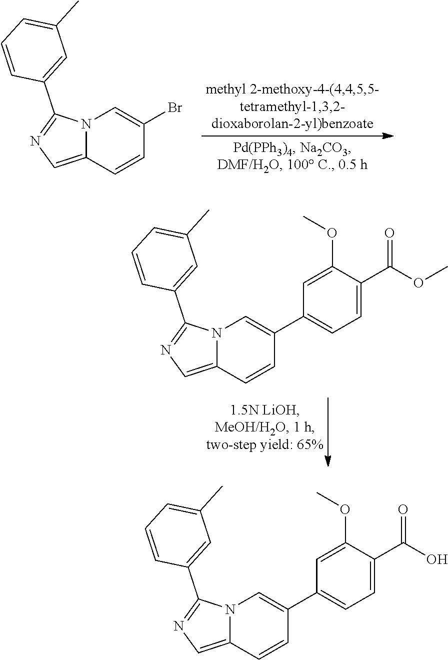 [SMILES: Cc1cccc(-c2ncc3ccc(Br)cn23)c1 ; COC(=O)c1ccc(-c2ccc3cnc(-c4cccc(C)c4)n3c2)cc1OC ; COc1cc(-c2ccc3cnc(-c4cccc(C)c4)n3c2)ccc1C(=O)O]

2-Methoxy-4-(3-(3-tolyl)imidazo[1,5-a]pyridin-6-yl)benzoic Acid: General procedures B1 was employed using 6-bromo-3-(3-tolyl)imidazo[1,5-a]pyridine (80 mg, 0.28 mmol, 1 eq.). The intermediate product was isolated by CombiFlash chromatography (silica gel, hexanes/ethyl acetate, 1:1) and hydrolyzed as described in General procedure C1, which led to a yellow solid (0.064 g, 65%). The product was carried to the next step without further characterization.

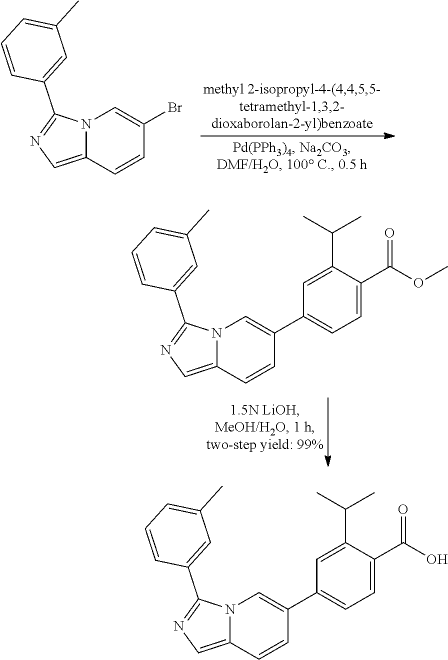 [SMILES: Cc1cccc(-c2ncc3ccc(Br)cn23)c1 ; COC(=O)c1ccc(-c2ccc3cnc(-c4cccc(C)c4)n3c2)cc1C(C)C ; Cc1cccc(-c2ncc3ccc(-c4ccc(C(=O)O)c(C(C)C)c4)cn23)c1]

2-Isopropyl-4-(3-(3-tolyl)imidazo[1,5-a]pyridin-6-yl)benzoic Acid: General procedure B1 was employed using 6-bromo-3-(3-tolyl)imidazo[1,5-a]pyridine (80 mg, 0.28 mmol, 1 eq.). The intermediate product was isolated by CombiFlash chromatography (silica gel, hexanes/ethyl acetate, 1:1) and hydrolyzed as described in General procedure C1, which led to a yellow solid (0.099 g, 99%) ¹H NMR (400 MHZ, DMSO-d₆) δ 8.54 (s, 1H), 8.02 (s, 1H), 7.93 (d, J=9.5 Hz, 1H), 7.79-7.70 (m, 4H), 7.64-7.52 (m, 3H), 7.51-7.41 (m, 2H), 3.46 (br, 1H), 2.43 (s, 3H), 1.24 (d, J=6.8 Hz, 6H).

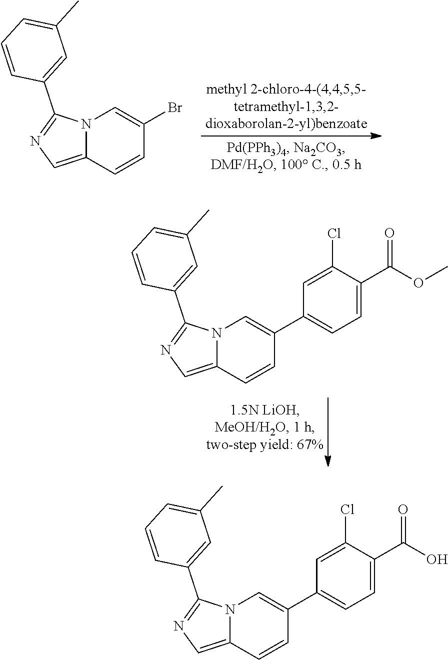 [SMILES: Cc1cccc(-c2ncc3ccc(Br)cn23)c1 ; COC(=O)c1ccc(-c2ccc3cnc(-c4cccc(C)c4)n3c2)cc1Cl ; Cc1cccc(-c2ncc3ccc(-c4ccc(C(=O)O)c(Cl)c4)cn23)c1]

2-Chloro-4-(3-(3-tolyl)imidazo[1,5-a]pyridin-6-yl)benzoic Acid: Methyl 2-chloro-4-(4,4,5,5-tetramethyl-1,3,2-dioxaborolan-2-yl)benzoate (80 mg, 0.27 mmol, 1 eq.), 6-bromo-3-(3-tolyl)imidazo[1,5-a]pyridine (93 mg, 0.32 mmol, 1.2 eq.), Na₂CO₃ (57 mg, 0.54 mmol, 2 eq.), and Pd(PPh₃)₄ (25 mg, 0.022 mmol, 0.08 eq.) were added to a microwave vial and sealed with a metal cap with septum. The vial was evacuated and backfilled with N₂ three times. Then DMF/H₂O (4:1, 5 mL) was degassed and injected into the reaction mixture. The suspension was stirred and heated at 100° C. for 0.5 h. After cooling to room temperature, the solution was diluted with ethyl acetate (15 mL) and washed with water (2×15 mL) and brine (1×15 mL). The organic layer was dried over MgSO₄, concentrated under reduced pressure, and purified by CombiFlash chromatography (silica gel, hexanes/ethyl acetate, 1:3). The intermediate product was then hydrolyzed as described in General procedure C1, which led to a yellow solid (66 mg, 67%). ¹H NMR (400 MHZ, DMSO-d₆) δ 8.56 (s, 1H), 7.86 (dd, J=5.0, 3.2 Hz, 2H), 7.79-7.65 (m, 4H), 7.59 (s, 1H), 7.46 (t, J=7.6 Hz, 1H), 7.30 (d, J=7.6 Hz, 1H), 7.22-7.17 (m, 1H), 2.40 (s, 3H).

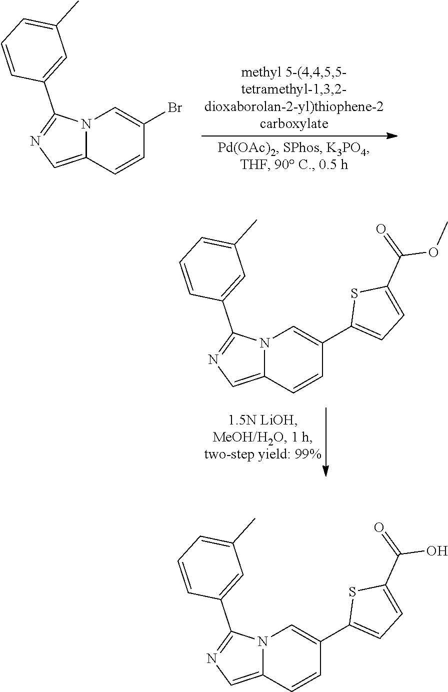 [SMILES: Cc1cccc(-c2ncc3ccc(Br)cn23)c1 ; COC(=O)c1ccc(-c2ccc3cnc(-c4cccc(C)c4)n3c2)s1 ; Cc1cccc(-c2ncc3ccc(-c4ccc(C(=O)O)s4)cn23)c1]

5-(3-(3-Tolyl)imidazo[1,5-a]pyridin-6-yl)thiophene-2-carboxylic Acid: General procedure B4 was employed using 6-bromo-3-(3-tolyl)imidazo[1,5-a]pyridine (100 mg, 0.35 mmol, 1 eq.). The intermediate product was isolated by CombiFlash chromatography (silica gel, hexanes/ethyl acetate, 1:1) and hydrolyzed as described in General procedure C4, which led to a yellow solid (115 mg, 99%). ¹H NMR (400 MHZ, DMSO-d₆) δ 8.58 (s, 1H), 7.75 (dd, J=9.4, 1.1 Hz, 1H), 7.71 (d, J=3.9 Hz, 1H), 7.68-7.64 (m, 2H), 7.58 (d, J=4.6 Hz, 2H), 7.48 (t, J=7.9 Hz, 1H), 7.32 (d, J=7.6 Hz, 1H), 7.17 (dd, J=9.4, 1.5 Hz, 1H), 2.41 (s, 3H).

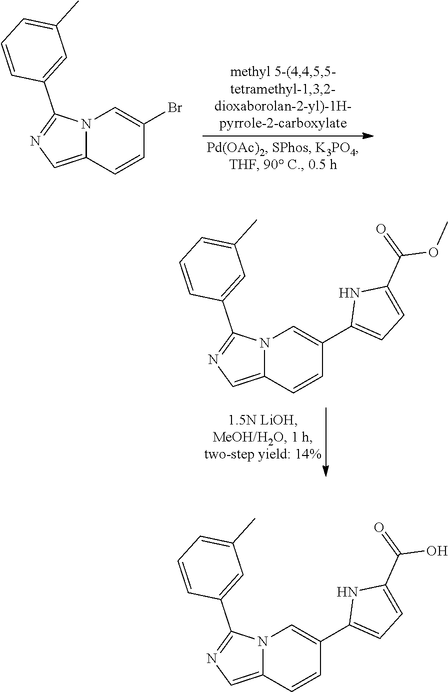 [SMILES: Cc1cccc(-c2ncc3ccc(Br)cn23)c1 ; COC(=O)c1ccc(-c2ccc3cnc(-c4cccc(C)c4)n3c2)[nH]1 ; Cc1cccc(-c2ncc3ccc(-c4ccc(C(=O)O)[nH]4)cn23)c1]

5-(3-(3-Tolyl)imidazo[1,5-a]pyridin-6-yl)-1H-pyrrole-2-carboxylic Acid: General procedure B4 was employed using 6-bromo-3-(3-tolyl)imidazo[1,5-a]pyridine (80 mg, 0.28 mmol, 1 eq.). The intermediate product was isolated by CombiFlash chromatography (silica gel, hexanes/ethyl acetate, 1:1) and hydrolyzed as described in General procedure C4, which led to a yellow solid (12 mg, 14%). The product was carried to the next step without further characterization.

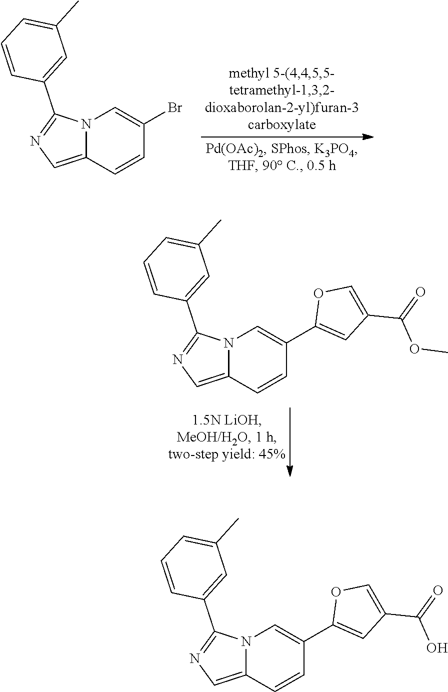 [SMILES: Cc1cccc(-c2ncc3ccc(Br)cn23)c1 ; COC(=O)c1coc(-c2ccc3cnc(-c4cccc(C)c4)n3c2)c1 ; Cc1cccc(-c2ncc3ccc(-c4cc(C(=O)O)co4)cn23)c1]

5-(3-(3-Tolyl)imidazo[1,5-a]pyridin-6-yl) furan-3-carboxylic Acid: General procedure B4 was employed using 6-bromo-3-(3-tolyl)imidazo[1,5-a]pyridine (80 mg, 0.28 mmol, 1 eq.). The intermediate product was isolated by CombiFlash chromatography (silica gel, hexanes/ethyl acetate, 1:1) and hydrolyzed as described in General procedure C4, which led to a yellow solid (40 mg, 45%). ¹H NMR (400 MHZ, DMSO-d₆) δ 8.55 (s, 1H), 8.33 (s, 1H), 7.73 (d, J=9.5 Hz, 1H), 7.63 (d, J=5.4 Hz, 2H), 7.57 (s, 1H), 7.48 (t, J=8.0 Hz, 1H), 7.33 (d, J=7.7 Hz, 1H), 7.29-7.19 (m, 2H), 2.41 (s, 3H).

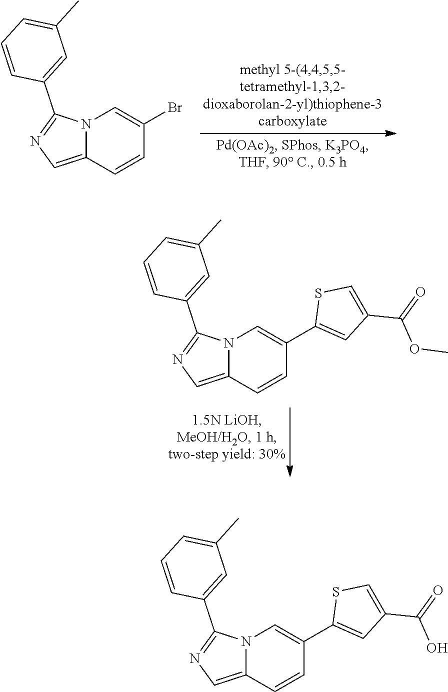 [SMILES: Cc1cccc(-c2ncc3ccc(Br)cn23)c1 ; COC(=O)c1csc(-c2ccc3cnc(-c4cccc(C)c4)n3c2)c1 ; Cc1cccc(-c2ncc3ccc(-c4cc(C(=O)O)cs4)cn23)c1]

5-(3-(3-Tolyl)imidazo[1,5-a]pyridin-6-yl)thiophene-3-carboxylic Acid: General procedure B4 was employed using 6-bromo-3-(3-tolyl)imidazo[1,5-a]pyridine (80 mg, 0.28 mmol, 1 eq.). The intermediate product was isolated by CombiFlash chromatography (silica gel, hexanes/ethyl acetate, 1:1) and hydrolyzed as described in General procedure C4, which led to a yellow solid (28 mg, 30%). ¹H NMR (400 MHZ, DMSO-d₆) δ 8.52 (s, 1H), 8.23 (s, 1H), 7.79 (s, 1H), 7.74 (d, J=9.6 Hz, 1H), 7.66 (d, J=6.6 Hz, 2H), 7.58 (s, 1H), 7.48 (t, J=7.7 Hz, 1H), 7.32 (d, J=7.7 Hz, 1H), 7.18 (d, J=9.4 Hz, 1H), 2.41 (s, 3H).

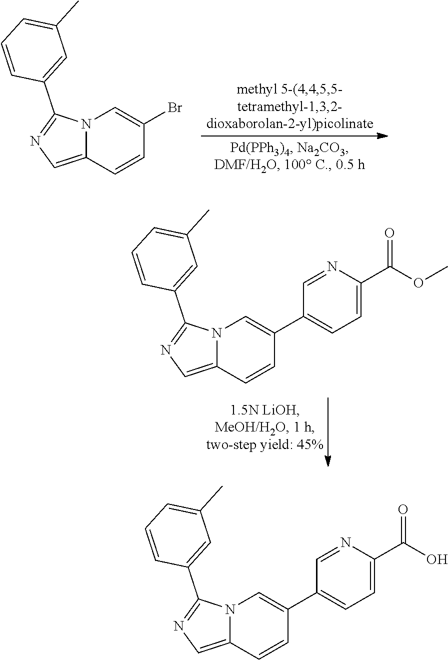 [SMILES: Cc1cccc(-c2ncc3ccc(Br)cn23)c1 ; COC(=O)c1ccc(-c2ccc3cnc(-c4cccc(C)c4)n3c2)cn1 ; Cc1cccc(-c2ncc3ccc(-c4ccc(C(=O)O)nc4)cn23)c1]

5-(3-(3-Tolyl)imidazo[1,5-a]pyridin-6-yl) picolinic Acid: General procedure B1 was employed using 6-bromo-3-(3-tolyl)imidazo[1,5-a]pyridine (20 mg, 0.070 mmol, 1 eq.). The intermediate product was isolated by CombiFlash chromatography (silica gel, ethyl acetate) and hydrolyzed as described in General procedure C1, which led to a yellow solid (10 mg, 44%). ¹H NMR (400 MHz, CDCl₃) δ 8.77 (s, 1H), 8.46 (q, J=1.3 Hz, 1H), 8.25 (d, J=8.1 Hz, 1H), 7.98 (dd, J=8.1, 2.3 Hz, 1H), 7.68-7.58 (m, 3H), 7.45 (t, J=7.6 Hz, 1H), 7.29 (d, J=7.5 Hz, 1H), 7.26-7.22 (m, 1H), 6.98 (dd, J=9.4, 1.4 Hz, 1H), 3.74-3.52 (m, 2H), 3.00-2.50 (m, 6H), 2.46 (s, 3H), 1.35-1.19 (m, 6H).

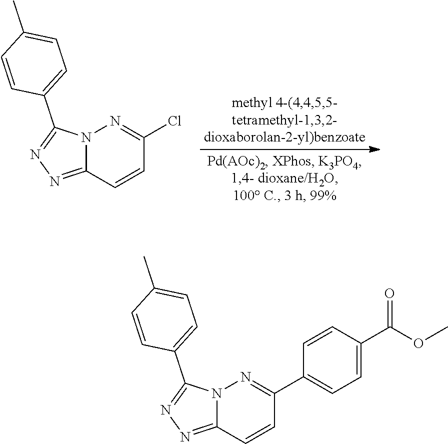 [SMILES: Cc1ccc(-c2nnc3ccc(Cl)nn23)cc1 ; COC(=O)c1ccc(-c2ccc3nnc(-c4ccc(C)cc4)n3n2)cc1]

Methyl 4-(3-(4-Tolyl)-[1,2,4]triazolo[4,3-b]pyridazin-6-yl)benzoate: General procedure B3 was employed using 6-chloro-3-(4-tolyl)-[1,2,4]triazolo[4,3-b]pyridazine (200 mg, 0.82 mmol, 1 eq.) and methyl 4-(4,4,5,5-tetramethyl-1,3,2-dioxaborolan-2-yl)benzoate (257 mg, 0.98 mmol, 1.2 eq.). A white solid (0.28 g, 99%) was isolated by CombiFlash chromatography (silica gel, hexanes/ethyl acetate, 1:4). ¹H NMR (400 MHZ, CDCl₃) δ 8.48 (d, J=8.3 Hz, 2H), 8.30 (d, J=9.7 Hz, 1H), 8.26 (d, J=8.7 Hz, 2H), 8.13 (d, J=8.5 Hz, 2H), 7.65 (d, J=9.7 Hz, 1H), 7.44 (d, J=8.0 Hz, 2H), 4.01 (s, 3H), 2.50 (s, 3H).

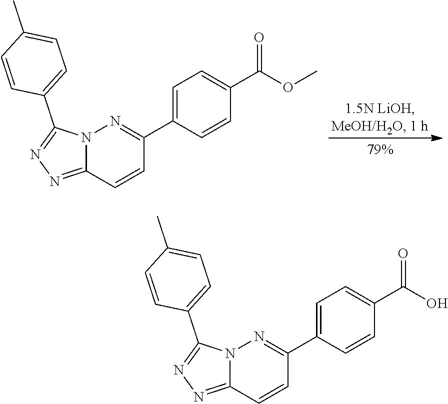 [SMILES: COC(=O)c1ccc(-c2ccc3nnc(-c4ccc(C)cc4)n3n2)cc1 ; Cc1ccc(-c2nnc3ccc(-c4ccc(C(=O)O)cc4)nn23)cc1]

4-(3-(4-Tolyl)-[1,2,4]triazolo[4,3-b]pyridazin-6-yl)benzoic Acid: General procedure C3 was employed using methyl 4-(3-(4-tolyl)-[1,2,4]triazolo[4,3-b]pyridazin-6-yl)benzoate (245 mg, 0.71 mmol, 1 eq.). A white solid (0.19 g, 79%) was isolated through filtration. ¹H NMR (400 MHZ, DMSO-d₆) δ 8.58 (d, J=9.8 Hz, 1H), 8.37 (d, J=8.3 Hz, 2H), 8.29 (d, J=8.5 Hz, 2H), 8.16 (d, J=8.5 Hz, 2H), 8.07 (d, J=9.8 Hz, 1H), 7.48 (d, J=8.0 Hz, 2H), 2.44 (s, 3H).

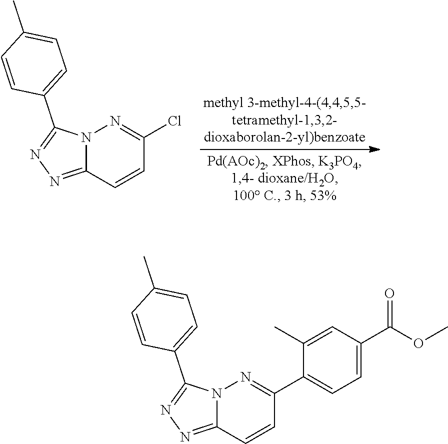 [SMILES: Cc1ccc(-c2nnc3ccc(Cl)nn23)cc1 ; COC(=O)c1ccc(-c2ccc3nnc(-c4ccc(C)cc4)n3n2)c(C)c1]

Methyl 3-Methyl-4-(3-(4-tolyl)-[1,2,4]triazolo[4,3-b]pyridazin-6-yl)benzoate: General procedure B3 was employed using 6-chloro-3-(4-tolyl)-[1,2,4]triazolo[4,3-b]pyridazine (300 mg, 1.2 mmol, 1 eq.) and methyl 3-methyl-4-(4,4,5,5-tetramethyl-1,3,2-dioxaborolan-2-yl)benzoate (406 mg, 1.5 mmol, 1.2 eq.). A white solid (0.23 g, 53%) was isolated by CombiFlash chromatography (silica gel, hexanes/ethyl acetate, 1:4). ¹H NMR (400 MHZ, CDCl₃) δ 8.42 (d, J=8.2 Hz, 2H), 8.27 (d, J=9.6 Hz, 1H), 8.09 (s, 1H), 8.05 (d, J=7.8 Hz, 1H), 7.59 (d, J=8.0 Hz, 1H), 7.37 (d, J=8.0 Hz, 2H), 7.31 (s, 1H), 4.00 (s, 3H), 2.54 (s, 3H), 2.46 (s, 3H).

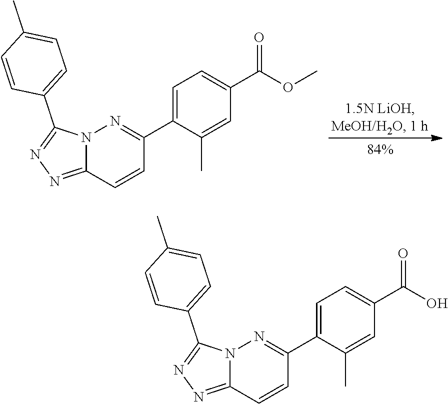 [SMILES: COC(=O)c1ccc(-c2ccc3nnc(-c4ccc(C)cc4)n3n2)c(C)c1 ; Cc1ccc(-c2nnc3ccc(-c4ccc(C(=O)O)cc4C)nn23)cc1]

3-Methyl-4-(3-(4-tolyl)-[1,2,4]triazolo[4,3-b]pyridazin-6-yl)benzoic Acid: General procedure C3 was employed using methyl 3-methyl-4-(3-(4-tolyl)-[1,2,4]triazolo[4,3-b]pyridazin-6-yl)benzoate (200 mg, 0.56 mmol, 1 eq.). A white solid (0.16 g, 84%) was isolated through filtration. ¹H NMR (400 MHZ, DMSO-d₆) δ 8.52 (d, J=9.6 Hz, 1H), 8.27 (d, J=8.1 Hz, 2H), 7.98 (s, 1H), 7.94 (d, J=8.2 Hz, 1H), 7.71 (d, J=8.0 Hz, 1H), 7.63 (d, J=9.6 Hz, 1H), 7.42 (d, J=8.1 Hz, 2H), 2.48 (s, 3H), 2.39 (s, 3H).

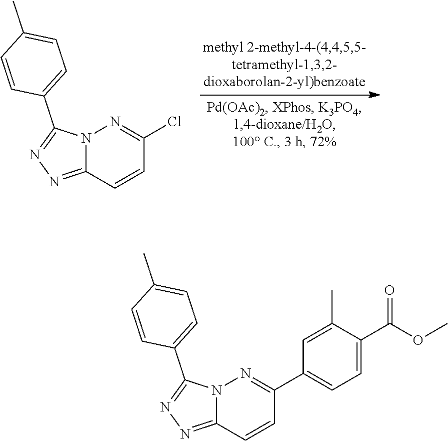 [SMILES: Cc1ccc(-c2nnc3ccc(Cl)nn23)cc1 ; COC(=O)c1ccc(-c2ccc3nnc(-c4ccc(C)cc4)n3n2)cc1C]

Methyl 2-Methyl-4-(3-(4-tolyl)-[1,2,4]triazolo[4,3-b]pyridazin-6-yl)benzoate: General procedure B3 was employed using 6-chloro-3-(4-tolyl)-[1,2,4]triazolo[4,3-b]pyridazine (300 mg, 1.2 mmol, 1 eq.) and methyl 2-methyl-4-(4,4,5,5-tetramethyl-1,3,2-dioxaborolan-2-yl)benzoate (406 mg, 1.5 mmol, 1.2 eq.). A white solid (0.32 g, 72%) was isolated by CombiFlash chromatography (silica gel, hexanes/ethyl acetate, 1:4). ¹H NMR (400 MHZ, CDCl₃) δ 8.48 (d, J=8.1 Hz, 2H), 8.28 (d, J=9.7 Hz, 1H), 8.13 (d, J=8.0 Hz, 1H), 7.95-7.88 (m, 2H), 7.63 (d, J=9.7 Hz, 1H), 7.44 (d, J=8.0 Hz, 2H), 3.98 (s, 3H), 2.76 (s, 3H), 2.50 (s, 3H).

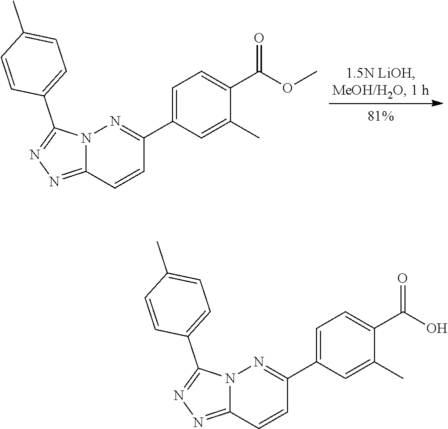 [SMILES: COC(=O)c1ccc(-c2ccc3nnc(-c4ccc(C)cc4)n3n2)cc1C ; Cc1ccc(-c2nnc3ccc(-c4ccc(C(=O)O)c(C)c4)nn23)cc1]

2-Methyl-4-(3-(4-tolyl)-[1,2,4]triazolo[4,3-b]pyridazin-6-yl)benzoic Acid: General procedure C3 was employed using methyl 2-methyl-4-(3-(4-tolyl)-[1,2,4]triazolo[4,3-b]pyridazin-6-yl)benzoate (300 mg, 0.84 mmol, 1 eq.). A white solid (0.23 g, 81%) was isolated through filtration. ¹H NMR (400 MHZ, DMSO-d₆) δ 8.54 (d, J=9.8 Hz, 1H), 8.36 (d, J=8.0 Hz, 2H), 8.07-8.01 (m, 3H), 7.94 (d, J=8.2 Hz, 1H), 7.48 (d, J=8.0 Hz, 2H), 2.63 (s, 3H), 2.43 (s, 3H).

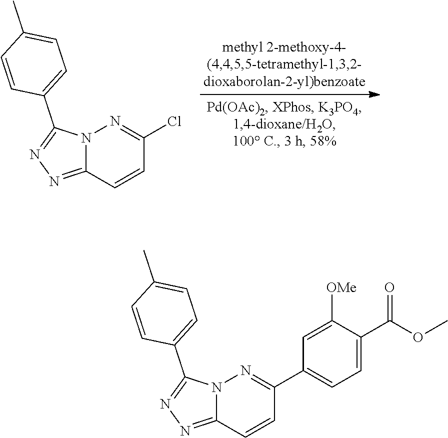 [SMILES: Cc1ccc(-c2nnc3ccc(Cl)nn23)cc1 ; COC(=O)c1ccc(-c2ccc3nnc(-c4ccc(C)cc4)n3n2)cc1OC]

Methyl 2-Methoxy-4-(3-(4-tolyl)-[1,2,4]triazolo[4,3-b]pyridazin-6-yl)benzoate: General procedure B3 was employed using 6-chloro-3-(4-tolyl)-[1,2,4]triazolo[4,3-b]pyridazine (300 mg, 1.2 mmol, 1 eq.) and methyl 2-methoxy-4-(4,4,5,5-tetramethyl-1,3,2-dioxaborolan-2-yl)benzoate (430 mg, 1.5 mmol, 1.2 eq.). A white solid (0.27 g, 58%) was isolated by CombiFlash chromatography (silica gel, hexanes/ethyl acetate, 1:4). ¹H NMR (400 MHZ, CDCl₃) δ 8.49 (d, J=8.2 Hz, 2H), 8.29 (d, J=9.7 Hz, 1H), 8.00 (d, J=8.0 Hz, 1H), 7.71 (d, J=1.6 Hz, 1H), 7.65-7.59 (m, 2H), 7.42 (d, J=8.0 Hz, 2H), 4.07 (s, 3H), 3.98 (s, 3H), 2.50 (s, 3H).

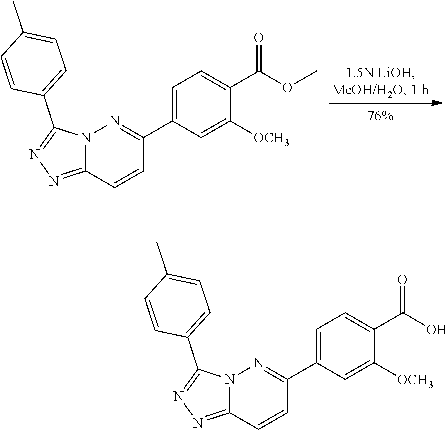 [SMILES: COC(=O)c1ccc(-c2ccc3nnc(-c4ccc(C)cc4)n3n2)cc1OC ; COc1cc(-c2ccc3nnc(-c4ccc(C)cc4)n3n2)ccc1C(=O)O]

2-Methoxy-4-(3-(4-tolyl)-[1,2,4]triazolo[4,3-b]pyridazin-6-yl)benzoic Acid: General procedure C3 was employed using methyl 2-methoxy-4-(3-(4-tolyl)-[1,2,4]triazolo[4,3-b]pyridazin-6-yl)benzoate (170 mg, 0.45 mmol, 1 eq.). A white solid (124 mg, 76%) was isolated through filtration. The product was carried to the next step without further characterization.

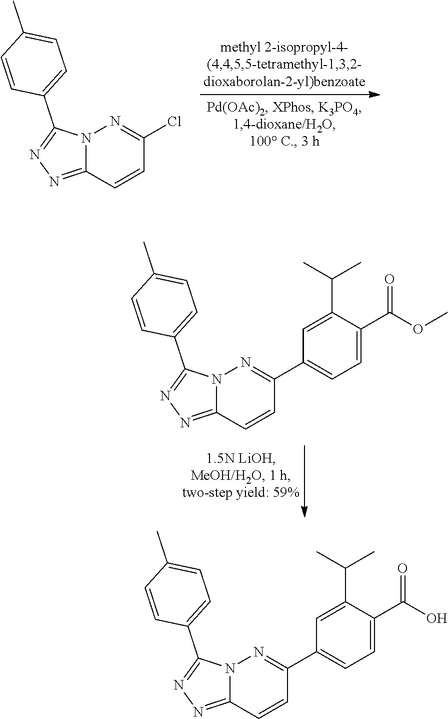 [SMILES: Cc1ccc(-c2nnc3ccc(Cl)nn23)cc1 ; COC(=O)c1ccc(-c2ccc3nnc(-c4ccc(C)cc4)n3n2)cc1C(C)C ; Cc1ccc(-c2nnc3ccc(-c4ccc(C(=O)O)c(C(C)C)c4)nn23)cc1]

General procedure B3 was employed using 6-chloro-3-(4-tolyl)-[1,2,4]triazolo[4,3-b]pyridazine (300 mg, 1.2 mmol, 1 eq.) and methyl 2-isopropyl-4-(4,4,5,5-tetramethyl-1,3,2-dioxaborolan-2-yl)benzoate (456 mg, 1.5 mmol, 1.2 eq.). The intermediate product was isolated by CombiFlash chromatography (silica gel, ethyl acetate) and hydrolyzed as described in General procedure C4, which led to a white solid (0.46 g, 59%). The product was carried to the next step without further characterization.

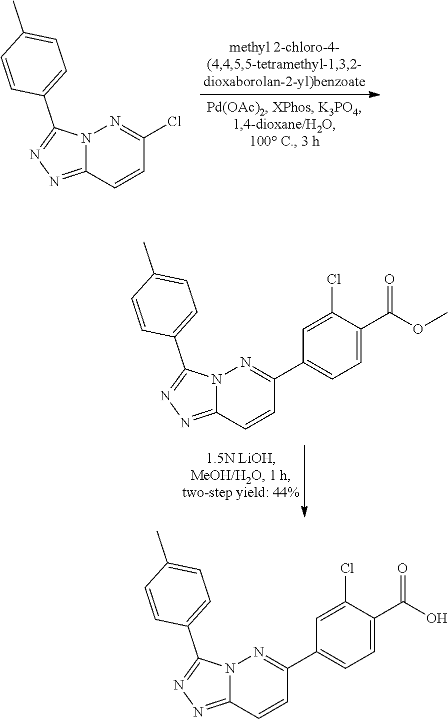 [SMILES: Cc1ccc(-c2nnc3ccc(Cl)nn23)cc1 ; COC(=O)c1ccc(-c2ccc3nnc(-c4ccc(C)cc4)n3n2)cc1Cl ; Cc1ccc(-c2nnc3ccc(-c4ccc(C(=O)O)c(Cl)c4)nn23)cc1]

2-Chloro-4-(3-(4-tolyl)-[1,2,4]triazolo[4,3-b]pyridazin-6-yl)benzoic Acid: Methyl 2-chloro-4-(4,4,5,5-tetramethyl-1,3,2-dioxaborolan-2-yl)benzoate (80 mg, 0.27 mmol, 1 eq.), 6-chloro-3-(4-tolyl)-[1,2,4]triazolo[4,3-b]pyridazine (78 mg, 0.32 mmol, 1.2 eq.), K₃PO₄ (115 mg, 0.54 mmol, 2 eq.), Pd(OAc)₂ (9.2 mg, 0.041 mmol, 0.15 eq.), and XPhos (58 mg, 0.12 mmol, 0.45 eq.) were added to a microwave vial and sealed with a metal cap with septum. The vial was evacuated and backfilled with N₂ three times. Then 1,4-dioxane/H₂O (5:1, 12 mL) was degassed and injected into the reaction mixture. The suspension was stirred and heated at 100° C. for 3-5 h. After cooling to room temperature, the solution was diluted with ethyl acetate (30 mL) and washed with water (2×30 mL) and brine (1×30 mL). The organic layer was dried over MgSO₄, concentrated under reduced pressure, and purified by CombiFlash chromatography (silica gel, hexanes/ethyl acetate, 1:4). The product was carried to hydrolysis using general procedure C3, leading to a dark yellow solid (52 mg, 44%). The product was carried to the next step without further characterization.

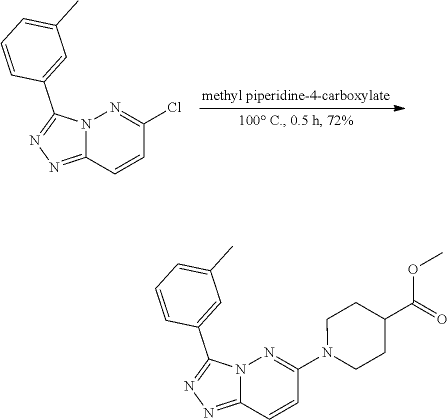 [SMILES: Cc1cccc(-c2nnc3ccc(Cl)nn23)c1 ; COC(=O)C1CCN(c2ccc3nnc(-c4cccc(C)c4)n3n2)CC1]

Methyl 1-(3-(3-Tolyl)-[1,2,4]triazolo[4,3-b]pyridazin-6-yl)piperidine-4-carboxylate: General procedure E1 was employed using 6-chloro-3-(3-tolyl)-[1,2,4]triazolo[4,3-b]pyridazine (241 mg, 0.99 mmol, 1 eq.) and methyl piperidine-4-carboxylate, HCl (885 mg, 4.9 mmol, 5 eq.). A white solid (0.25 g, 72%) was isolated by CombiFlash chromatography (silica gel, MeOH/CH₂Cl₂, 1:19). ¹H NMR (400 MHZ, CDCl₃) δ 8.34-8.27 (m, 2H), 7.93 (d, J=10.0 Hz, 1H), 7.42 (t, J=7.7 Hz, 1H), 7.29 (d, J=7.6 Hz, 1H), 6.97 (d, J=10.2 Hz, 1H), 4.12 (dt, J=13.8, 4.1 Hz, 2H), 3.73 (s, 3H), 3.24-3.14 (m, 2H), 2.69-2.60 (m, 1H), 2.46 (s, 3H), 2.09 (dd, J=13.8, 3.6 Hz, 2H), 1.94-1.82 (m, 2H).

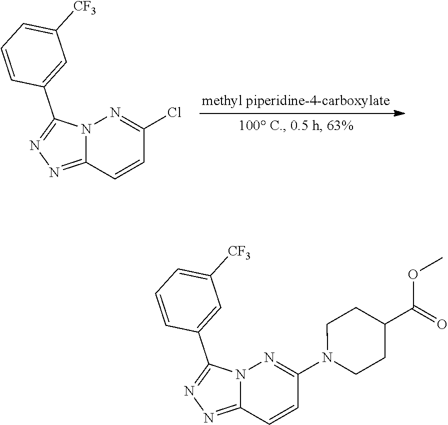 [SMILES: FC(F)(F)c1cccc(-c2nnc3ccc(Cl)nn23)c1 ; COC(=O)C1CCN(c2ccc3nnc(-c4cccc(C(F)(F)F)c4)n3n2)CC1]

Methyl 1-(3-(3-(Trifluoromethyl)phenyl)-[1,2,4]triazolo[4,3-b]pyridazin-6-yl)piperidine-4-carboxylate: General procedure E1 was employed using 6-chloro-3-(3-(trifluoromethyl)phenyl)-[1,2,4]triazolo[4,3-b]pyridazine (200 mg, 0.67 mmol, 1 eq.) and methyl piperidine-4-carboxylate, HCl (602 mg, 3.4 mmol, 5 eq.). A white solid (0.17 g, 63%) was isolated by CombiFlash chromatography (silica gel, MeOH/CH₂Cl₂, 1:19). ¹H NMR (400 MHZ, CDCl₃) δ 8.99 (s, 1H), 8.68 (d, J=7.9 Hz, 1H), 7.96 (d, J=10.2 Hz, 1H), 7.75-7.64 (m, 2H), 7.02 (d, J=10.2 Hz, 1H), 4.14 (dt, J=13.4, 3.9 Hz, 2H), 3.73 (s, 3H), 3.28-3.17 (m, 2H), 2.67 (tt, J=10.7, 4.0 Hz, 1H), 2.10 (dd, J=13.7, 3.9 Hz, 2H), 1.95-1.83 (m, 2H).

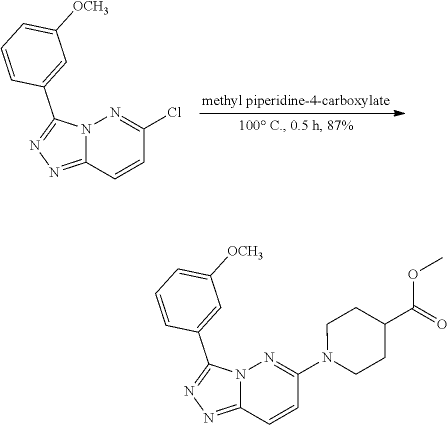 [SMILES: COc1cccc(-c2nnc3ccc(Cl)nn23)c1 ; COC(=O)C1CCN(c2ccc3nnc(-c4cccc(OC)c4)n3n2)CC1]

Methyl 1-(3-(3-Methoxyphenyl)-[1,2,4]triazolo[4,3-b]pyridazin-6-yl)piperidine-4-carboxylate: General procedure E1 was employed using 6-chloro-3-(3-methoxyphenyl)-[1,2,4]triazolo[4,3-b]pyridazine (100 mg, 0.38 mmol, 1 eq.) and methyl piperidine-4-carboxylate, HCl (345 mg, 1.9 mmol, 5 eq.). A white solid (0.12 g, 87%) was isolated by CombiFlash chromatography (silica gel, MeOH/CH₂Cl₂, 1:19). ¹H NMR (400 MHZ, CDCl₃) δ 8.14-8.08 (m, 2H), 7.93 (d, J=10.1 Hz, 1H), 7.44 (t, J=8.0 Hz, 1H), 7.03 (ddd, J=8.3, 2.6, 1.0 Hz, 1H), 6.98 (d, J=10.1 Hz, 1H), 4.13 (dt, J=13.5, 4.1 Hz, 2H), 3.91 (s, 3H), 3.73 (s, 3H), 3.24-3.14 (m, 2H), 2.65 (tt, J=10.7, 4.0 Hz, 1H), 2.09 (dd, J=13.7, 3.9 Hz, 2H), 1.95-1.81 (m, 2H).

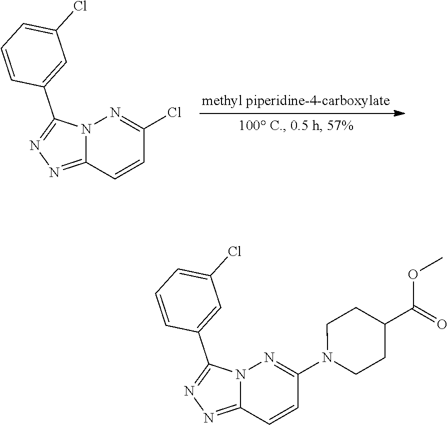 [SMILES: Clc1cccc(-c2nnc3ccc(Cl)nn23)c1 ; COC(=O)C1CCN(c2ccc3nnc(-c4cccc(Cl)c4)n3n2)CC1]

Methyl 1-(3-(3-Chlorophenyl)-[1,2,4]triazolo[4,3-b]pyridazin-6-yl)piperidine-4-carboxylate: General procedure E1 was employed using 6-chloro-3-(3-chlorophenyl)-[1,2,4]triazolo[4,3-b]pyridazine (100 mg, 0.38 mmol, 1 eq.) and methyl piperidine-4-carboxylate, HCl (345 mg, 1.9 mmol, 5 eq.). A white solid (0.080 g, 57%) was isolated by CombiFlash chromatography (silica gel, MeOH/CH₂Cl₂, 1:19). ¹H NMR (400 MHZ, CDCl₃) δ 8.60 (t, J=1.8 Hz, 1H), 8.40 (dt, J=7.2, 1.8 Hz, 1H), 7.94 (d, J=10.1 Hz, 1H), 7.49-7.42 (m, 2H), 7.00 (d, J=10.1 Hz, 1H), 4.13 (dt, J=13.5 Hz, 4.0 Hz, 2H), 3.73 (s, 3H), 3.27-3.15 (m, 2H), 2.66 (ddd, J=14.7, 10.6, 4.0 Hz, 1H), 2.11 (dd, J=13.7, 3.7 Hz, 2H), 1.96-1.81 (m, 2H).

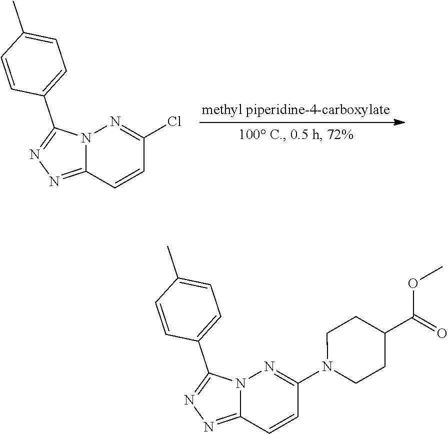 [SMILES: Cc1ccc(-c2nnc3ccc(Cl)nn23)cc1 ; COC(=O)C1CCN(c2ccc3nnc(-c4ccc(C)cc4)n3n2)CC1]

Methyl 1-(3-(4-Tolyl)-[1,2,4]triazolo[4,3-b]pyridazin-6-yl)piperidine-4-carboxylate: General procedure E2 was employed using 6-chloro-3-(4-tolyl)-[1,2,4]triazolo[4,3-b]pyridazine (200 mg, 0.82 mmol, 1 eq.) and methyl piperidine-4-carboxylate. A white solid (0.21 g, 72%) was isolated through filtration. ¹H NMR (400 MHZ, CDCl₃) δ 8.39 (d, J=8.2 Hz, 2H), 7.94 (d, J=10.1 Hz, 1H), 7.37 (d, J=8.0 Hz, 2H), 6.98 (d, J=10.1 Hz, 1H), 4.13 (dt, J=13.4, 4.0 Hz, 2H), 3.75 (s, 3H), 3.25-3.16 (m, 2H), 2.66 (td, J=10.7, 5.2 Hz, 1H), 2.46 (s, 3H), 2.11 (dd, J=13.5, 3.8 Hz, 2H), 1.96-1.85 (m, 2H).

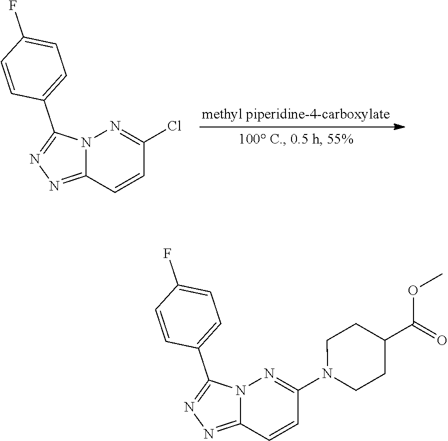 [SMILES: Fc1ccc(-c2nnc3ccc(Cl)nn23)cc1 ; COC(=O)C1CCN(c2ccc3nnc(-c4ccc(F)cc4)n3n2)CC1]

Methyl 1-(3-(4-Fluorophenyl)-[1,2,4]triazolo[4,3-b]pyridazin-6-yl)piperidine-4-carboxylate: General procedure E1 was employed using 6-chloro-3-(4-fluorophenyl)-[1,2,4]triazolo[4,3-b]pyridazine (150 mg, 0.60 mmol, 1 eq.) and methyl piperidine-4-carboxylate, HCl (542 mg, 3.0 mmol, 5 eq.). A white solid (0.12 g, 55%) was isolated by CombiFlash chromatography (silica gel, MeOH/CH₂Cl₂, 1:19). ¹H NMR (400 MHZ, CDCl₃) δ 8.49 (td, J=6.1 Hz, 2H), 7.93 (d, J=10.1 Hz, 1H), 7.23 (t, J=8.7 Hz, 2H), 6.99 (d, J=10.1 Hz, 1H), 4.11 (dt, J=13.9, 4.1 Hz, 2H), 3.73 (s, 3H), 3.24-3.15 (m, 2H), 2.65 (tt, J=10.7, 4.0 Hz, 1H), 2.10 (dd, J=13.8, 3.8 Hz, 2H), 1.94-1.83 (m, 2H).

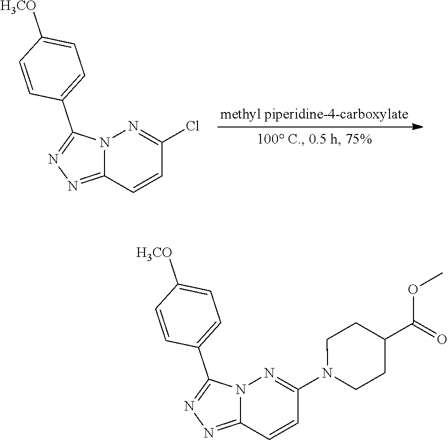 [SMILES: COc1ccc(-c2nnc3ccc(Cl)nn23)cc1 ; COC(=O)C1CCN(c2ccc3nnc(-c4ccc(OC)cc4)n3n2)CC1]

Methyl 1-(3-(4-Methoxyphenyl)-[1,2,4]triazolo[4,3-b]pyridazin-6-yl)piperidine-4-carboxylate: General procedure E1 was employed using 6-chloro-3-(4-methoxyphenyl)-[1,2,4]triazolo[4,3-b]pyridazine (100 mg, 0.38 mmol, 1 eq.) and methyl piperidine-4-carboxylate, HCl (345 mg, 1.9 mmol, 5 eq.). A white solid (0.11 g, 75%) was isolated by CombiFlash chromatography (silica gel, MeOH/CH₂Cl₂, 1:19). ¹H NMR (400 MHZ, CDCl₃) δ 8.43 (d, J=6.9 Hz, 2H), 7.91 (d, J=10.1 Hz, 1H), 7.06 (d, J=9.1 Hz, 2H), 6.95 (d, J=10.1 Hz, 1H), 4.10 (dt, J=13.5, 4.1 Hz, 2H), 3.90 (s, 3H), 3.73 (s, 3H), 3.22-3.13 (m, 2H), 2.64 (tt, J=10.8, 4.1 Hz, 1H), 2.09 (dd, J=13.6, 3.8 Hz, 2H), 1.94-1.83 (m, 2H).

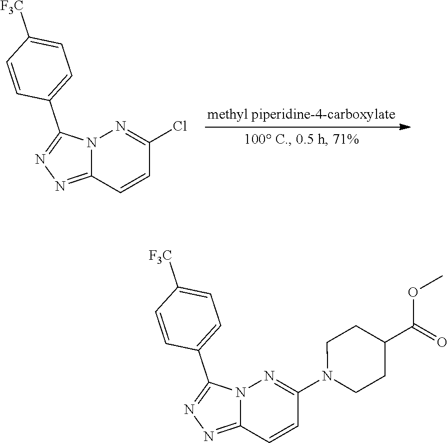 [SMILES: FC(F)(F)c1ccc(-c2nnc3ccc(Cl)nn23)cc1 ; COC(=O)C1CCN(c2ccc3nnc(-c4ccc(C(F)(F)F)cc4)n3n2)CC1]

Methyl 1-(3-(4-(Trifluoromethyl)phenyl)-[1,2,4]triazolo[4,3-b]pyridazin-6-yl)piperidine-4-carboxylate: General procedure E1 was employed using 6-chloro-3-(4-(trifluoromethyl)-phenyl)-[1,2,4]triazolo[4,3-b]pyridazine (100 mg, 0.34 mmol, 1 eq.) and methyl piperidine-4-carboxylate, HCl (301 mg, 1.7 mmol, 5 eq.). A white solid (0.10 g, 71%) was isolated by CombiFlash chromatography (silica gel, MeOH/CH₂Cl₂, 1:19). ¹H NMR (400 MHZ, CDCl₃) δ 8.65 (d, J=8.3 Hz, 2H), 7.96 (d, J=10.1 Hz, 1H), 7.80 (d, J=8.3 Hz, 2H), 7.03 (d, J=10.0 Hz, 1H), 4.12 (dt, J=14.0, 4.0 Hz, 2H), 3.27-3.16 (m, 2H), 2.67 (tt, J=10.7, 4.0 Hz, 1H), 2.11 (dd, J=13.7, 3.9 Hz, 2H), 1.95-1.82 (m, 2H).

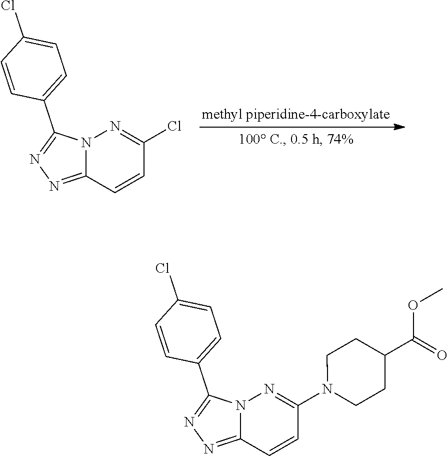 [SMILES: Clc1ccc(-c2nnc3ccc(Cl)nn23)cc1 ; COC(=O)C1CCN(c2ccc3nnc(-c4ccc(Cl)cc4)n3n2)CC1]

Methyl 1-(3-(4-Chlorophenyl)-[1,2,4]triazolo[4,3-b]pyridazin-6-yl)piperidine-4-carboxylate: General procedure E1 was employed using 6-chloro-3-(4-chlorophenyl)-[1,2,4]triazolo[4,3-b]-pyridazine (100 mg, 0.38 mmol, 1 eq.) and methyl piperidine-4-carboxylate, HCl (339 mg, 1.9 mmol, 5 eq.). A white solid (0.10 g, 74%) was isolated by CombiFlash chromatography (silica gel, MeOH/CH₂Cl₂, 1:19). ¹H NMR (400 MHZ, CDCl₃) δ 8.45 (d, J=8.7 Hz, 2H), 7.93 (d, J=10.1 Hz, 1H), 7.51 (d, J=8.7 Hz, 2H), 6.99 (d, J=10.1 Hz, 1H), 4.10 (dt, J=13.8, 3.8 Hz, 2H), 3.73 (s, 3H), 3.23-3.14 (m, 2H), 2.65 (tt, J=10.7, 4.0 Hz, 1H), 2.10 (dd, J=13.7, 3.7 Hz, 2H), 1.94-1.82 (m, 2H).

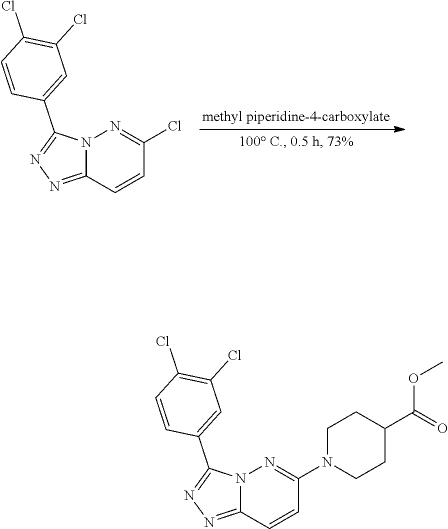 [SMILES: Clc1ccc2nnc(-c3ccc(Cl)c(Cl)c3)n2n1 ; COC(=O)C1CCN(c2ccc3nnc(-c4ccc(Cl)c(Cl)c4)n3n2)CC1]

Methyl 1-(3-(3,4-Dichlorophenyl)-[1,2,4]triazolo[4,3-b]pyridazin-6-yl)piperidine-4-carboxylate: General procedure E1 was employed using 6-chloro-3-(3,4-dichlorophenyl)-[1,2,4]triazolo[4,3-b]pyridazine (245 mg, 0.82 mmol, 1 eq.) and methyl piperidine-4-carboxylate, HCl (734 mg, 4.1 mmol, 5 eq.). A white solid (0.24 g, 73%) was isolated by CombiFlash chromatography (silica gel, MeOH/CH₂Cl₂, 1:19). ¹H NMR (400 MHZ, CDCl₃) δ 8.74 (d, J=2.0 Hz, 1H), 8.37 (dd, J=8.5, 2.0 Hz, 1H), 7.95 (d, J=10.2 Hz, 1H), 7.61 (d, J=8.5 Hz, 1H), 7.01 (d, J=10.2 Hz, 1H), 4.13 (dt, J=13.7, 4.2 Hz, 2H), 3.28-3.17 (m, 2H), 2.67 (tt, J=10.7, 4.0 Hz, 1H), 2.11 (dd, J=13.7, 3.8 Hz, 2H), 1.96-1.83 (m, 2H).

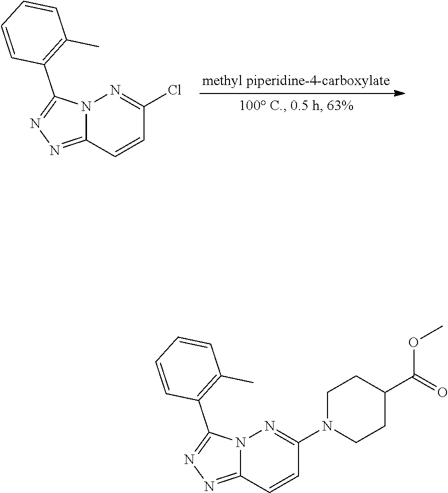 [SMILES: Cc1ccccc1-c1nnc2ccc(Cl)nn12 ; COC(=O)C1CCN(c2ccc3nnc(-c4ccccc4C)n3n2)CC1]

Methyl 1-(3-(2-Tolyl)-[1,2,4]triazolo[4,3-b]pyridazin-6-yl)piperidine-4-carboxylate: General procedure E1 was employed using 6-chloro-3-(2-tolyl)-[1,2,4]triazolo[4,3-b]pyridazine (200 mg, 0.82 mmol, 1 eq.) and methyl piperidine-4-carboxylate, HCl (734 mg, 4.1 mmol, 5 eq.). A white solid (0.18 g, 63%) was isolated by CombiFlash chromatography (silica gel, MeOH/CH₂Cl₂, 1:19). ¹H NMR (400 MHZ, CDCl₃) δ 7.92 (d, J=10.1 Hz, 1H), 7.71 (dd, J=7.7, 1.4 Hz, 1H), 7.46-7.28 (m, 3H), 6.98 (d, J=10.2 Hz, 1H), 4.02 (dt, J=13.6, 4.1 Hz, 2H), 3.70 (s, 3H), 3.10-3.01 (m, 2H), 2.57 (tt, J=10.7, 4.0 Hz, 1H), 2.43 (s, 3H), 2.01 (dd, J=13.7, 3.9 Hz, 2H), 1.86-1.72 (m, 2H).

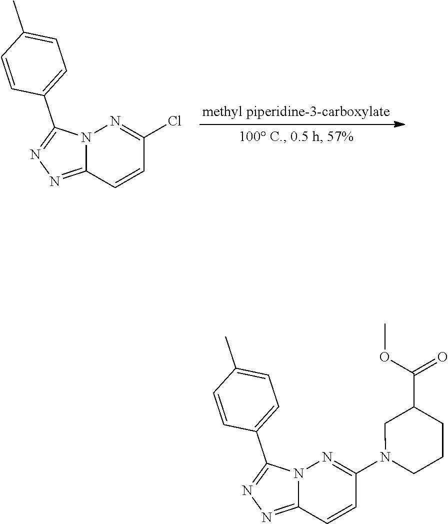 [SMILES: Cc1ccc(-c2nnc3ccc(Cl)nn23)cc1 ; COC(=O)C1CCCN(c2ccc3nnc(-c4ccc(C)cc4)n3n2)C1]

Methyl 1-(3-(4-Tolyl)-[1,2,4]triazolo[4,3-b]pyridazin-6-yl)piperidine-3-carboxylate: General procedure E2 was employed using 6-chloro-3-(4-tolyl)-[1,2,4]triazolo[4,3-b]pyridazine (200 mg, 0.82 mmol, 1 eq.) and methyl piperidine-3-carboxylate. A white solid (0.17 g, 57%) was isolated through filtration. ¹H NMR (400 MHz, CDCl₃) δ 8.40 (d, J=8.3 Hz, 2H), 7.92 (d, J=10.1 Hz, 1H), 7.34 (d, J=8.0 Hz, 2H), 7.00 (d, J=10.1 Hz, 1H), 4.16 (dd, J=13.4, 3.8 Hz, 1H), 3.87 (dt, J=13.3, 4.4 Hz, 1H), 3.75 (s, 3H), 3.44 (dd, J=13.3, 9.3 Hz, 1H), 3.39-3.27 (m, 1H), 2.76 (tt, J=9.0, 4.0 Hz, 1H), 2.44 (s, 3H), 2.15-2.06 (m, 1H), 1.97-1.81 (m, 2H), 1.75-1.64 (m, 1H).

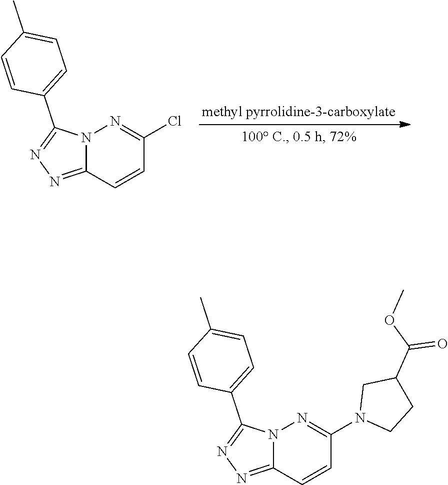 [SMILES: Cc1ccc(-c2nnc3ccc(Cl)nn23)cc1 ; COC(=O)C1CCN(c2ccc3nnc(-c4ccc(C)cc4)n3n2)C1]

Methyl 1-(3-(4-Tolyl)-[1,2,4]triazolo[4,3-b]pyridazin-6-yl)pyrrolidine-3-carboxylate: General procedure E1 was employed using 6-chloro-3-(4-tolyl)-[1,2,4]triazolo[4,3-b]pyridazine (200 mg, 0.82 mmol, 1 eq.) and methyl pyrrolidine-3-carboxylate, HCl (677 mg, 4.1 mmol, 5 eq.). A white solid (0.20 g, 72%) was isolated by CombiFlash chromatography (silica gel, MeOH/CH₂Cl₂, 1:19). ¹H NMR (400 MHZ, CDCl₃) δ 8.46 (d, J=8.3 Hz, 2H), 7.93 (d, J=9.9 Hz, 1H), 7.36 (d, J=8.0 Hz, 2H), 6.75 (d, J=10.0 Hz, 1H), 3.87 (d, J=6.5 Hz, 2H), 3.79 (s, 3H), 3.79-3.61 (m, 2H), 3.32 (p, J=7.1 Hz, 1H), 2.46 (s, 3H), 2.40 (qd, J=7.1, 2.0 Hz, 2H).

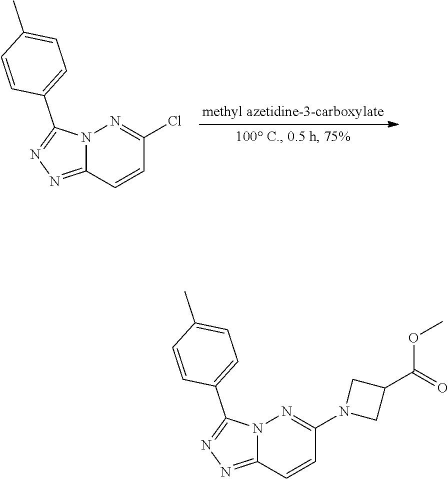 [SMILES: Cc1ccc(-c2nnc3ccc(Cl)nn23)cc1 ; COC(=O)C1CN(c2ccc3nnc(-c4ccc(C)cc4)n3n2)C1]

Methyl 1-(3-(4-Tolyl)-[1,2,4]triazolo[4,3-b]pyridazin-6-yl)azetidine-3-carboxylate: General procedure E1 was employed using 6-chloro-3-(4-tolyl)-[1,2,4]triazolo[4,3-b]pyridazine (200 mg, 0.82 mmol, 1 eq.) and methyl azetidine-3-carboxylate, HCl (620 mg, 4.1 mmol, 5 eq.). A white solid (0.20 g, 75%) was isolated by CombiFlash chromatography (silica gel, MeOH/CH₂Cl₂, 1:19). ¹H NMR (400 MHZ, CDCl₃) δ 8.39 (d, J=8.3 Hz, 2H), 7.92 (d, J=9.8 Hz, 1H), 7.33 (d, J=8.1 Hz, 2H), 6.51 (d, J=9.8 Hz, 1H), 4.40-4.33 (m, 4H), 3.80 (s, 3H), 3.67 (p, J=7.8 Hz, 1H), 2.43 (s, 3H).

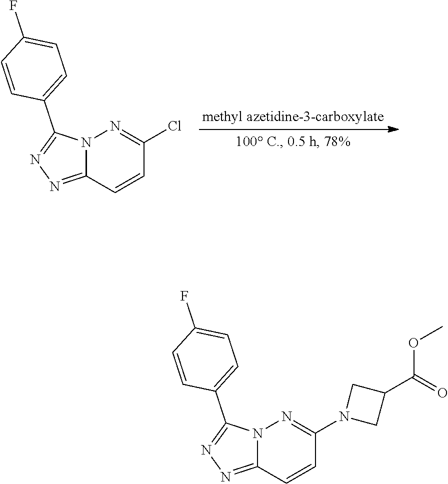 [SMILES: Fc1ccc(-c2nnc3ccc(Cl)nn23)cc1 ; COC(=O)C1CN(c2ccc3nnc(-c4ccc(F)cc4)n3n2)C1]

Methyl 1-(3-(4-Fluorophenyl)-[1,2,4]triazolo[4,3-b]pyridazin-6-yl)azetidine-3-carboxylate: General procedure E1 was employed using 6-chloro-3-(4-fluorophenyl)-[1,2,4]triazolo[4,3-b]pyridazine (150 mg, 0.60 mmol, 1 eq.) and methyl azetidine-3-carboxylate, HCl (457 mg, 3.0 mmol, 5 eq.). A white solid (0.15 g, 78%) was isolated by CombiFlash chromatography (silica gel, MeOH/CH₂Cl₂, 1:19). ¹H NMR (400 MHZ, CDCl₃) δ 8.56-8.47 (m, 2H), 7.93 (d, J=9.8 Hz, 1H), 7.24-7.16 (m, 2H), 6.53 (d, J=9.8 Hz, 1H), 4.40-4.35 (m, 4H), 3.80 (s, 3H), 3.69-3.63 (m, 1H).

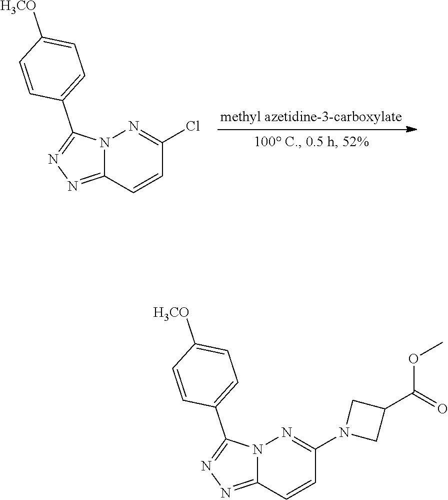 [SMILES: COc1ccc(-c2nnc3ccc(Cl)nn23)cc1 ; COC(=O)C1CN(c2ccc3nnc(-c4ccc(OC)cc4)n3n2)C1]

Methyl 1-(3-(4-Methoxyphenyl)-[1,2,4]triazolo[4,3-b]pyridazin-6-yl)azetidine-3-carboxylate: General procedure E1 was employed using 6-chloro-3-(4-methoxyphenyl)-[1,2,4]triazolo[4,3-b]pyridazine (150 mg, 0.58 mmol, 1 eq.) and methyl azetidine-3-carboxylate, HCl (436 mg, 2.9 mmol, 5 eq.). A white solid (0.10 g, 52%) was isolated by CombiFlash chromatography (silica gel, MeOH/CH₂Cl₂, 1:19). ¹H NMR (400 MHZ, CDCl₃) δ 8.46 (d, J=7.7 Hz, 2H), 7.91 (dt, J=9.6, 2.0 Hz, 1H), 7.04 (d, J=7.3 Hz, 2H), 6.50 (dd, J=9.9, 2.0 Hz, 1H), 4.42-4.32 (m, 4H), 3.89 (s, 3H), 3.80 (s, 3H), 3.69-3.63 (m, 1H).

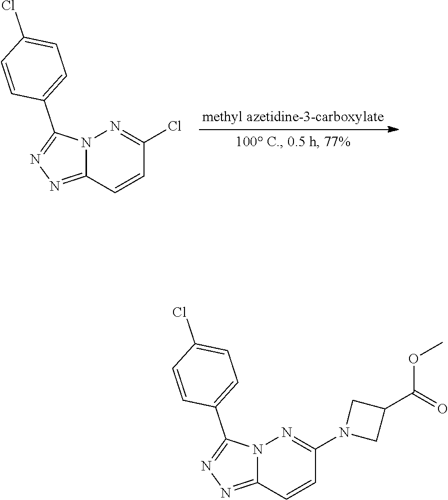 [SMILES: Clc1ccc(-c2nnc3ccc(Cl)nn23)cc1 ; COC(=O)C1CN(c2ccc3nnc(-c4ccc(Cl)cc4)n3n2)C1]

Methyl 1-(3-(4-Chlorophenyl)-[1,2,4]triazolo[4,3-b]pyridazin-6-yl)azetidine-3-carboxylate: General procedure E1 was employed using 6-chloro-3-(4-cholorophenyl)-[1,2,4]triazolo[4,3-b]pyridazine (153 mg, 0.58 mmol, 1 eq.) and methyl azetidine-3-carboxylate, HCl (436 mg, 2.9 mmol, 5 eq.). A white solid (0.15 g, 77%) was isolated by CombiFlash chromatography (silica gel, MeOH/CH₂Cl₂, 1:19). ¹H NMR (400 MHZ, CDCl₃) δ 8.51-8.45 (dt, J=8.7, 2.7 Hz, 2H), 7.94 (d, J=9.8 Hz, 1H), 7.52-7.45 (dt, J=8.6, 2.3 Hz, 2H), 6.54 (d, J=9.8 Hz, 1H), 4.41-4.35 (m, 4H), 3.81 (s, 3H), 3.72-3.64 (p, J=7.9 Hz, 1H).

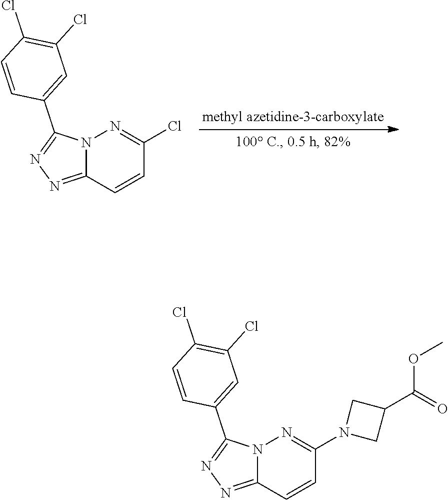 [SMILES: Clc1ccc2nnc(-c3ccc(Cl)c(Cl)c3)n2n1 ; COC(=O)C1CN(c2ccc3nnc(-c4ccc(Cl)c(Cl)c4)n3n2)C1]

Methyl 1-(3-(3,4-Dichlorophenyl)-[1,2,4]triazolo[4,3-b]pyridazin-6-yl)azetidine-3-carboxylate: General procedure E1 was employed using 6-chloro-3-(3,4-dicholorophenyl)-[1,2,4]triazolo[4,3-b]pyridazine (245 mg, 0.82 mmol, 1 eq.) and methyl azetidine-3-carboxylate, HCl (372 mg, 2.5 mmol, 3 eq.). A white solid (0.26 g, 82%) was isolated by CombiFlash chromatography (silica gel, MeOH/CH₂Cl₂, 1:19). The product was used for the next step without further characterization.

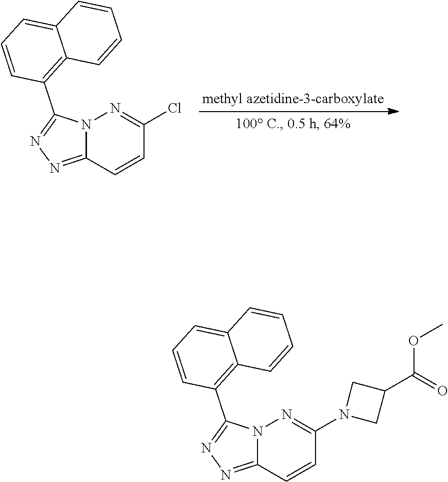 [SMILES: Clc1ccc2nnc(-c3cccc4ccccc34)n2n1 ; COC(=O)C1CN(c2ccc3nnc(-c4cccc5ccccc45)n3n2)C1]

Methyl 1-(3-(Naphthalen-1-yl)-[1,2,4]triazolo[4,3-b]pyridazin-6-yl)azetidine-3-carboxylate: General procedure E1 was employed using 5-chloro-3-(1-naphthyl)-1,2,3a,4-tetraazaindene (200 mg, 0.71 mmol, 1 eq.) and methyl azetidine-3-carboxylate, HCl (324 mg, 2.1 mmol, 3 eq.). A white solid (0.16 g, 64%) was isolated by CombiFlash chromatography (silica gel, MeOH/CH₂Cl₂, 1:19). The product was used for the next step without further characterization.

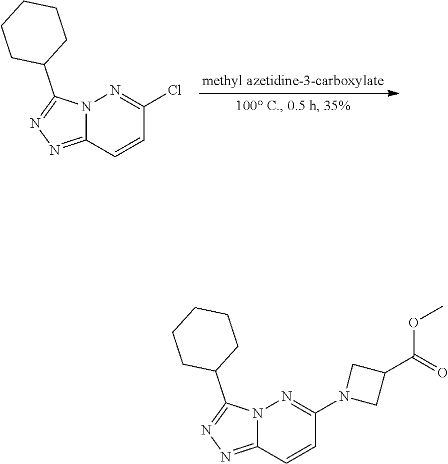 [SMILES: Clc1ccc2nnc(C3CCCCC3)n2n1 ; COC(=O)C1CN(c2ccc3nnc(C4CCCCC4)n3n2)C1]

Methyl 1-(3-Cyclohexyl-[1,2,4]triazolo[4,3-b]pyridazin-6-yl)azetidine-3-carboxylate: General procedure E1 was employed using 5-chloro-3-cyclohexyl-1,2,3a,4-tetraazaindene (200 mg, 0.85 mmol, 1 eq.) and methyl azetidine-3-carboxylate, HCl (384 mg, 2.5 mmol, 3 eq.). A white solid (0.09 g, 35%) was isolated by CombiFlash chromatography (silica gel, MeOH/CH₂Cl₂, 1:19). The product was used for the next step without further characterization.

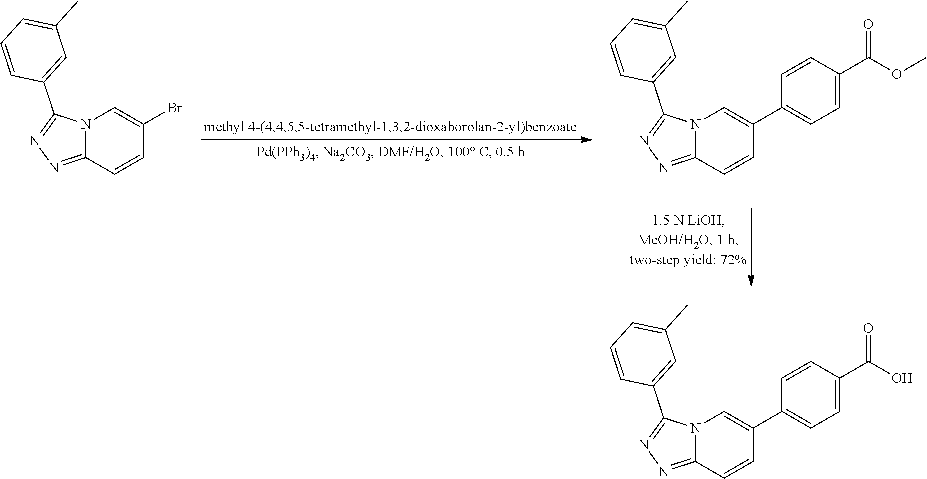 [SMILES: Cc1cccc(-c2nnc3ccc(Br)cn23)c1 ; COC(=O)c1ccc(-c2ccc3nnc(-c4cccc(C)c4)n3c2)cc1 ; Cc1cccc(-c2nnc3ccc(-c4ccc(C(=O)O)cc4)cn23)c1]

4-(3-(3-Tolyl)-[1,2,4]triazolo[4,3-a]pyridin-6-yl)benzoic Acid: General procedure B2 was employed using methyl 4-(3-(3-tolyl)-[1,2,4]triazolo[4,3-a]pyridin-6-yl)benzoate (260 mg, 0.76 mmol, 1 eq.). The intermediate product was isolated by CombiFlash chromatography (silica gel, hexanes/ethyl acetate, 1:3) and hydrolyzed as described in General procedure C2, which led to a white solid (0.18 g, 72%). ¹H NMR (400 MHZ, MeOD) δ 8.65 (t, J=1.4 Hz, 1H), 8.17 (d, J=8.4 Hz, 2H), 7.92 (qd, J=9.7, 1.4 Hz, 2H), 7.83 (d, J=8.4 Hz, 2H), 7.79-7.72 (m, 2H), 7.58 (t, J=7.6 Hz, 1H), 7.49 (d, J=7.6 Hz, 1H), 2.52 (s, 3H).

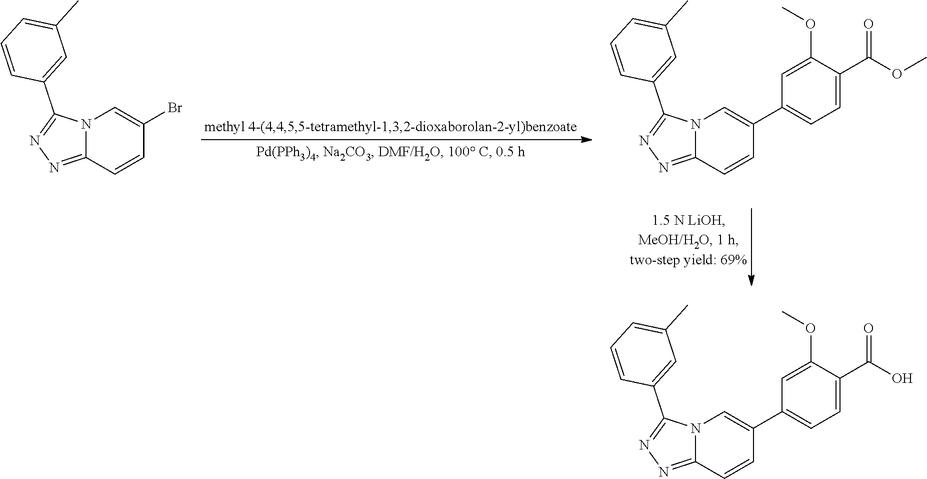 [SMILES: Cc1cccc(-c2nnc3ccc(Br)cn23)c1 ; COC(=O)c1ccc(-c2ccc3nnc(-c4cccc(C)c4)n3c2)cc1OC ; COc1cc(-c2ccc3nnc(-c4cccc(C)c4)n3c2)ccc1C(=O)O]

2-Methoxy-4-(3-(3-tolyl)-[1,2,4]triazolo[4,3-a]pyridin-6-yl)benzoic Acid: General procedure B2 was employed using methyl 6-bromo-3-(3-tolyl)-[1,2,4]triazolo[4,3-a]pyridine (170 mg, 0.59 mmol, 1 eq.). The intermediate product was isolated by CombiFlash chromatography (silica gel, hexanes/ethyl acetate, 1:3) and hydrolyzed as described in General procedure C2, which led to a white solid (0.15 g, 69%). The product was carried to the next step without further characterization.

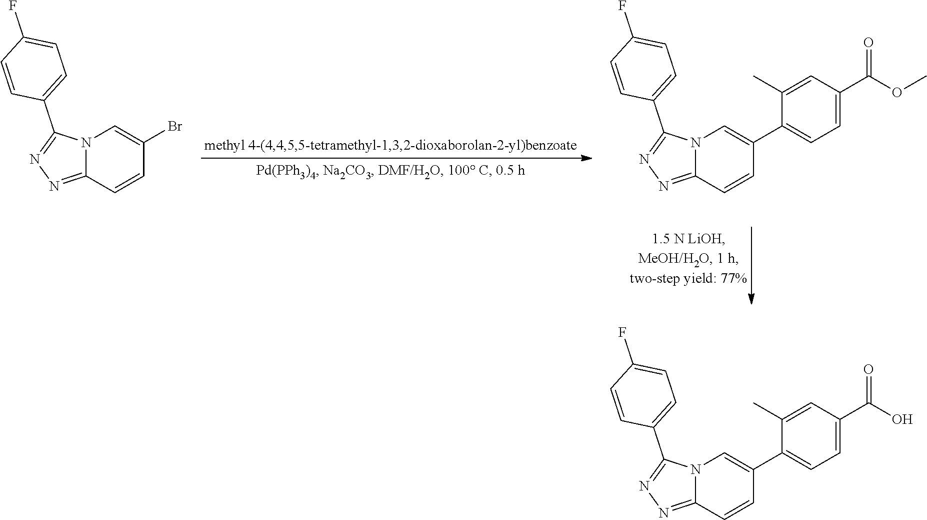 [SMILES: Fc1ccc(-c2nnc3ccc(Br)cn23)cc1 ; COC(=O)c1ccc(-c2ccc3nnc(-c4ccc(F)cc4)n3c2)c(C)c1 ; Cc1cc(C(=O)O)ccc1-c1ccc2nnc(-c3ccc(F)cc3)n2c1]

4-(3-(4-Fluorophenyl)-[1,2,4]triazolo[4,3-a]pyridin-6-yl)-3-methylbenzoic Acid: General procedures B2 was employed using methyl 4-(3-(4-fluorophenyl)-[1,2,4]triazolo[4,3-a]pyridin-6-yl)-3-methylbenzoate (270 mg, 0.75 mmol, 1 eq.). The intermediate product was isolated by CombiFlash chromatography (silica gel, hexanes/ethyl acetate, 1:3) and hydrolyzed as described in General procedure C2, which led to a white solid (0.20 g, 77%). ¹H NMR (400 MHZ, MeOD) δ 8.40 (t, J=1.3 Hz, 1H), 8.04-7.89 (m, 5H), 7.59 (dd, J=9.5, 1.5 Hz, 1H), 7.46 (d, J=7.9 Hz, 1H), 7.44-7.37 (m, 2H), 2.40 (s, 3H).

### Example 1. N-(2-(Diethylamino)ethyl)-3-methyl-4-(3-(3-(trifluoromethyl)phenyl)-imidazo[1,5-a]pyridin-6-yl)benzamide

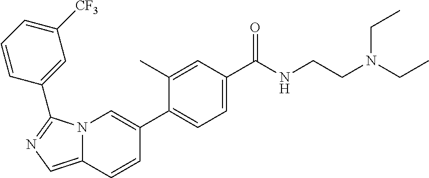 [SMILES: CCN(CC)CCNC(=O)c1ccc(-c2ccc3cnc(-c4cccc(C(F)(F)F)c4)n3c2)c(C)c1]

General procedure D1 was employed using 3-methyl-4-(3-(3-(trifluoromethyl)phenyl)-imidazo[1,5-a]pyridin-6-yl)benzoic acid (50 mg, 0.13 mmol, 1 eq.). A yellow solid (36 mg, 58%) was isolated by CombiFlash chromatography (silica gel, MeOH/CH₂Cl₂, 3:17). ¹H NMR (400 MHZ, MeOD) δ 8.27 (t, J=1.2 Hz, 1H), 8.15-8.07 (m, 2H), 7.84-7.71 (m, 5H), 7.67 (s, 1H), 7.43 (d, J=7.9 Hz, 1H), 6.95 (dd, J=9.3, 1.3 Hz, 1H), 3.56 (t, J=7.3 Hz, 2H), 2.82 (t, J=7.0 Hz, 2H), 2.75 (q, J=7.2 Hz, 4H), 2.42 (s, 3H), 1.15 (t, J=7.2 Hz, 6H).

### Example 2. N-(2-(Diethylamino)ethyl)-3-methyl-4-(3-(3-methylphenyl)-imidazo[1,5-a]pyridin-6-yl)benzamide

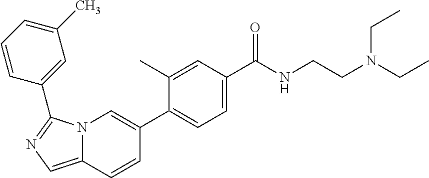 [SMILES: CCN(CC)CCNC(=O)c1ccc(-c2ccc3cnc(-c4cccc(C)c4)n3c2)c(C)c1]

General procedure D1 was employed using 3-methyl-4-(3-(3-methylphenyl)imidazo-[1,5-a]pyridin-6-yl)benzoic acid (60 mg, 0.18 mmol, 1 eq.). A yellow solid (49 mg, 64%) was isolated by CombiFlash chromatography (silica gel, MeOH/CH₂Cl₂, 3:17). ¹H NMR (400 MHZ, CDCl₃) δ 8.18 (dd, J=1.4 Hz, 0.75 Hz, 1H), 7.83 (s, 1H), 7.72 (d, J=7.8 Hz, 1H), 7.63 (s, 1H), 7.60 (s, 1H), 7.57 (d, J=7.7 Hz, 1H), 7.53 (dd, J=9.3, 1.2 Hz, 1H), 7.39 (t, J=7.6 Hz, 1H), 7.32 (d, J=7.9 Hz, 1H), 7.23 (d, J=7.6 Hz, 1H), 6.72 (dd, J=9.3, 1.4 Hz, 1H), 3.71-3.55 (m, 2H), 3.00-2.62 (m, 6H), 2.43 (s, 3H), 2.39 (s, 3H), 1.30-1.03 (m, 6H).

### Example 3. N-(2-(Dimethylamino)ethyl)-4-(3-(4-tolyl)-[1,2,4]triazolo[4,3-b]pyridazin-6-yl)benzamide

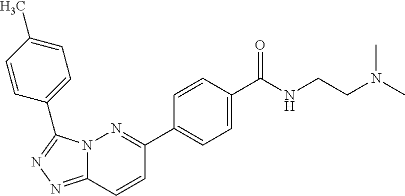 [SMILES: Cc1ccc(-c2nnc3ccc(-c4ccc(C(=O)NCCN(C)C)cc4)nn23)cc1]

General procedure D5 was employed using 4-(3-(4-tolyl)-[1,2,4]triazolo[4,3-b]pyridazin-6-yl)benzoic acid (30 mg, 0.091 mmol, 1 eq.) and N¹,N¹-dimethylethane-1,2-diamine (20 μL, 0.18 mmol, 2 eq.). A white solid (22 mg, 60%) was isolated by CombiFlash chromatography (silica gel, MeOH/CH₂Cl₂, 1:4). ¹H NMR (400 MHZ, CDCl₃) δ 8.48 (dd, J=8.2, 1.8 Hz, 2H), 8.29 (dd, J=9.7 Hz, 1H), 8.12 (dd, J=8.7 Hz, 1.9 Hz, 2H), 8.04 (dd, J=8.6 Hz, 2.3 Hz, 2H), 7.64 (d, J=9.7 Hz, 1H), 7.43 (d, J=8.0 Hz, 2H), 7.14 (s, 1H), 3.62 (q, J=5.4 Hz, 2H), 2.64 (t, J=5.7 Hz, 2H), 2.50 (s, 3H), 2.37 (s, 6H).

### Example 4. N-(2-(Dimethylamino)ethyl)-3-methyl-4-(3-(4-tolyl)-[1,2,4]triazolo[4,3-b]pyridazin-6-yl)benzamide

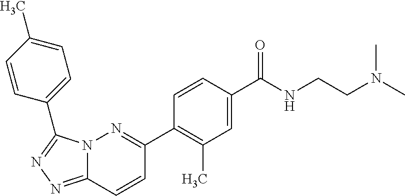 [SMILES: Cc1ccc(-c2nnc3ccc(-c4ccc(C(=O)NCCN(C)C)cc4C)nn23)cc1]

General procedure D5 was employed using 3-methyl-4-(3-(4-tolyl)-[1,2,4]triazolo[4,3-b]pyridazin-6-yl)benzoic acid (50 mg, 0.15 mmol, 1 eq.) and N¹,N¹-dimethylethane-1,2-diamine (31 μL, 0.29 mmol, 2 eq.). A white solid (33 mg, 55%) was isolated by CombiFlash chromatography (silica gel, MeOH/CH₂Cl₂, 1:4). ¹H NMR (400 MHZ, MeOD) δ 8.38 (d, J=9.6 Hz, 1H), 8.31 (dd, J=8.7 Hz, 2.0 Hz, 2H), 7.92 (d, J=1.8 Hz, 1H), 7.87 (dd, J=8.0, 1.9 Hz, 1H), 7.70 (d, J=8.0 Hz, 1H), 7.63 (d, J=9.6 Hz, 1H), 7.42 (d, J=8.0 Hz, 2H), 3.61 (t, J=6.7 Hz, 2H), 2.69 (t, J=6.7 Hz, 2H), 2.55 (s, 3H), 2.46 (s, 3H), 2.41 (s, 6H).

### Example 5. N-(2-(Dimethylamino)ethyl)-2-methyl-4-(3-(4-tolyl)-[1,2,4]triazolo[4,3-b]pyridazin-6-yl)benzamide

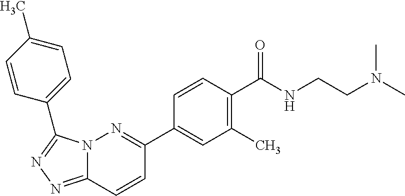 [SMILES: Cc1ccc(-c2nnc3ccc(-c4ccc(C(=O)NCCN(C)C)c(C)c4)nn23)cc1]

General procedure D5 was employed using 2-methyl-4-(3-(4-tolyl)-[1,2,4]triazolo[4,3-b]pyridazin-6-yl)benzoic acid (50 mg, 0.15 mmol, 1 eq.) and N¹,N¹-dimethylethane-1,2-diamine (32 μL, 0.29 mmol, 2 eq.). A white solid (39 mg, 64%) was isolated by CombiFlash chromatography (silica gel, MeOH/CH₂Cl₂, 1:4). ¹H NMR (400 MHZ, MeOD) δ 8.40-8.35 (m, 3H), 8.07-7.99 (m, 3H), 7.62 (d, J=8.4 Hz, 1H), 7.48 (d, J=8.0 Hz, 2H), 3.58 (t, J=6.9 Hz, 2H), 2.67 (t, J=6.9 Hz, 2H), 2.56 (s, 3H), 2.50 (s, 3H), 2.39 (s, 6H).

### Example 6. N-(2-(Dimethylamino)ethyl)-2-methoxy-4-(3-(4-tolyl)-[1,2,4]triazolo[4,3-b]pyridazin-6-yl)benzamide

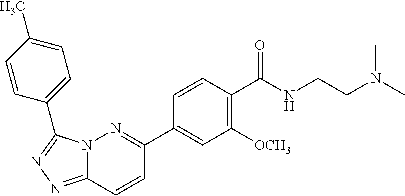 [SMILES: COc1cc(-c2ccc3nnc(-c4ccc(C)cc4)n3n2)ccc1C(=O)NCCN(C)C]

General procedure D5 was employed using 2-methoxy-4-(3-(4-tolyl)-[1,2,4]triazolo[4,3-b]pyridazin-6-yl)benzoic acid (80 mg, 0.22 mmol, 1 eq.) and N¹,N¹-dimethylethane-1,2-diamine (48 μL, 0.44 mmol, 2 eq.). A white solid (56 mg, 59%) was isolated by CombiFlash chromatography (silica gel, MeOH/CH₂Cl₂, 1:4). ¹H NMR (400 MHZ, MeOD) δ 8.39 (dd, J=9.1, 3.0 Hz, 3H), 8.13 (d, J=8.1 Hz, 1H), 8.06 (d, J=9.8 Hz, 1H), 7.90 (d, J=1.6 Hz, 1H), 7.84 (dd, J=8.1, 1.6 Hz, 1H), 7.48 (d, J=8.1 Hz, 2H), 4.14 (s, 3H), 3.62 (t, J=6.6 Hz, 2H), 2.70 (t, J=6.6 Hz, 2H), 2.50 (s, 3H), 2.42 (s, 6H).

### Example 7. N-(2-(Diethylamino)ethyl)-4-(3-(4-tolyl)-[1,2,4]triazolo[4,3-b]pyridazin-6-yl)benzamide

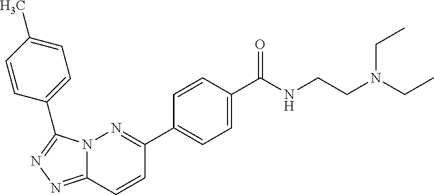 [SMILES: CCN(CC)CCNC(=O)c1ccc(-c2ccc3nnc(-c4ccc(C)cc4)n3n2)cc1]

General procedure D5 was employed using 4-(3-(4-tolyl)-[1,2,4]triazolo[4,3-b]pyridazin-6-yl)benzoic acid (30 mg, 0.091 mmol, 1 eq.) and N¹,N¹-diethylethane-1,2-diamine (26 μL, 0.18 mmol, 2 eq.). A white solid (25 mg, 64%) was isolated by CombiFlash chromatography (silica gel, MeOH/CH₂Cl₂, 1:4). ¹H NMR (400 MHZ, CDCl₃) δ 8.48 (dd, J=8.2 Hz, 1.9 Hz, 2H), 8.29 (d, J=9.7 Hz, 1H), 8.12 (dd, J=8.2 Hz, 1.6 Hz, 2H), 8.01 (dd, J=8.3 Hz, 1.8 Hz 2H), 7.64 (d, J=9.7 Hz, 1H), 7.44 (d, J=8.0 Hz, 2H), 7.12 (s, 1H), 3.56 (q, J=5.4 Hz, 2H), 2.73 (t, J=5.9 Hz, 2H), 2.63 (q, J=7.1 Hz, 4H), 2.50 (s, 3H), 1.10 (t, J=7.1 Hz, 6H).

### Example 8. N-(2-(Diethylamino)ethyl)-3-methyl-4-(3-(4-tolyl)-[1,2,4]triazolo[4,3-b]pyridazin-6-yl)benzamide

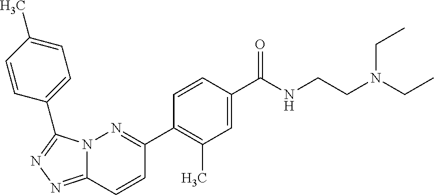 [SMILES: CCN(CC)CCNC(=O)c1ccc(-c2ccc3nnc(-c4ccc(C)cc4)n3n2)c(C)c1]

General procedure D5 was employed using 3-methyl-4-(3-(4-tolyl)-[1,2,4]triazolo[4,3-b]pyridazin-6-yl)benzoic acid (25 mg, 0.073 mmol, 1 eq.) and N¹,N¹-diethylethane-1,2-diamine (20 μL, 0.15 mmol, 2 eq.). A white solid (19 mg, 58%) was isolated by CombiFlash chromatography (silica gel, MeOH/CH₂Cl₂, 1:4). ¹H NMR (400 MHZ, CDCl₃) δ 8.40 (d, J=8.1 Hz, 2H), 8.23 (d, J=9.5 Hz, 1H), 7.82 (d, J=1.8 Hz, 1H), 7.73 (dd, J=8.0, 1.8 Hz, 1H), 7.56 (d, J=8.0 Hz, 1H), 7.34 (d, J=8.0 Hz, 2H), 7.27 (d, J=9.6 Hz, 1H), 7.05 (s, 1H), 3.53 (q, J=5.5 Hz, 2H), 2.70 (t, J=6.0 Hz, 2H), 2.61 (q, J=7.1 Hz, 4H), 2.52 (s, 3H), 2.43 (s, 3H), 1.07 (t, J=7.1 Hz, 6H).

### Example 9. N-(2-(Diethylamino)ethyl)-2-methyl-4-(3-(4-tolyl)-[1,2,4]triazolo[4,3-b]pyridazin-6-yl)benzamide

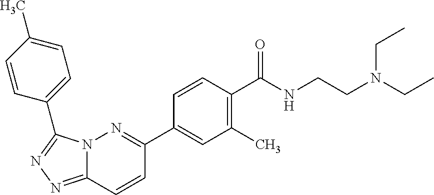 [SMILES: CCN(CC)CCNC(=O)c1ccc(-c2ccc3nnc(-c4ccc(C)cc4)n3n2)cc1C]

General procedure D5 was employed using 2-methyl-4-(3-(4-tolyl)-[1,2,4]triazolo[4,3-b]pyridazin-6-yl)benzoic acid (16 mg, 0.046 mmol, 1 eq.) and N¹,N¹-diethylethane-1,2-diamine (13 μL, 0.093 mmol, 2 eq.). A white solid (10 mg, 51%) was isolated by CombiFlash chromatography (silica gel, MeOH/CH₂Cl₂, 1:4). ¹H NMR (400 MHZ, DMSO-d₆) δ 8.55 (d, J=9.8 Hz, 1H), 8.37 (d, J=8.2 Hz, 2H), 8.30 (t, J=5.7 Hz, 1H), 8.06-7.98 (m, 3H), 7.53 (d, J=7.9 Hz, 1H), 7.48 (d, J=8.0 Hz, 2H), 2.64-2.54 (m, 6H), 2.50-2.46 (m, 5H), 2.44 (s, 3H), 1.01 (t, J=7.1 Hz, 6H).

### Example 10. N-(2-(Diethylamino)ethyl)-2-methoxy-4-(3-(4-tolyl)-[1,2,4]triazolo[4,3-b]pyridazin-6-yl)benzamide

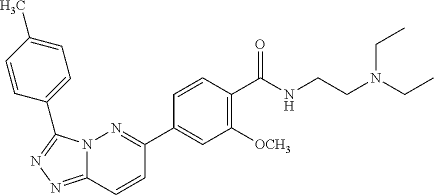 [SMILES: CCN(CC)CCNC(=O)c1ccc(-c2ccc3nnc(-c4ccc(C)cc4)n3n2)cc1OC]

General procedures D5 was employed using 2-methoxy-4-(3-(4-tolyl)-[1,2,4]triazolo[4,3-b]pyridazin-6-yl)benzoic acid (40 mg, 0.12 mmol, 1 eq.) and N¹,N¹-diethylethane-1,2-diamine (33 μL, 0.23 mmol, 2 eq.). A white solid (31 mg, 61%) was isolated by CombiFlash chromatography (silica gel, MeOH/CH₂Cl₂, 1:4). ¹H NMR (400 MHZ, CDCl₃) δ 8.47 (d, J=8.3 Hz, 2H), 8.34 (d, J=8.0 Hz, 1H), 8.27 (d, J=9.8 Hz, 1H), 7.72 (d, J=1.6 Hz, 1H), 7.66-7.59 (m, 2H), 7.40 (d, J=8.0 Hz, 2H), 4.15 (s, 3H), 3.90-3.76 (m, 2H), 3.15-2.79 (m, 6H), 2.48 (s, 3H), 1.31-1.27 (m, 6H).

### Example 11. N-(2-(Dimethylamino)ethyl)-1-(3-(4-tolyl)-[1,2,4]triazolo[4,3-b]pyridazin-6-yl)piperidine-4-carboxamide

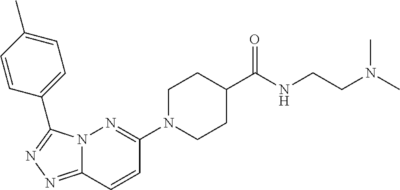 [SMILES: Cc1ccc(-c2nnc3ccc(N4CCC(C(=O)NCCN(C)C)CC4)nn23)cc1]

General procedure F was employed using methyl 1-(3-(4-tolyl)-[1,2,4]triazolo[4,3-b]pyridazin-6-yl)piperidine-4-carboxylate (20 mg, 0.057 mmol, 1 eq.) and N¹,N¹-dimethylethane-1,2-diamine (1 mL). A white solid (17 mg, 72%) was isolated by CombiFlash chromatography (C18, ACN/Water, 1:1). ¹H NMR (400 MHZ, MeOD) δ 8.29 (d, J=8.3 Hz, 2H), 7.98 (d, J=10.2 Hz, 1H), 7.45-7.38 (m, 3H), 4.38 (d, J=13.5 Hz, 2H), 3.13 (td, J=13.0 Hz, 1.9 Hz, 2H), 2.56 (tt, J=11.5, 3.9 Hz, 1H), 2.47 (d, J=9.1 Hz, 4H), 2.30 (s, 6H), 1.95 (dd, J=13.1 Hz, 3.3 Hz, 2H), 1.83 (qd, J=12.5 Hz, 3.9 Hz, 2H).

### Example 12. N-(2-(Dimethylamino)ethyl)-1-(3-4-tolyl)-[1,2,4]triazolo[4,3-b]pyridazin-6-yl)piperidine-3-carboxamide

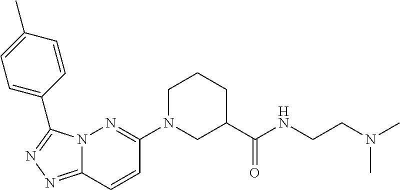 [SMILES: Cc1ccc(-c2nnc3ccc(N4CCCC(C(=O)NCCN(C)C)C4)nn23)cc1]

General procedure F was employed using methyl 1-(3-(4-tolyl)-[1,2,4]triazolo[4,3-b]pyridazin-6-yl)piperidine-3-carboxylate (20 mg, 0.057 mmol, 1 eq.) and N¹,N¹-dimethylethane-1,2-diamine (1 mL). A white solid (13 mg, 57%) was isolated by CombiFlash chromatography (C18, ACN/Water, 1:1). ¹H NMR (400 MHZ, CDCl₃) δ 8.40 (dd, J=8.1 Hz, 1.8 Hz, 2H), 7.93 (d, J=10.1 Hz, 1H), 7.35 (d, J=8.0 Hz, 2H), 7.02 (d, J=10.1 Hz, 1H), 6.37 (s, 1H), 4.20 (dd, J=14.4 Hz, 3.6 Hz, 1H), 4.03 (dt, J=13.4 Hz, 3.6 Hz, 1H), 3.47-3.34 (m, 3H), 3.30-3.21 (m, 1H), 2.56 (tt, J=10.1, 5.0 Hz, 1H), 2.48-2.41 (m, 5H), 2.21 (s, 6H), 2.08-1.85 (m, 4H).

### Example 13. N-(2-(Dimethylamino)ethyl)-1-(3-(4-tolyl)-[1,2,4]triazolo[4,3-b]pyridazin-6-yl)pyrrolidine-3-carboxamide

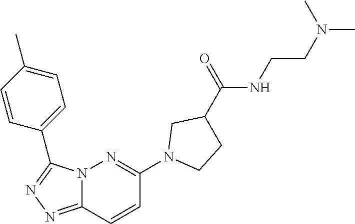 [SMILES: Cc1ccc(-c2nnc3ccc(N4CCC(C(=O)NCCN(C)C)C4)nn23)cc1]

General procedure F was employed using methyl 1-(3-(4-tolyl)-[1,2,4]triazolo[4,3-b]pyridazin-6-yl)pyrrolidine-3-carboxylate (20 mg, 0.059 mmol, 1 eq.) and N¹,N¹-dimethylethane-1,2-diamine (1 mL). A white solid (16 mg, 69%) was isolated by CombiFlash chromatography (C18, ACN/Water, 1:1). ¹H NMR (400 MHZ, CDCl₃) δ 8.46 (d, J=8.2 Hz, 2H), 7.91 (d, J=9.9 Hz, 1H), 7.35 (d, J=8.0 Hz, 2H), 6.73 (d, J=10.0 Hz, 1H), 6.54 (s, 1H), 3.87-3.77 (m, 3H), 3.59 (dt, J=9.9 Hz, 7.5 Hz, 1H), 3.42 (q, J=5.4 Hz, 2H), 3.10 (p, J=7.6 Hz, 1H), 2.52 (t, J=5.8 Hz, 2H), 2.45 (s, 3H), 2.39-2.27 (m, 8H).

### Example 14. N-(2-(Diethylamino)ethyl)-1-(3-(4-tolyl)-[1,2,4]triazolo[4,3-b]pyridazin-6-yl)piperidine-4-carboxamide

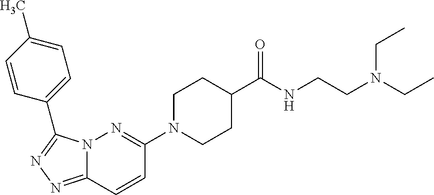 [SMILES: CCN(CC)CCNC(=O)C1CCN(c2ccc3nnc(-c4ccc(C)cc4)n3n2)CC1]

General procedure F was employed using methyl 1-(3-(4-tolyl)-[1,2,4]triazolo[4,3-b]pyridazin-6-yl)piperidine-4-carboxylate (90 mg, 0.26 mmol, 1 eq.) and N¹,N¹-diethylethane-1,2-diamine (4.5 mL). A white solid (86 mg, 77%) was isolated by CombiFlash chromatography (C18, ACN/Water, 1:1). ¹H NMR (400 MHZ, CDCl₃) δ 8.37 (d, J=7.9 Hz, 2H), 7.92 (d, J=10.2 Hz, 1H), 7.34 (d, J=7.9 Hz, 2H), 6.97 (d, J=10.2 Hz, 1H), 6.36 (s, 1H), 4.23 (d, J=13.2 Hz, 2H), 3.31 (q, J=5.6 Hz, 2H), 3.09 (t, J=12.6 Hz, 2H), 2.70-2.47 (m, 6H), 2.46-2.35 (m, 4H), 2.07-1.97 (m, 2H), 1.87 (qd, J=12.1 Hz, 3.7 Hz, 2H), 1.02 (t, J=7.1 Hz, 6H).

### Example 15. N-(2-(Diethylamino)ethyl)-1-(3-(3-tolyl)-[1,2,4]triazolo[4,3-b]pyridazin-6-yl)piperidine-4-carboxamide

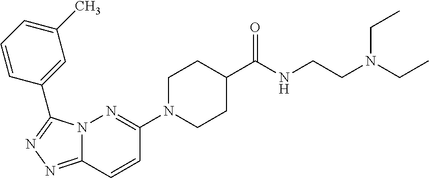 [SMILES: CCN(CC)CCNC(=O)C1CCN(c2ccc3nnc(-c4cccc(C)c4)n3n2)CC1]

General procedure F was employed using methyl 1-(3-(3-tolyl)-[1,2,4]triazolo[4,3-b]pyridazin-6-yl)piperidine-4-carboxylate (40 mg, 0.11 mmol, 1 eq.) and N¹,N¹-diethylethane-1,2-diamine (2 mL). A white solid (34 mg, 68%) was isolated by CombiFlash chromatography (C18, ACN/Water, 1:1). ¹H NMR (400 MHZ, acetone-d₆) δ 8.40 (s, 1H), 8.36 (d, J=7.8 Hz, 1H), 8.00 (d, J=10.1 Hz, 1H), 7.45 (t, J=7.7 Hz, 1H), 7.38 (d, J=10.2 Hz, 1H), 7.33 (d, J=7.7 Hz, 1H), 6.96 (br s, 1H), 4.36 (dt, J=13.4, 3.4 Hz, 2H), 3.28-3.15 (m, 4H), 2.62-2.48 (m, 7H), 2.46 (s, 3H), 1.99-1.80 (m, 4H), 0.99 (t, J=7.1 Hz, 6H).

### Example 16. N-(2-(Diethylamino)ethyl)-1-(3-(4-fluorophenyl)-[1,2,4]triazolo[4,3-b]pyridazin-6-yl)piperidine-4-carboxamide

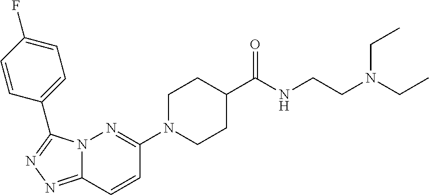 [SMILES: CCN(CC)CCNC(=O)C1CCN(c2ccc3nnc(-c4ccc(F)cc4)n3n2)CC1]

General procedure F was employed using methyl 1-(3-(4-fluorophenyl)-[1,2,4]triazolo[4,3-b]pyridazin-6-yl)piperidine-4-carboxylate (40 mg, 0.11 mmol, 1 eq.) and N¹,N¹-diethylethane-1,2-diamine (2 mL). A white solid (28 mg, 57%) was isolated by CombiFlash chromatography (C18, ACN/Water, 1:1). ¹H NMR (400 MHZ, CDCl₃) δ 8.53-8.46 (m, 2H), 7.93 (d, J=10.1 Hz, 1H), 7.21 (tt, J=8.7 Hz, 2.0 Hz, 2H), 6.99 (d, J=10.2 Hz, 1H), 6.37 (s, 1H), 4.22 (d, J=13.4 Hz, 2H), 3.31 (q, J=5.4 Hz, 2H), 3.11 (td, J=13.4, 2.9 Hz, 2H), 2.75 (t, J=6.3 Hz, 2H), 2.53-2.46 (m, 4H), 2.46-2.36 (m, 1H), 2.02 (dd, J=13.7, 3.5 Hz, 2H), 1.90 (qd, 12.6 Hz, 3.9 Hz, 2H), 1.02 (t, J=7.1 Hz, 6H).

### Example 17. N-(2-(Diethylamino)ethyl)-1-(3-(4-methoxyphenyl)-[1,2,4]triazolo[4,3-b]pyridazin-6-yl)piperidine-4-carboxamide

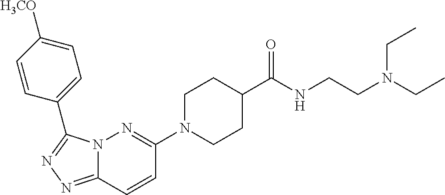 [SMILES: CCN(CC)CCNC(=O)C1CCN(c2ccc3nnc(-c4ccc(OC)cc4)n3n2)CC1]

General procedure F was employed using methyl 1-(3-(4-methoxyphenyl)-[1,2,4]triazolo[4,3-b]pyridazin-6-yl)piperidine-4-carboxylate (40 mg, 0.11 mmol, 1 eq.) and N¹,N¹-diethylethane-1,2-diamine (2 mL). A white solid (25 mg, 51%) was isolated by CombiFlash chromatography (C18, ACN/Water, 1:1). ¹H NMR (400 MHZ, CDCl₃) δ 8.44 (dt, J=8.9 Hz, 2.8 Hz, 2H), 7.91 (d, J=10.1 Hz, 1H), 7.05 (dt, J=9.0 Hz, 2.1 Hz, 2H), 6.96 (d, J=10.2 Hz, 1H), 6.34 (s, 1H), 4.22 (d, J=13.4 Hz, 2H), 3.89 (s, 3H), 3.33 (dt, J=4.8 Hz, 6.3 Hz, 2H), 3.15-3.03 (m, 2H), 2.70-2.49 (m, 6H), 2.47-2.36 (m, 1H), 2.03 (dd, J=13.3 Hz, 3.3 Hz, 2H), 1.94-1.80 (m, 2H), 1.04 (t, J=6.0 Hz, 6H).

### Example 18. N-(2-(Diethylamino)ethyl)-1-(3-(4-(trifluoromethyl)phenyl)-[1,2,4]triazolo[4,3-b]pyridazin-6-yl)piperidine-4-carboxamide

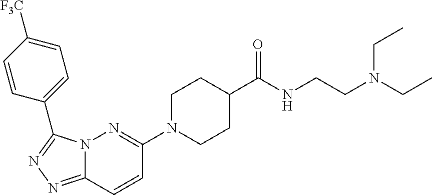 [SMILES: CCN(CC)CCNC(=O)C1CCN(c2ccc3nnc(-c4ccc(C(F)(F)F)cc4)n3n2)CC1]

General procedure F was employed using methyl 1-(3-(4-(trifluoromethyl)phenyl)-[1,2,4]-triazolo[4,3-b]pyridazin-6-yl)piperidine-4-carboxylate (50 mg, 0.12 mmol, 1 eq.) and N¹,N¹-diethylethane-1,2-diamine (2.5 mL). A white solid (42 mg, 70%) was isolated by CombiFlash chromatography (C18, ACN/Water, 1:1). ¹H NMR (400 MHZ, CDCl₃) δ 8.65 (d, J=8.2 Hz, 2H), 8.46 (s, 1H), 7.95 (d, J=10.1 Hz, 1H), 7.80 (d, J=8.3 Hz, 2H), 7.02 (d, J=10.1 Hz, 1H), 4.24 (d, J=13.5 Hz, 2H), 3.67 (q, J=5.4 Hz, 2H), 3.19-3.11 (m, 6H), 3.01-2.89 (m, 2H), 2.70-2.60 (m, 1H), 2.11 (d, J=13.2 Hz, 2H), 1.90 (qd, J=12.9 Hz, 3.9 Hz, 2H), 1.44 (t, J=7.3 Hz, 6H).

### Example 19. N-(2-(Diethylamino)ethyl)-1-(3-(4-chlorophenyl)-[1,2,4]triazolo[4,3-b]pyridazin-6-yl)piperidine-4-carboxamide

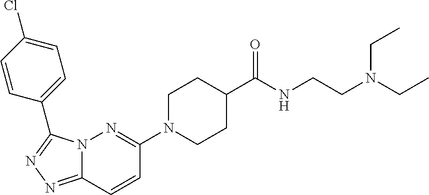 [SMILES: CCN(CC)CCNC(=O)C1CCN(c2ccc3nnc(-c4ccc(Cl)cc4)n3n2)CC1]

General procedure F was employed using methyl 1-(3-(4-chlorophenyl)-[1,2,4]triazolo[4,3-b]pyridazin-6-yl)piperidine-4-carboxylate (40 mg, 0.11 mmol, 1 eq.) and N¹,N¹-diethylethane-1,2-diamine (2 mL). A white solid (34 mg, 73%) was isolated by CombiFlash chromatography (C18, ACN/Water, 1:1). ¹H NMR (400 MHZ, CDCl₃) δ 8.45 (dt, J=8.7 Hz, 2.7 Hz, 2H), 7.93 (d, J=10.1 Hz, 1H), 7.50 (dt, J=8.7 Hz, 2.5 Hz, 2H), 7.00 (d, J=10.1 Hz, 1H), 6.31 (s, 1H), 4.22 (dt, J=13.5, 3.7 Hz, 2H), 3.31 (q, J=5.5 Hz, 2H), 3.11 (td, J=12.2 Hz, 2.5 Hz, 2H), 2.60-2.49 (m, 6H), 2.42 (tt, J=11.2, 4.0 Hz, 1H), 2.06-1.97 (m, 2H), 1.88 (qd, J=12.6 Hz, 4.0 Hz, 2H), 1.02 (t, J=7.1 Hz, 6H).

### Example 20. 1-(3-(3,4-Dichlorophenyl)-[1,2,4]triazolo[4,3-b]pyridazin-6-yl)-N-(2-(diethylamino)ethyl)piperidine-4-carboxamide

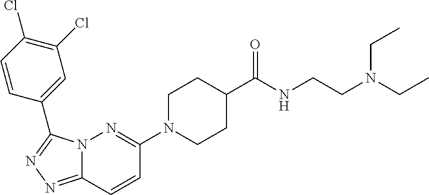 [SMILES: CCN(CC)CCNC(=O)C1CCN(c2ccc3nnc(-c4ccc(Cl)c(Cl)c4)n3n2)CC1]

General procedure F was employed using methyl 1-[3-(3,4-dichlorophenyl)-1,2,3a,4-tetraaza-5-indenyl]-4-piperidinecarboxylate (40 mg, 0.099 mmol, 1 eq.) and N¹,N¹-diethylethane-1,2-diamine (2 mL). A white solid (34 mg, 70%) was isolated by CombiFlash chromatography (C18, ACN/Water, 1:1). ¹H NMR (400 MHZ, CDCl₃) δ 8.74 (d, J=2.0 Hz, 1H), 8.36 (dd, J=8.5, 2.0 Hz, 1H), 7.94 (d, J=10.1 Hz, 1H), 7.60 (d, J=8.5 Hz, 1H), 7.02 (d, J=10.2 Hz, 1H), 4.24 (d, J=13.4 Hz, 2H), 3.31 (q, J=5.5 Hz, 2H), 3.20-3.10 (m, 2H), 2.58-2.51 (m, 6H), 2.50-2.43 (m, 1H), 2.03 (dd, J=13.5, 3.5 Hz, 2H), 1.90 (qd, J=12.4, 4.0 Hz, 2H), 1.02 (t, J=7.1 Hz, 6H).

### Example 21. N-(2-(Diethylamino)ethyl)-1-(3-(2-tolyl)-[1,2,4]triazolo[4,3-b]pyridazin-6-yl)piperidine-4-carboxamide

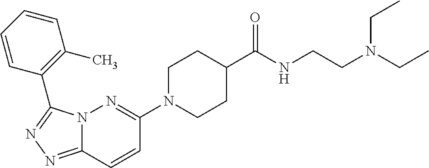 [SMILES: CCN(CC)CCNC(=O)C1CCN(c2ccc3nnc(-c4ccccc4C)n3n2)CC1]

General procedure F was employed using methyl 1-[3-(2-tolyl)-1,2,3a,4-tetraaza-5-indenyl]-4-piperidinecarboxylate (40 mg, 0.11 mmol, 1 eq.) and N¹,N¹-diethylethane-1,2-diamine (2 mL). A white solid (22 mg, 42%) was isolated by CombiFlash chromatography (C18, ACN/Water, 1:1). ¹H NMR (400 MHZ, CDCl₃) δ 7.92 (d, J=10.1 Hz, 1H), 7.71 (dd, J=7.5, 1.5 Hz, 1H), 7.44-7.29 (m, 3H), 6.98 (d, J=10.1 Hz, 1H), 6.25 (s, 1H), 4.13 (d, J=13.1 Hz, 2H), 3.28 (q, J=5.5 Hz, 2H), 2.97 (td, J=12.6 Hz, 2.9 Hz, 2H), 2.57-2.48 (m, 6H), 2.43 (s, 3H), 2.35 (tt, J=11.4, 3.9 Hz, 1H), 1.93 (dd, J=13.4, 3.4 Hz, 2H), 1.80 (qd, 13.3 Hz, 4.1 Hz, 2H), 1.00 (t, J=7.1 Hz, 6H).

### Example 22. N-(2-(Diethylamino)ethyl)-1-(3-(3-(trifluoromethyl)phenyl)-[1,2,4]triazolo[4,3-b]pyridazin-6-yl)piperidine-4-carboxamide

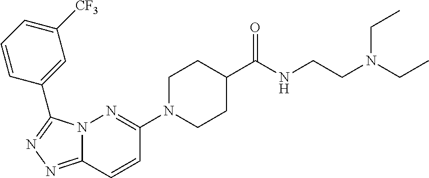 [SMILES: CCN(CC)CCNC(=O)C1CCN(c2ccc3nnc(-c4cccc(C(F)(F)F)c4)n3n2)CC1]

General procedure F was employed using methyl 1-{3-[3-(trifluoromethyl)phenyl]-1,2,3a,4-tetraaza-5-indenyl}-4-piperidinecarboxylate (40 mg, 0.099 mmol, 1 eq.) and N¹,N¹-diethylethane-1,2-diamine (2 mL). A white solid (42 mg, 87%) was isolated by CombiFlash chromatography (C18, ACN/Water, 1:1). ¹H NMR (400 MHZ, CDCl₃) δ 8.98 (s, 1H), 8.69 (d, J=7.7 Hz, 1H), 7.95 (d, J=10.1 Hz, 1H), 7.68 (dt, J=15.6, 7.8 Hz, 2H), 7.02 (d, J=10.2 Hz, 1H), 4.25 (d, J=13.6 Hz, 2H), 3.66-3.53 (m, 2H), 3.25-2.88 (m, 8H), 2.69-2.57 (m, 1H), 2.06 (d, J=12.7 Hz, 2H), 1.90 (qd, J=12.1, 4.0 Hz, 2H), 1.44-1.29 (m, 6H).

### Example 23. N-(2-(Diethylamino)ethyl)-1-(3-(3-methoxyphenyl)-[1,2,4]triazolo[4,3-b]pyridazin-6-yl)piperidine-4-carboxamide

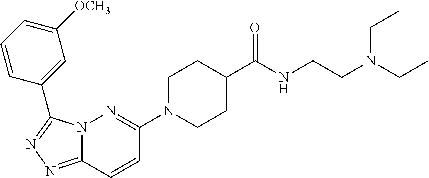 [SMILES: CCN(CC)CCNC(=O)C1CCN(c2ccc3nnc(-c4cccc(OC)c4)n3n2)CC1]

General procedure F was employed using methyl 1-[3-(3-methoxyphenyl)-1,2,3a,4-tetraaza-5-indenyl]-4-piperidinecarboxylate (40 mg, 0.11 mmol, 1 eq.) and N¹,N¹-diethylethane-1,2-diamine (2 mL). A white solid (25 mg, 51%) was isolated by CombiFlash chromatography (C18, ACN/Water, 1:1). ¹H NMR (400 MHZ, CDCl₃) δ 8.16-8.07 (m, 2H), 7.93 (d, J=10.2 Hz, 1H), 7.43 (t, J=8.0 Hz, 1H), 7.03 (dd, J=8.5, 2.6 Hz, 1H), 6.99 (d, J=10.1 Hz, 1H), 6.29 (s, 1H), 4.25 (d, J=13.4 Hz, 2H), 3.90 (s, 3H), 3.31 (q, J=5.6 Hz, 2H), 3.11 (td, J=14.0, 2.9 Hz, 2H), 2.65-2.43 (m, 6H), 2.47-2.35 (m, 1H), 2.02 (d, J=13.1 Hz, 2H), 1.90 (qd, J=12.3 Hz, 3.9 Hz, 2H), 1.02 (t, J=7.1 Hz, 6H).

### Example 24. 1-(3-(3-Chlorophenyl)-[1,2,4]triazolo[4,3-b]pyridazin-6-yl)-N-(2-(diethylamino)ethyl)piperidine-4-carboxamide

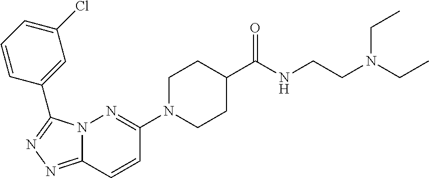 [SMILES: CCN(CC)CCNC(=O)C1CCN(c2ccc3nnc(-c4cccc(Cl)c4)n3n2)CC1]

General procedure F was employed using methyl 1-[3-(3-methoxyphenyl)-1,2,3a,4-tetraaza-5-indenyl]-4 piperidinecarboxylate (40 mg, 0.11 mmol, 1 eq.) and N¹,N¹-diethylethane-1,2-diamine (2 mL). A white solid (27 mg, 54%) was isolated by CombiFlash chromatography (C18, ACN/Water, 1:1). ¹H NMR (400 MHZ, CDCl₃) δ 8.59 (t, J=1.8 Hz, 1H), 8.40 (dt, J=7.3, 1.7 Hz, 1H), 7.93 (d, J=10.1 Hz, 1H), 7.50-7.41 (m, 2H), 7.00 (d, J=10.2 Hz, 1H), 4.24 (dt, J=13.4, 3.7 Hz, 2H), 3.55-3.41 (m, 2H), 3.14 (td, J=12.9 Hz, 2.9 Hz, 2H), 2.96-2.67 (m, 6H), 2.59-2.48 (m, 1H), 2.06 (dd, J=13.5, 3.6 Hz, 2H), 1.97-1.82 (qd, J=13.0 Hz, 3.8 Hz, 2H), 1.20 (t, J=7.7 Hz, 6H).

### Example 25. N-(2-(Diethylamino)ethyl)-1-(3-(4-tolyl)-[1,2,4]triazolo[4,3-b]pyridazin-6-yl)piperidine-3-carboxamide

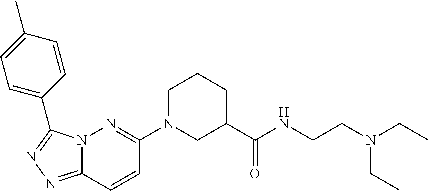 [SMILES: CCN(CC)CCNC(=O)C1CCCN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)piperidine-3-carboxylate (20 mg, 0.057 mmol, 1 eq.) and N¹,N¹-diethylethane-1,2-diamine (1 mL). A white solid 16 mg, 64%) was isolated by CombiFlash chromatography (C18, ACN/Water, 1:1). ¹H NMR (400 MHZ, CDCl₃) δ 8.37 (d, J=8.3 Hz, 2H), 7.90 (d, J=10.1 Hz, 1H), 7.33 (d, J=8.0 Hz, 2H), 7.00 (d, J=10.1 Hz, 1H), 6.40 (s, 1H), 4.16 (dd, J=14.2 Hz, 3.8 Hz, 1H), 4.00 (dt, J=13.3 Hz, 3.9 Hz, 1H), 3.45-3.17 (m, 4H), 2.60-2.45 (m, 6H), 2.43 (s, 3H), 2.02 (dd, J=13.2, 4.6 Hz, 1H), 1.95-1.82 (m, 2H), 1.75-1.66 (m, 2H), 0.96 (t, J=7.1 Hz, 6H).

### Example 26. N-(2-(Diethylamino)ethyl)-1-(3-(4-tolyl)-[1,2,4]triazolo[4,3-b]pyridazin-6-yl)pyrrolidine-3-carboxamide

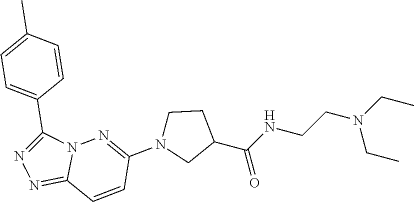 [SMILES: CCN(CC)CCNC(=O)C1CCN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)pyrrolidine-3-carboxylate (50 mg, 0.15 mmol, 1 eq.) and N¹,N¹-diethylethane-1,2-diamine (2.5 mL). A white solid (45 mg, 71%) was isolated by CombiFlash chromatography (C18, ACN/Water, 1:1). ¹H NMR (400 MHZ, DMSO-d₆) δ 8.37 (d, J=8.0 Hz, 2H), 8.11 (d, J=10.0 Hz, 1H), 8.00 (t, J=5.7 Hz, 1H), 7.39 (d, J=8.1 Hz, 2H), 7.09 (d, J=10.0 Hz, 1H), 3.77-3.64 (m, 2H), 3.63-3.49 (m, 2H), 3.18-3.07 (m, 3H), 2.49-2.41 (m, 6H), 2.39 (s, 3H), 2.25-2.07 (m, 2H), 0.95 (t, J=7.1 Hz, 6H).

### Example 27. N-(2-(Diethylamino)ethyl)-1-(3-(4-tolyl)-[1,2,4]triazolo[4,3-b]pyridazin-6-yl)azetidine-3-carboxamide

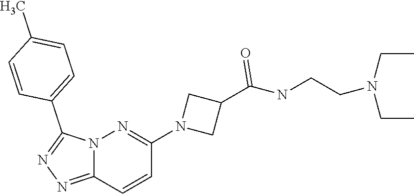 [SMILES: CCN(CC)CCNC(=O)C1CN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)azetidine-3-carboxylate (150 mg, 0.46 mmol, 1 eq.) and N¹,N¹-dimethylethane-1,2-diamine (7.5 mL). A white solid (135 mg, 71%) was isolated by CombiFlash chromatography (C18, ACN/Water, 1:1). ¹H NMR (400 MHZ, CDCl₃) δ 8.39 (dt, J=8.3 Hz, 2.1 Hz, 2H), 7.91 (d, J=9.8 Hz, 1H), 7.31 (d, J=8.1 Hz, 2H), 6.52 (d, J=9.8 Hz, 1H), 6.31 (s, 1H), 4.42-4.27 (m, 4H), 3.49 (p, J=7.3 Hz, 1H), 3.35 (q, J=5.5 Hz, 2H), 2.62-2.49 (m, 6H), 2.43 (s, 3H), 1.02 (t, J=7.1 Hz, 6H).

### Example 28. N-(2-(Diethylamino)ethyl)-1-(3-(4-fluorophenyl)-[1,2,4]triazolo[4,3-b]pyridazin-6-yl)azetidine-3-carboxamide

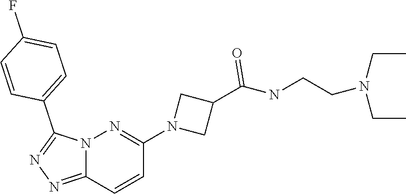 [SMILES: CCN(CC)CCNC(=O)C1CN(c2ccc3nnc(-c4ccc(F)cc4)n3n2)C1]

General procedure F was employed using methyl 1-[3-(4-fluorophenyl)-1,2,3a,4-tetraaza-5-indenyl]-3-azetidinecarboxylate (40 mg, 0.12 mmol, 1 eq.) and N¹,N¹-diethylethane-1,2-diamine (2 mL). A white solid (34 mg, 68%) was isolated by CombiFlash chromatography (C18, ACN/Water, 1:1). ¹H NMR (400 MHZ, CDCl₃) δ 8.55-8.49 (m, 2H), 7.91 (d, J=9.8 Hz, 1H), 7.19 (t, J=8.8 Hz, 2H), 6.54 (d, J=9.8 Hz, 1H), 4.42-4.29 (m, 4H), 3.61-3.51 (m, 1H), 3.49-3.39 (m, 2H), 2.78-2.56 (m, 6H), 1.12 (t, J=6.9 Hz, 6H).

### Example 29. N-(2-(Diethylamino)ethyl)-1-(3-(4-methoxyphenyl)-[1,2,4]triazolo[4,3-b]pyridazin-6-yl)azetidine-3-carboxamide

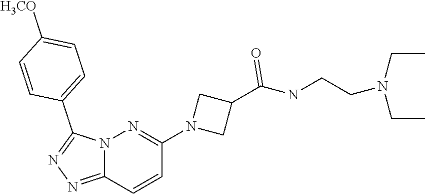 [SMILES: CCN(CC)CCNC(=O)C1CN(c2ccc3nnc(-c4ccc(OC)cc4)n3n2)C1]

General procedure F was employed using methyl 1-[3-(4-methoxyphenyl)-1,2,3a,4-tetraaza-5-indenyl]-3-azetidinecarboxylate (36 mg, 0.11 mmol, 1 eq.) and N¹,N¹-diethylethane-1,2-diamine (1 mL). A white solid (15 mg, 33%) was isolated by CombiFlash chromatography (C18, ACN/Water, 1:1). ¹H NMR (400 MHZ, CDCl₃) δ 8.71 (s, 1H), 8.47 (dt, J=8.9 Hz, 1.8 Hz, 2H), 7.89 (d, J=9.8 Hz, 1H), 7.04 (dt, J=8.9 Hz, 2.7 Hz, 2H), 6.52 (d, J=9.8 Hz, 1H), 4.37 (d, J=7.3 Hz, 4H), 3.89 (s, 3H), 3.77-3.68 (m, 3H), 3.29-3.09 (m, 6H), 1.45 (t, J=7.3 Hz, 6H).

### Example 30. N-(2-Morpholinoethyl)-1-(3-(4-tolyl)-[1,2,4]triazolo[4,3-b]pyridazin-6-yl)azetidine-3-carboxamide

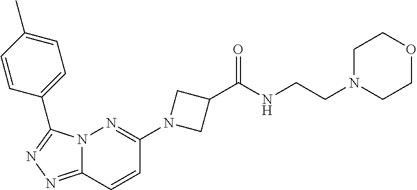 [SMILES: Cc1ccc(-c2nnc3ccc(N4CC(C(=O)NCCN5CCOCC5)C4)nn23)cc1]

General procedure F was employed using methyl 1-(3-(4-tolyl)-[1,2,4]triazolo[4,3-b]pyridazin-6-yl)azetidine-3-carboxylate (45 mg, 0.14 mmol, 1 eq.) and 2-morpholinoethan-1-amine (2 mL). A white solid (49 mg, 84%) was isolated by CombiFlash chromatography (C18, ACN/Water, 1:1). ¹H NMR (400 MHZ, CDCl₃) δ 8.22 (d, J=8.3 Hz, 2H), 7.75 (d, J=9.8 Hz, 1H), 7.15 (d, J=8.1 Hz, 2H), 6.33 (d, J=9.8 Hz, 1H), 4.23-4.11 (m, 4H), 3.71-3.54 (m, 4H), 3.42-3.27 (m, 3H), 2.75-2.29 (m, 6H), 2.27 (s, 3H).

### Example 31. N-(2-(Diisopropylamino)ethyl)-1-(3-(4-tolyl)-[1,2,4]triazolo[4,3-b]pyridazin-6-yl)azetidine-3-carboxamide

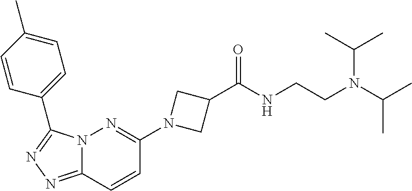 [SMILES: Cc1ccc(-c2nnc3ccc(N4CC(C(=O)NCCN(C(C)C)C(C)C)C4)nn23)cc1]

General procedure F was employed using methyl 1-(3-(4-tolyl)-[1,2,4]triazolo[4,3-b]pyridazin-6-yl)azetidine-3-carboxylate (40 mg, 0.12 mmol, 1 eq.) and N¹,N¹-diisopropylethane-1,2-diamine (2 mL). A white solid (24 mg, 40%) was isolated by CombiFlash chromatography (C18, ACN/Water, 1:1). ¹H NMR (400 MHZ, CDCl₃) δ 8.39 (d, J=8.2 Hz, 2H), 7.91 (d, J=9.8 Hz, 1H), 7.31 (d, J=8.1 Hz, 2H), 6.52 (d, J=9.8 Hz, 1H), 6.45 (s, 1H), 4.39-4.29 (m, 4H), 3.45 (d, J=8.6 Hz, 1H), 3.29 (d, J=5.9 Hz, 2H), 3.03 (p, J=6.5 Hz, 2H), 2.62 (t, J=5.9 Hz, 2H), 1.03 (d, J=6.6 Hz, 12H).

### Example 32. N-(2-(Pyrrolidin-1-yl)ethyl)-1-(3-(4-tolyl)-[1,2,4]triazolo[4,3-b]pyridazin-6-yl)azetidine-3-carboxamide

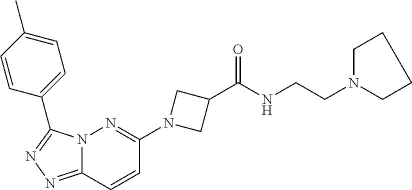 [SMILES: Cc1ccc(-c2nnc3ccc(N4CC(C(=O)NCCN5CCCC5)C4)nn23)cc1]

General procedure F was employed using methyl 1-(3-(4-tolyl)-[1,2,4]triazolo[4,3-b]pyridazin-6-yl)azetidine-3-carboxylate (40 mg, 0.12 mmol, 1 eq.) and 2-(pyrrolidin-1-yl) ethan-1-amine (2 mL). A white solid (29 mg, 58%) was isolated by CombiFlash chromatography (C18, ACN/Water, 1:1). ¹H NMR (400 MHZ, MeOD) δ 8.32 (d, J=8.3 Hz, 2H), 7.98 (d, J=9.9 Hz, 1H), 7.39 (d, J=8.1 Hz, 2H), 6.92 (d, J=9.9 Hz, 1H), 4.41-4.28 (m, 4H), 3.68-3.59 (m, 1H), 3.43 (t, J=6.8 Hz, 2H), 2.69-2.58 (m, 6H), 2.45 (s, 3H), 1.89-1.80 (m, 4H).

### Example 33. N-(2-(Diethylamino)ethyl)-4-(3-(3-tolyl)-[1,2,4]triazolo[4,3-a]pyridin-6-yl)benzamide

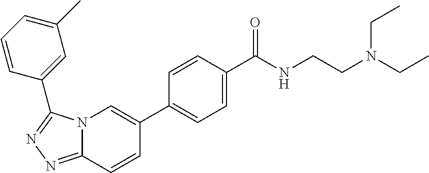 [SMILES: CCN(CC)CCNC(=O)c1ccc(-c2ccc3nnc(-c4cccc(C)c4)n3c2)cc1]

General procedure D4 was employed using 4-(3-(3-tolyl)-[1,2,4]triazolo[4,3-a]pyridin-6-yl)benzoic acid (40 mg, 0.12 mmol, 1 eq.) and N¹,N¹-diethylethane-1,2-diamine (35 μL, 0.24 mmol, 2 eq.). A white solid (20 mg, 39%) was isolated by CombiFlash chromatography (silica gel, MeOH/CH₂Cl₂, 3:17). ¹H NMR (400 MHZ, MeOD) δ 8.63 (t, J=1.4 Hz, 1H), 8.00-7.86 (m, 4H), 7.84-7.78 (m, 2H), 7.77-7.71 (m, 2H), 7.57 (t, J=7.6 Hz, 1H), 7.49 (d, J=7.6 Hz, 1H), 3.55 (t, J=6.9 Hz, 2H), 2.76 (t, J=7.1 Hz, 2H), 2.69 (q, J=7.2 Hz, 4H), 2.51 (s, 3H), 1.13 (t, J=7.2 Hz, 6H).

### Example 34. N-(2-Morpholinoethyl)-4-(3-(3-tolyl)-[1,2,4]triazolo[4,3-a]pyridin-6-yl)benzamide

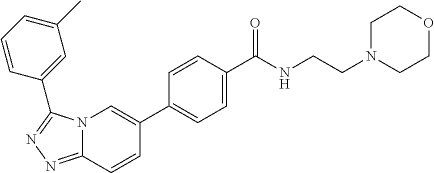 [SMILES: Cc1cccc(-c2nnc3ccc(-c4ccc(C(=O)NCCN5CCOCC5)cc4)cn23)c1]

General procedure D4 was employed using 4-(3-(3-tolyl)-[1,2,4]triazolo[4,3-a]pyridin-6-yl)benzoic acid (40 mg, 0.12 mmol, 1 eq.) and 2-morpholinoethan-1-amine (32 μL, 0.24 mmol, 2 eq.). A white solid (24 mg, 45%) was isolated by CombiFlash chromatography (silica gel, MeOH/CH₂Cl₂, 3:17). ¹H NMR (400 MHZ, CDCl₃) δ 8.35 (s, 1H), 7.88-7.80 (m, 3H), 7.68-7.52 (m, 4H), 7.49 (dd, J=9.6, 1.6 Hz, 1H), 7.43 (t, J=7.6 Hz, 1H), 7.33 (d, J=7.7 Hz, 1H), 6.83 (s, 1H), 3.68 (t, J=4.6 Hz, 4H), 3.53 (q, J=5.5 Hz, 2H), 2.59 (t, J=5.9 Hz, 2H), 2.53-2.37 (m, 7H).

### Example 35. N-(2-(Diisopropylamino)ethyl)-4-(3-(3-tolyl)-[1,2,4]triazolo[4,3-a]pyridin-6-yl)benzamide

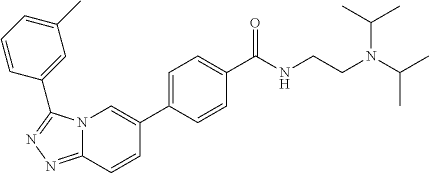 [SMILES: Cc1cccc(-c2nnc3ccc(-c4ccc(C(=O)NCCN(C(C)C)C(C)C)cc4)cn23)c1]

General procedure D4 was employed using 4-(3-(3-tolyl)-[1,2,4]triazolo[4,3-a]pyridin-6-yl)benzoic acid (40 mg, 0.12 mmol, 1 eq.) and N¹,N¹-diisopropylethane-1,2-diamine (44 μL, 0.24 mmol, 2 eq.). A white solid (22 mg, 40%) was isolated by CombiFlash chromatography (silica gel, MeOH/CH₂Cl₂, 3:17). ¹H NMR (400 MHZ, CDCl₃) δ 9.20 (s, 1H), 8.43 (s, 1H), 8.23 (d, J=8.4 Hz, 2H), 7.92 (d, J=9.5 Hz, 1H), 7.71 (s, 1H), 7.68-7.60 (m, 2H), 7.56 (dd, J=9.5, 1.6 Hz, 1H), 7.52 (t, J=7.7 Hz, 2H), 7.40 (d, J=7.8 Hz, 1H), 3.94 (t, J=7.8 Hz, 2H), 3.68-3.58 (m, 2H), 3.24 (dd, J=5.5 Hz, 3.5 Hz, 2H), 2.49 (s, 3H), 1.47 (dd, J=34.9, 6.6 Hz, 12H).

### Example 36. N-Isopentyl-4-(3-(3-tolyl)-[1,2,4]triazolo[4,3-a]pyridin-6-yl)benzamide

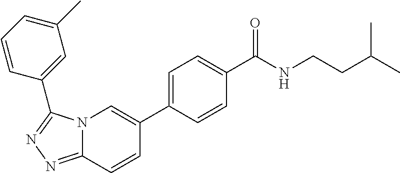 [SMILES: Cc1cccc(-c2nnc3ccc(-c4ccc(C(=O)NCCC(C)C)cc4)cn23)c1]

General procedure D4 was employed using 4-(3-(3-tolyl)-[1,2,4]triazolo[4,3-a]pyridin-6-yl)benzoic acid (40 mg, 0.12 mmol, 1 eq.) and 3-methylbutan-1-amine (28 μL, 0.24 mmol, 2 eq.). A white solid (27 mg, 56%) was isolated by CombiFlash chromatography (silica gel, MeOH/CH₂Cl₂, 3:17). ¹H NMR (400 MHZ, CDCl₃) δ 8.42 (s, 1H), 7.92 (dd, J=9.7, 0.9 Hz, 1H), 7.88 (dt, J=8.4 Hz, 1.9 Hz, 2H), 7.71 (s, 1H), 7.65-7.58 (m, 3H), 7.55 (dd, J=9.5 Hz, 1.7 Hz, 1H), 7.50 (t, J=7.6 Hz, 1H), 7.40 (d, J=7.7 Hz, 1H), 6.09 (s, 1H), 3.51 (dd, J=7.7 Hz, 6.8 Hz, 2H), 2.48 (s, 3H), 1.71 (s, J=6.6 Hz, 1H), 1.56-1.49 (m, 2H), 0.98 (d, J=6.6 Hz, 6H).

### Example 37. N-(2-Morpholinoethyl)-1-(3-(3-tolyl)-[1,2,4]triazolo[4,3-b]pyridazin-6-yl)piperidine-4-carboxamide

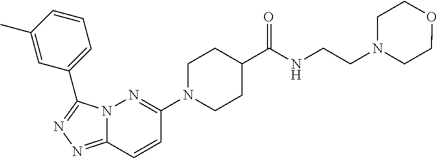 [SMILES: Cc1cccc(-c2nnc3ccc(N4CCC(C(=O)NCCN5CCOCC5)CC4)nn23)c1]

General procedure F was employed using methyl 1-(3-(3-tolyl)-[1,2,4]triazolo[4,3-b]pyridazin-6-yl)piperidine-4-carboxylate (40 mg, 0.11 mmol, 1 eq.) and 2-morpholinoethan-1-amine (2 mL). A white solid (28 mg, 54%) was isolated by CombiFlash chromatography (C18, ACN/Water, 1:1). ¹H NMR (400 MHZ, CDCl₃) δ 8.26-8.15 (m, 2H), 7.84 (d, J=10.1 Hz, 1H), 7.34 (t, J=7.7 Hz, 1H), 7.22 (d, J=7.4 Hz, 1H), 6.90 (d, J=10.1 Hz, 1H), 6.13 (d, J=5.1 Hz, 1H), 4.17 (dt, J=13.6, 3.8 Hz, 2H), 3.64 (t, J=4.7 Hz, 4H), 3.31 (q, J=5.6 Hz, 2H), 3.03 (td, J=13.0 Hz, 3.0 Hz, 2H), 2.47-2.28 (m, 12H), 1.92-1.72 (m, 2H).

### Example 38. N-(2-(Diisopropylamino)ethyl)-1-(3-(3-tolyl)-[1,2,4]triazolo[4,3-b]pyridazin-6-yl)piperidine-4-carboxamide

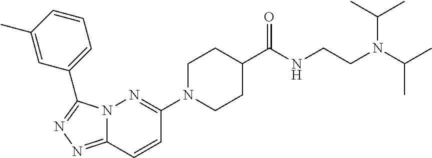 [SMILES: Cc1cccc(-c2nnc3ccc(N4CCC(C(=O)NCCN(C(C)C)C(C)C)CC4)nn23)c1]

General procedure F was employed using methyl 1-(3-(3-tolyl)-[1,2,4]triazolo[4,3-b]pyridazin-6-yl)piperidine-4-carboxylate (40 mg, 0.11 mmol, 1 eq.) and N¹,N¹-diisopropylethane-1,2-diamine (1 mL). A white solid (34 mg, 65%) was isolated by CombiFlash chromatography (C18, ACN/Water, 1:1). ¹H NMR (400 MHZ, MeOD) δ 8.25 (s, 1H), 8.21 (d, J=7.9 Hz, 1H), 7.99 (d, J=10.2 Hz, 1H), 7.50-7.42 (m, 2H), 7.38 (d, J=7.6 Hz, 1H), 4.39 (d, J=13.6 Hz, 2H), 3.23-3.01 (m, 6H), 2.62-2.50 (m, 3H), 2.48 (s, 3H), 1.99-1.78 (m, 4H), 1.06 (d, J=6.5 Hz, 12H).

### Example 39. N-(2-(Diethylamino)ethyl)-4-(3-(4-fluorophenyl)-[1,2,4]triazolo[4,3-a]pyridin-6-yl)-3-methylbenzamide

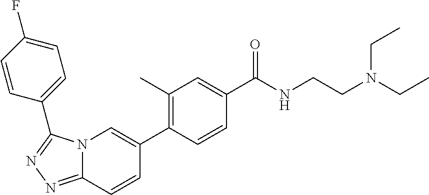 [SMILES: CCN(CC)CCNC(=O)c1ccc(-c2ccc3nnc(-c4ccc(F)cc4)n3c2)c(C)c1]

General procedure D4 was employed using 4-(3-(4-fluorophenyl)-[1,2,4]triazolo[4,3-a]pyridin-6-yl)-3-methylbenzoic acid (40 mg, 0.12 mmol, 1 eq.) and N¹,N¹-diethylethane-1,2-diamine (32 μL, 0.24 mmol, 2 eq.). A white solid (41 mg, 80%) was isolated by CombiFlash chromatography (silica gel, MeOH/CH₂Cl₂, 3:17). ¹H NMR (400 MHZ, CDCl₃) δ 8.13 (t, J=1.3 Hz, 1H), 7.89 (dd, J=9.5, 1.1 Hz, 1H), 7.87-7.81 (m, 2H), 7.78 (d, J=1.8 Hz, 1H), 7.66 (dd, J=7.7 Hz, 2.2 Hz, 1H), 7.33-7.27 (m, 4H), 7.11 (s, 1H), 3.52 (q, J=5.5 Hz, 2H), 2.70 (t, J=5.9 Hz, 2H), 2.61 (q, J=7.1 Hz, 4H), 2.36 (s, 3H), 1.07 (t, J=7.1 Hz, 6H).

### Example 40. Azepan-1-yl(4-(3-(4-fluorophenyl)-[1,2,4]triazolo[4,3-a]pyridin-6-yl)-3-methylphenyl)methanone

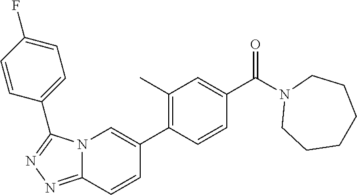 [SMILES: Cc1cc(C(=O)N2CCCCCC2)ccc1-c1ccc2nnc(-c3ccc(F)cc3)n2c1]

General procedure D4 was employed using 4-(3-(4-fluorophenyl)-[1,2,4]triazolo[4,3-a]pyridin-6-yl)-3-methylbenzoic acid (38 mg, 0.11 mmol, 1 eq.) and azepane (25 μL, 0.22 mmol, 2 eq.). A white solid (42 mg, 92%) was isolated by CombiFlash chromatography (silica gel, MeOH/CH₂Cl₂, 3:17). ¹H NMR (400 MHz, CDCl₃) δ 8.12 (t, J=1.3 Hz, 1H), 7.89 (dd, J=9.4, 1.1 Hz, 1H), 7.87-7.80 (m, 2H), 7.35-7.26 (m, 6H), 3.70 (t, J=5.3 Hz, 2H), 3.43 (t, J=5.7 Hz, 2H), 2.32 (s, 3H), 1.90-1.82 (m, 2H), 1.75-1.61 (m, 6H).

### Example 41. (4-(3-(4-Fluorophenyl)-[1,2,4]triazolo[4,3-a]pyridin-6-yl)-3-methylphenyl)-(piperidin-1-yl)methanone

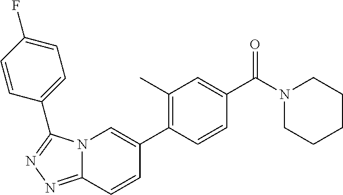 [SMILES: Cc1cc(C(=O)N2CCCCC2)ccc1-c1ccc2nnc(-c3ccc(F)cc3)n2c1]

General procedure D4 was employed using 4-(3-(4-fluorophenyl)-[1,2,4]triazolo[4,3-a]pyridin-6-yl)-3-methylbenzoic acid (40 mg, 0.12 mmol, 1 eq.) and piperidine (23 μL, 0.23 mmol, 2 eq.). A white solid (44 mg, 92%) was isolated by CombiFlash chromatography (silica gel, MeOH/CH₂Cl₂, 3:17). ¹H NMR (400 MHZ, CDCl₃) δ 8.12 (t, J=1.3 Hz, 1H), 7.88 (dd, J=9.4, 1.1 Hz, 2H), 7.88-7.79 (m, 3H), 7.36 (d, J=1.5 Hz, 1H), 7.33-7.26 (m, 3H), 3.77-3.68 (m, 2H), 3.45-3.33 (m, 2H), 2.32 (s, 3H), 1.75-1.65 (m, 4H), 1.57-1.50 (m, 2H).

### Example 42. N-(2-(Diethylamino)ethyl)-1-(3-(naphthalen-1-yl)-[1,2,4]triazolo[4,3-b]pyridazin-6-yl)azetidine-3-carboxamide

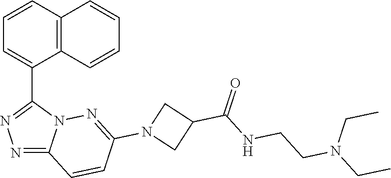 [SMILES: CCN(CC)CCNC(=O)C1CN(c2ccc3nnc(-c4cccc5ccccc45)n3n2)C1]

General procedure F was employed using methyl 1-[3-(1-naphthyl)-1,2,3a,4-tetraaza-5-indenyl]-3-azetidinecarboxylate (40 mg, 0.11 mmol, 1 eq.) and N¹,N¹-diethylethane-1,2-diamine (2 mL). A white solid (26 mg, 53%) was isolated by CombiFlash chromatography (C18, ACN/Water, 1:1). ¹H NMR (400 MHZ, CDCl₃) δ 8.34-8.27 (m, 1H), 8.05 (dd, J=7.2, 1.2 Hz, 1H), 8.00 (d, J=8.3 Hz, 1H), 7.97 (d, J=9.8 Hz, 1H), 7.94-7.88 (m, 1H), 7.60 (dd, J=8.2, 7.2 Hz, 1H), 7.56-7.48 (m, 2H), 6.58 (d, J=9.9 Hz, 1H), 4.26-4.15 (m, 4H), 3.59-3.44 (m, 3H), 2.96-2.83 (m, 6H), 1.28-1.17 (m, 6H).

### Example 43. N-2-(Diethylamino)ethyl-1-[3-(4-chlorophenyl)-1,2,3a,4-tetraaza-5-indenyl]-3-azetidinecarboxamide

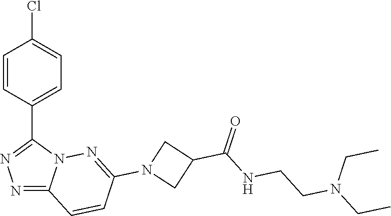 [SMILES: CCN(CC)CCNC(=O)C1CN(c2ccc3nnc(-c4ccc(Cl)cc4)n3n2)C1]

General procedure F was employed using methyl 1-[3-(4-chlorophenyl)-1,2,3a,4-tetraaza-5-indenyl]-3-azetidinecarboxylate (40 mg, 0.11 mmol, 1 eq.) and N¹,N¹-diethylethane-1,2-diamine (2 mL). A white solid (29 mg, 58%) was isolated by CombiFlash chromatography (C18, ACN/Water, 1:1). ¹H NMR (400 MHZ, CDCl₃) δ 8.81 (t, J=6.6 Hz, 1H), 8.48 (dt, J=8.7, 2.4 Hz, 2H), 8.04 (d, J=9.8 Hz, 1H), 7.50 (dt, J=8.7, 1.9 Hz, 2H), 6.65 (d, J=9.9 Hz, 1H), 4.40 (d, J=7.4 Hz, 4H), 3.82-3.70 (m, 3H), 3.33-3.08 (m, 6H), 1.45 (t, J=7.3 Hz, 6H).

### Example 44. N-2-(Diethylamino)ethyl-1-[3-(3,4-dichlorophenyl)-1,2,3a,4-tetraaza-5-indenyl]-3-azetidinecarboxamide

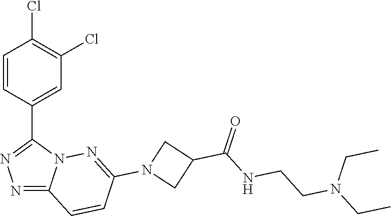 [SMILES: CCN(CC)CCNC(=O)C1CN(c2ccc3nnc(-c4ccc(Cl)c(Cl)c4)n3n2)C1]

General procedure F was employed methyl 1-[3-(3,4-dichlorophenyl)-1,2,3a,4-tetraaza-5-indenyl]-3-azetidinecarboxylate (30 mg, 0.080 mmol, 1 eq.) and N¹,N¹-diethylethane-1,2-diamine (2 mL). A white solid (16 mg, 44%) was isolated by CombiFlash chromatography (C18, ACN/Water, 1:1). ¹H NMR (400 MHZ, CDCl₃) δ 8.83-8.73 (m, 2H), 8.40 (dd, J=8.4, 2.1 Hz, 1H), 7.93 (d, J=9.8 Hz, 1H), 7.58 (d, J=8.5 Hz, 1H), 6.58 (d, J=9.8 Hz, 1H), 4.39 (d, J=7.2 Hz, 4H), 3.81-3.69 (m, 3H), 3.30-3.10 (m, 6H), 1.45 (t, J=7.3 Hz, 6H).

### Example 45. N-2-(Diethylamino)ethyl-1-[3-(2-tolyl)-1,2,3a,4-tetraaza-5-indenyl]-3-azetidinecarboxamide

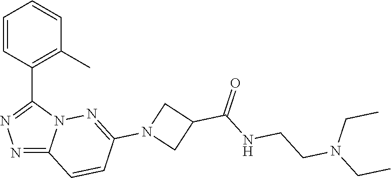 [SMILES: CCN(CC)CCNC(=O)C1CN(c2ccc3nnc(-c4ccccc4C)n3n2)C1]

General procedure F was employed methyl 1-[3-(2-tolyl)-1,2,3a,4-tetraaza-5-indenyl]-3-azetidinecarboxylate (30 mg, 0.093 mmol, 1 eq.) and N¹,N¹-diethylethane-1,2-diamine (2 mL). A white solid (17 mg, 45%) was isolated by CombiFlash chromatography (C18, ACN/Water, 1:1). ¹H NMR (400 MHZ, CDCl₃) δ 7.91 (d, J=9.8 Hz, 1H), 7.70 (dd, J=7.7, 1.4 Hz, 1H), 7.43-7.27 (m, 3H), 6.54 (d, J=9.8 Hz, 1H), 4.31-4.17 (m, 4H), 3.69-3.48 (m, 3H), 3.09-2.82 (m, 6H), 2.41 (s, 3H), 1.28 (t, J=11.9 Hz, 6H).

### Example 46. N-2-(Diethylamino)ethyl-1-(3-cyclohexyl-1,2,3a,4-tetraaza-5-indenyl)-3-azetidinecarboxamide

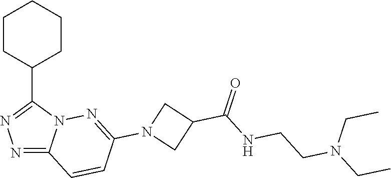 [SMILES: CCN(CC)CCNC(=O)C1CN(c2ccc3nnc(C4CCCCC4)n3n2)C1]

General procedure F was employed methyl 1-(3-cyclohexyl-1,2,3a,4-tetraaza-5-indenyl)-3-azetidinecarboxylate (40 mg, 0.13 mmol, 1 eq.) and N¹,N¹-diethylethane-1,2-diamine (2 mL). A white solid (32 mg, 63%) was isolated by CombiFlash chromatography (C18, ACN/Water, 1:1). ¹H NMR (400 MHZ, CDCl₃) δ 8.82 (t, J=5.5 Hz, 1H), 8.02 (d, J=9.8 Hz, 1H), 6.65 (d, J=9.8 Hz, 1H), 4.35 (d, J=7.3 Hz, 4H), 3.80-3.63 (m, 3H), 3.34-3.08 (m, 8H), 2.14-2.02 (m, 2H), 1.95-1.70 (m, 5H), 1.45 (t, J=7.3 Hz, 6H), 1.42-1.30 (m, 2H).

### Example 47. 2-Methoxy-N-(2-(pyrrolidin-1-yl)ethyl)-4-(3-(3-(trifluoromethyl)phenyl)imidazo[1,5-a]pyridin-6-yl)benzamide

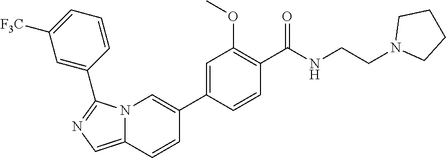 [SMILES: COc1cc(-c2ccc3cnc(-c4cccc(C(F)(F)F)c4)n3c2)ccc1C(=O)NCCN1CCCC1]

General procedure D1 was employed using 2-methoxy-4-(3-(3-(trifluoromethyl)phenyl)imidazo[1,5-a]pyridin-6-yl)benzoic acid (50 mg, 0.12 mmol, 1 eq.) and 2-(pyrrolidin-1-yl) ethan-1-amine (30 μL, 0.24 mmol, 2 eq.). A yellow solid (39 mg, 63%) was isolated by CombiFlash chromatography (silica gel, MeOH/CH₂Cl₂, 3:17). ¹H NMR (400 MHZ, CDCl₃) δ 8.43 (s, 1H), 8.42-8.36 (m, 1H), 8.29 (d, J=8.1 Hz, 1H), 8.16 (s, 1H), 8.04 (d, J=7.3 Hz, 1H), 7.73-7.61 (m, 4H), 7.23 (d, J=1.7 Hz, 1H), 7.07 (d, J=9.7 Hz, 2H), 4.01 (s, 3H), 3.61 (q, J=5.8 Hz, 2H), 2.77-2.70 (m, 2H), 2.66-2.55 (m, 4H), 1.88-1.77 (m, 4H).

### Example 48. N-(2-(Diethylamino)ethyl)-4-(3-(3-tolyl)imidazo[1,5-a]pyridin-6-yl)benzamide

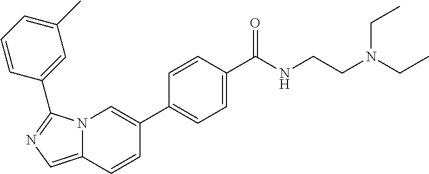 [SMILES: CCN(CC)CCNC(=O)c1ccc(-c2ccc3cnc(-c4cccc(C)c4)n3c2)cc1]

General procedure D1 was employed using 4-(3-(3-tolyl)imidazo[1,5-a]pyridin-6-yl)benzoic acid (40 mg, 0.12 mmol, 1 eq.) and N¹,N¹-diethylethane-1,2-diamine (34 μL, 0.24 mmol, 2 eq.). A yellow solid (32 mg, 62%) was isolated by CombiFlash chromatography (silica gel, MeOH/CH₂Cl₂, 3:17). ¹H NMR (400 MHZ, CDCl₃) δ 9.17 (t, J=5.8 Hz, 1H), 8.45 (s, J=1.3 Hz, 1H), 8.26 (dt, J=8.3, 2.0 Hz, 2H), 7.87 (s, 1H), 7.77-7.67 (m, 3H), 7.63 (dt, J=8.3, 1.9 Hz, 2H), 7.55 (t, J=7.7 Hz, 1H), 7.42 (d, J=7.8 Hz, 1H), 7.30 (d, J=9.6 Hz, 1H), 3.92 (q, J=5.5 Hz, 2H), 3.29 (q, J=4.8 Hz, 2H), 3.21 (dtd, J=16.4, 10.1, 6.0 Hz, 4H), 2.50 (s, 3H), 1.45 (t, J=7.3 Hz, 6H).

### Example 49. N-(2-(Diisopropylamino)ethyl)-3-methyl-4-(3-(3-tolyl)imidazo[1,5-a]pyridin-6-yl)benzamide

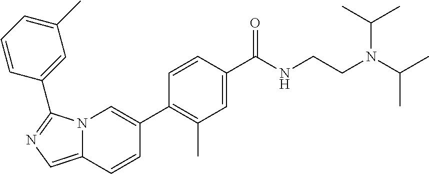 [SMILES: Cc1cccc(-c2ncc3ccc(-c4ccc(C(=O)NCCN(C(C)C)C(C)C)cc4C)cn23)c1]

General procedure D2 was employed using 3-methyl-4-(3-(3-methylphenyl)imidazo[1,5-a]pyridin-6-yl)benzoic acid (40 mg, 0.12 mmol, 1 eq.) and N¹,N¹-diisopropylethane-1,2-diamine (41 μL, 0.23 mmol, 2 eq.). A yellow solid (23 mg, 42%) was isolated by CombiFlash chromatography (C18, ACN, 100%). ¹H NMR (400 MHZ, CDCl₃) δ 8.18 (q, J=1.2 Hz, 1H), 7.74 (s, 1H), 7.65-7.55 (m, 4H), 7.53 (dd, J=9.2, 1.2 Hz, 1H), 7.39 (t, J=7.7 Hz, 1H), 7.30 (d, J=7.9 Hz, 1H), 7.24 (d, J=7.5 Hz, 1H), 7.00 (t, J=5.0 Hz, 1H), 6.73 (dd, J=9.3, 1.3 Hz, 1H), 3.44 (q, J=5.5 Hz, 2H), 3.08 (p, J=6.6 Hz, 2H), 2.72 (t, J=6.0 Hz, 2H), 2.43 (s, 3H), 2.38 (s, 3H), 1.06 (d, J=6.6 Hz, 12H).

### Example 50. 3-Methyl-N-(2-(pyrrolidin-1-yl)ethyl)-4-(3-(3-tolyl)imidazo[1,5-a]pyridin-6-yl)benzamide

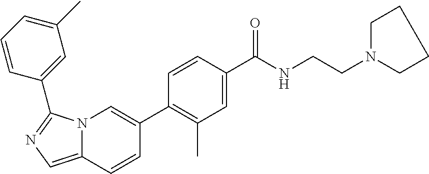 [SMILES: Cc1cccc(-c2ncc3ccc(-c4ccc(C(=O)NCCN5CCCC5)cc4C)cn23)c1]

General procedure D1 was employed using 3-methyl-4-(3-(3-methylphenyl)imidazo[1,5-a]pyridin-6-yl)benzoic acid (40 mg, 0.12 mmol, 1 eq.) and 2-(pyrrolidin-1-yl) ethan-1-amine (30 μL, 0.23 mmol, 2 eq.). A yellow solid (23 mg, 45%) was isolated by CombiFlash chromatography (C18, ACN, 100%). ¹H NMR (400 MHZ, CDCl₃) δ 8.18 (q, J=1.2 Hz, 1H), 7.75 (s, 1H), 7.66-7.59 (m, 3H), 7.57 (d, J=7.8 Hz, 1H), 7.53 (dd, J=9.2, 1.1 Hz, 1H), 7.39 (t, J=7.6 Hz, 1H), 7.31 (d, J=7.9 Hz, 1H), 7.24 (d, J=7.6 Hz, 1H), 6.95 (s, 1H), 6.72 (dd, J=9.3, 1.3 Hz, 1H), 3.58 (q, J=5.6 Hz, 2H), 2.74 (t, J=6.0 Hz, 2H), 2.60 (t, J=5.9 Hz, 4H), 2.43 (s, 3H), 2.39 (s, 3H), 1.81 (p, J=3.0 Hz, 4H).

### Example 51. N-(2-(Diethylamino)ethyl)-2-methyl-4-(3-(3-tolyl)imidazo[1,5-a]pyridin-6-yl)benzamide

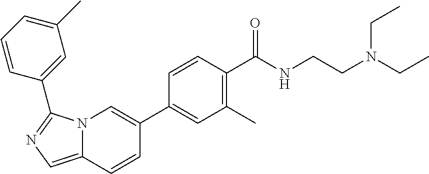 [SMILES: CCN(CC)CCNC(=O)c1ccc(-c2ccc3cnc(-c4cccc(C)c4)n3c2)cc1C]

General procedure D1 was employed using 2-methyl-4-(3-(3-tolyl)imidazo[1,5-a]pyridin-6-yl)benzoic acid (30 mg, 0.088 mmol, 1 eq.) and N¹,N¹-diethylethane-1,2-diamine (25 μL, 0.18 mmol, 2 eq.). A yellow solid (22 mg, 57%) was isolated by CombiFlash chromatography (silica gel, MeOH/CH₂Cl₂, 3:17). ¹H NMR (400 MHZ, CDCl₃) δ 8.57 (t, J=6.1 Hz, 1H), 8.40 (d, J=1.4 Hz, 1H), 7.80 (d, J=8.0 Hz, 1H), 7.72-7.59 (m, 4H), 7.49 (t, J=7.7 Hz, 1H), 7.41 (dd, J=8.0, 1.9 Hz, 1H), 7.37-7.31 (m, 2H), 7.11 (d, J=9.5 Hz, 1H), 3.90 (q, J=5.5 Hz, 2H), 3.29 (q, J=5.3 Hz, 2H), 3.25-3.16 (m, 4H), 2.57 (s, 3H), 2.48 (s, 3H), 1.47 (t, J=7.3 Hz, 6H).

### Example 52. N-(2-(diethylamino)ethyl)-2-methoxy-4-(3-(3-tolyl)imidazo[1,5-a]pyridin-6-yl)benzamide

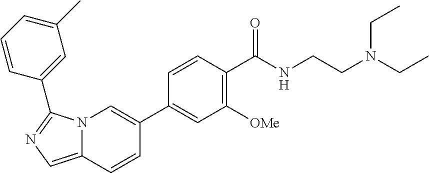 [SMILES: CCN(CC)CCNC(=O)c1ccc(-c2ccc3cnc(-c4cccc(C)c4)n3c2)cc1OC]

General procedure D1 was employed using 2-methoxy-4-(3-(3-tolyl)imidazo[1,5-a]pyridin-6-yl)benzoic acid (50 mg, 0.14 mmol, 1 eq.) and N¹,N¹-diethylethane-1,2-diamine (39 μL, 0.28 mmol, 2 eq.). A yellow solid (42 mg, 66%) was isolated by CombiFlash chromatography (silica gel, MeOH/CH₂Cl₂, 3:17). ¹H NMR (400 MHZ, CDCl₃) δ 8.48-8.39 (m, 2H), 8.29 (d, J=8.1 Hz, 1H), 7.67 (s, 1H), 7.64-7.55 (m, 3H), 7.44 (t, J=7.6 Hz, 1H), 7.29 (d, J=7.5 Hz, 1H), 7.23 (dd, J=8.1, 1.6 Hz, 1H), 7.07 (d, J=1.6 Hz, 1H), 7.00 (dd, J=9.3, 1.4 Hz, 1H), 4.01 (s, 3H), 3.54 (q, J=5.7 Hz, 2H), 2.65 (t, J=6.1 Hz, 2H), 2.59 (q, J=7.1 Hz, 4H), 2.45 (s, 3H), 1.06 (t, J=7.1 Hz, 6H).

### Example 53. N-(2-(Diisopropylamino)ethyl)-2-methoxy-4-(3-(3-tolyl)imidazo[1,5-a]pyridin-6-yl)benzamide

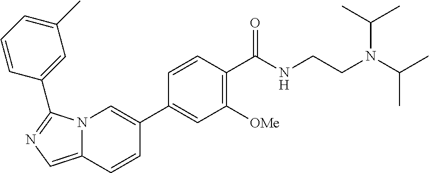 [SMILES: COc1cc(-c2ccc3cnc(-c4cccc(C)c4)n3c2)ccc1C(=O)NCCN(C(C)C)C(C)C]

General procedure D2 was employed using 2-methoxy-4-(3-(3-tolyl)imidazo[1,5-a]pyridin-6-yl)benzoic acid (30 mg, 0.084 mmol, 1 eq.) and N¹,N¹-diisopropylethane-1,2-diamine (30 μL, 0.17 mmol, 2 eq.). A yellow solid (31 mg, 76%) was isolated by CombiFlash chromatography (C18, ACN, 100%). HRMS (FTMS+pESI): m/z calcd for C₃₀H₃₆N₄O₂ [M+H]⁺ 485.2911, found 485.2890.

### Example 54. 2-Methoxy-N-(2-(pyrrolidin-1-yl)ethyl)-4-(3-(3-tolyl)imidazo[1,5-a]pyridin-6-yl)benzamide

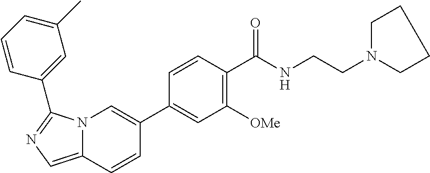 [SMILES: COc1cc(-c2ccc3cnc(-c4cccc(C)c4)n3c2)ccc1C(=O)NCCN1CCCC1]

General procedure D2 was employed using 2-methoxy-4-(3-(3-tolyl)imidazo[1,5-a]pyridin-6-yl)benzoic acid (30 mg, 0.084 mmol, 1 eq.) and 2-(pyrrolidin-1-yl) ethan-1-amine (21 μL, 0.17 mmol, 2 eq.). A yellow solid (17 mg, 45%) was isolated by CombiFlash chromatography (C18, ACN, 100%). ¹H NMR (400 MHZ, CDCl₃) δ 8.42-8.35 (m, 1H), 8.39 (s, 1H), 8.27 (d, J=8.1 Hz, 1H), 7.68-7.55 (m, 4H), 7.44 (t, J=7.6 Hz, 1H), 7.29 (d, J=10.7 Hz, 1H), 7.23 (dd, J=8.1, 1.6 Hz, 1H), 7.06 (d, J=1.6 Hz, 1H), 7.00 (dd, J=9.4, 1.4 Hz, 1H), 4.00 (s, 3H), 3.60 (q, J=5.9 Hz, 2H), 2.72 (t, J=6.3 Hz, 2H), 2.59 (t, J=6.5 Hz, 4H), 2.45 (s, 3H), 1.81 (p, J=3.4 Hz, 4H).

### Example 55. N-(2-(Diethylamino)ethyl)-2-isopropyl-4-(3-(3-tolyl)imidazo[1,5-a]pyridin-6-yl)benzamide

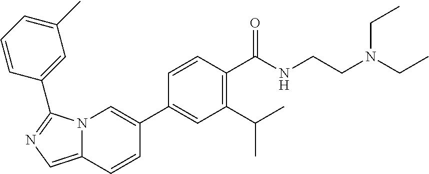 [SMILES: CCN(CC)CCNC(=O)c1ccc(-c2ccc3cnc(-c4cccc(C)c4)n3c2)cc1C(C)C]

General procedure D2 was employed using 2-isopropyl-4-(3-(3-tolyl)imidazo[1,5-a]pyridin-6-yl)benzoic acid (40 mg, 0.11 mmol, 1 eq.) and N¹,N¹-diethylethane-1,2-diamine (31 μL, 0.22 mmol, 2 eq.). A yellow solid (21 mg, 41%) was isolated by CombiFlash chromatography (C18, ACN, 100%). ¹H NMR (400 MHZ, CDCl₃) δ 8.42 (s, 1H), 7.67 (s, 1H), 7.65-7.54 (m, 3H), 7.53-7.48 (m, 2H), 7.43 (t, J=7.6 Hz, 1H), 7.36 (dd, J=7.9, 1.8 Hz, 1H), 7.28 (s, 1H), 6.99 (dd, J=9.4, 1.5 Hz, 1H), 3.81-3.63 (m, 2H), 3.46 (p, J=6.9 Hz, 1H), 3.22-2.69 (br, 6H), 2.45 (s, 3H), 1.30 (d, J=6.9 Hz, 6H), 1.28-1.15 (m, 6H).

### Example 56. 2-Chloro-N-(2-(diethylamino)ethyl)-4-(3-(3-tolyl)imidazo[1,5-a]pyridin-6-yl)benzamide

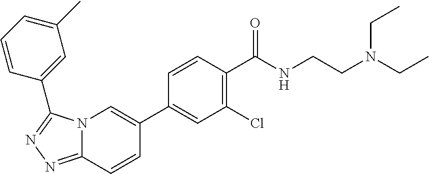 [SMILES: CCN(CC)CCNC(=O)c1ccc(-c2ccc3nnc(-c4cccc(C)c4)n3c2)cc1Cl]

General procedure D2 was employed using 2-chloro-4-(3-(3-tolyl)imidazo[1,5-a]pyridin-6-yl)benzoic acid (20 mg, 0.055 mmol, 1 eq.) and N¹,N¹-diethylethane-1,2-diamine (16 μL, 0.11 mmol, 2 eq.). A yellow solid (19 mg, 75%) was isolated by CombiFlash chromatography (C18, ACN, 100%). ¹H NMR (400 MHZ, CDCl₃) δ 8.40 (q, J=1.1 Hz, 1H), 7.76 (d, J=8.0 Hz, 1H), 7.65 (s, 1H), 7.62-7.53 (m, 4H), 7.50-7.41 (m, 2H), 7.29 (d, J=7.5 Hz, 1H), 6.95 (dd, J=9.3, 1.5 Hz, 1H), 3.87-3.68 (m, 2H), 3.26-2.66 (m, 6H), 2.46 (s, 3H), 1.39-1.26 (m, 6H).

### Example 57. N-(2-(Diethylamino)ethyl)-5-(3-(3-tolyl)imidazo[1,5-a]pyridin-6-yl)thiophene-2-carboxamide

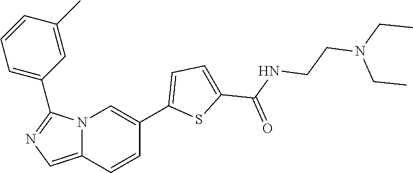 [SMILES: CCN(CC)CCNC(=O)c1ccc(-c2ccc3cnc(-c4cccc(C)c4)n3c2)s1]

General procedure D3 was employed using 5-(3-(3-tolyl)imidazo[1,5-a]pyridin-6-yl)thiophene-2-carboxylic acid (25 mg, 0.075 mmol, 1 eq.) and N¹,N¹-diethylethane-1,2-diamine (21 μL, 0.15 mmol, 2 eq.). A yellow solid (17 mg, 53%) was isolated by prepHPLC (ACN/H₂O, 9:1). ¹H NMR (400 MHZ, CDCl₃) δ 8.98-8.79 (br, 1H), 8.52 (d, J=1.4 Hz, 1H), 7.98 (d, J=3.9 Hz, 1H), 7.65 (s, 1H), 7.62-7.55 (m, 2H), 7.53 (dd, J=9.4, 1.1 Hz, 1H), 7.44 (t, J=7.7 Hz, 1H), 7.29 (d, J=7.5 Hz, 1H), 7.23 (d, J=3.9 Hz, 1H), 6.98 (dd, J=9.4, 1.5 Hz, 1H), 3.84 (q, J=3.8 Hz, 2H), 3.28-3.04 (m, 6H), 2.46 (s, 3H), 1.42 (t, J=7.2 Hz, 6H). ¹³C NMR (101 MHZ, MeOD) δ 162.44, 144.40, 139.21, 139.12, 138.05, 130.56, 129.85, 129.12, 128.85, 128.57, 124.86, 124.74, 121.03, 119.95, 118.92, 118.78, 117.56, 51.14, 46.80, 36.83, 20.10, 10.18.

### Example 58. N-(2-(Diethylamino)ethyl)-5-(3-(3-tolyl)imidazo[1,5-a]pyridin-6-yl)-1H-pyrrole-2-carboxamide

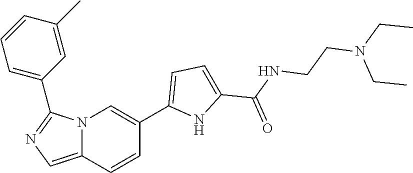 [SMILES: CCN(CC)CCNC(=O)c1ccc(-c2ccc3cnc(-c4cccc(C)c4)n3c2)[nH]1]

General procedure D3 was employed using 5-(3-(3-tolyl)imidazo[1,5-a]pyridin-6-yl)-1H-pyrrole-2-carboxylic acid (12 mg, 0.038 mmol, 1 eq.) and N¹,N¹-diethylethane-1,2-diamine (11 μL, 0.075 mmol, 2 eq.). A yellow solid (12 mg, 76%) was isolated by prepHPLC (ACN/H₂O, 9:1). ¹H NMR (400 MHZ, CDCl₃) δ 8.82-8.67 (br, 1H), 8.54 (s, 1H), 7.70-7.66 (m, 2H), 7.56-7.50 (m, 2H), 7.45 (t, J=7.9 Hz, 1H), 7.14 (d, J=9.4 Hz, 1H), 7.08 (s, 1H), 6.47 (dd, J=3.9, 2.5 Hz, 1H), 3.82 (q, J=5.6 Hz, 2H), 3.28-3.10 (m, 6H), 2.46 (s, 3H), 1.43 (t, J=7.4 Hz, 6H).

### Example 59. N-(2-(Diethylamino)ethyl)-5-(3-(3-tolyl)imidazo[1,5-a]pyridin-6-yl) furan-3-carboxamide

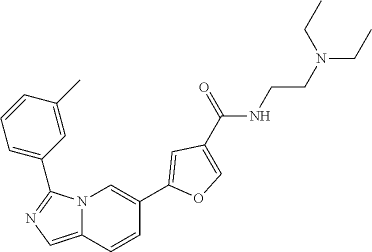 [SMILES: CCN(CC)CCNC(=O)c1coc(-c2ccc3cnc(-c4cccc(C)c4)n3c2)c1]

General procedure D3 was employed using 5-(3-(3-tolyl)imidazo[1,5-a]pyridin-6-yl) furan-3-carboxylic acid (20 mg, 0.063 mmol, 1 eq.) and N¹,N¹-diethylethane-1,2-diamine (18 μL, 0.13 mmol, 2 eq.). A yellow solid (17 mg, 65%) was isolated by prepHPLC (ACN/H₂O, 9:1). ¹H NMR (400 MHZ, CDCl₃) δ 8.80-8.60 (br, 1H), 8.62 (s, 1H), 8.23 (s, 1H), 7.66 (s, 1H), 7.62 (d, J=7.7 Hz, 1H), 7.55 (s, 1H), 7.51 (d, J=9.6 Hz, 1H), 7.46 (t, J=7.6 Hz, 1H), 7.30 (d, J=8.3 Hz, 1H), 7.21 (s, 1H), 7.00 (d, J=9.1 Hz, 1H), 3.85-3.74 (m, 2H), 3.24-3.06 (m, 6H), 2.47 (s, 3H), 1.41 (t, J=7.8 Hz, 6H).

### Example 60. N-(2-(Diethylamino)ethyl)-5-(3-(3-tolyl)imidazo[1,5-a]pyridin-6-yl)thiophene-3-carboxamide

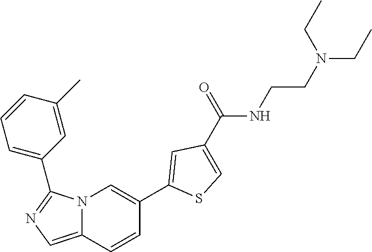 [SMILES: CCN(CC)CCNC(=O)c1csc(-c2ccc3cnc(-c4cccc(C)c4)n3c2)c1]

General procedure D3 was employed using 5-(3-(3-tolyl)imidazo[1,5-a]pyridin-6-yl)thiophene-3-carboxylic acid (20 mg, 0.060 mmol, 1 eq.) and N¹,N¹-diethylethane-1,2-diamine (17 μL, 0.12 mmol, 2 eq.). A yellow solid (16 mg, 62%) was isolated by prepHPLC (ACN/H₂O, 9:1). ¹H NMR (400 MHZ, CDCl₃) δ 8.84-8.57 (br, 1H), 8.50 (d, J=1.2 Hz, 1H), 8.21 (s, 1H), 7.86 (s, 1H), 7.66 (s, 1H), 7.62 (d, J=7.7 Hz, 1H), 7.56 (s, 1H), 7.53 (dd, J=9.5, 1.1 Hz, 1H), 7.46 (t, J=7.7 Hz, 1H), 7.29 (d, J=7.6 Hz, 1H), 7.04 (dd, J=9.4, 1.5 Hz, 1H), 3.80 (q, J=4.5 Hz, 2H), 3.22-3.01 (m, 6H), 2.46 (s, 3H), 1.39 (t, J=6.0 Hz, 6H).

### Example 61. N-(2-(Pyrrolidin-1-yl)ethyl)-5-(3-(3-tolyl)imidazo[1,5-a]pyridin-6-yl)thiophene-3-carboxamide

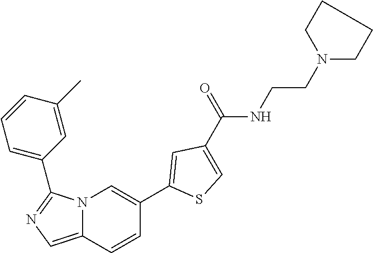 [SMILES: Cc1cccc(-c2ncc3ccc(-c4cc(C(=O)NCCN5CCCC5)cs4)cn23)c1]

General procedure D3 was employed using 5-(3-(3-tolyl)imidazo[1,5-a]pyridin-6-yl)thiophene-3-carboxylic acid (20 mg, 0.060 mmol, 1 eq.) and 2-(pyrrolidin-1-yl) ethan-1-amine (15 μL, 0.12 mmol, 2 eq.). A yellow solid (4 mg, 15%) was isolated by prepHPLC (ACN/H₂O, 9:1). ¹H NMR (400 MHZ, CDCl₃) δ 8.50 (d, J=1.2 Hz, 1H), 8.22 (s, 1H), 7.88 (s, 1H), 7.66 (s, 1H), 7.62 (d, J=7.7 Hz, 1H), 7.57-7.50 (m, 2H), 7.46 (t, J=7.6 Hz, 1H), 7.28 (d, J=6.4 Hz, 1H), 7.04 (dd, J=9.3, 1.5 Hz, 1H), 3.81 (q, J=5.5 Hz, 2H), 3.25-3.16 (m, 2H), 2.46 (s, 3H), 2.25-2.08 (br, 4H), 1.28-1.21 (m, 4H).

### Example 62. N-(2-(Diethylamino)ethyl)-5-(3-(3-tolyl)imidazo[1,5-a]pyridin-6-yl) picolinamide

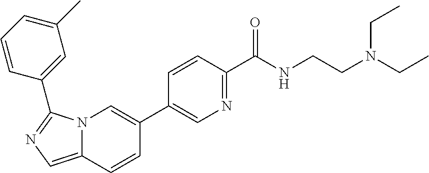 [SMILES: CCN(CC)CCNC(=O)c1ccc(-c2ccc3cnc(-c4cccc(C)c4)n3c2)cn1]

General procedure D2 was employed using 5-(3-(3-tolyl)imidazo[1,5-a]pyridin-6-yl) picolinic acid (20 mg, 0.061 mmol, 1 eq.) and N¹,N¹-diethylethane-1,2-diamine (17 μL, 0.12 mmol, 2 eq.). A yellow solid (18 mg, 69%) was isolated by CombiFlash chromatography (C18, ACN, 100%). ¹H NMR (400 MHZ, CDCl₃) δ 8.77 (s, 1H), 8.46 (s, 1H), 8.25 (d, J=8.1 Hz, 1H), 7.98 (dd, J=8.1, 2.3 Hz, 1H), 7.68-7.55 (m, 4H), 7.45 (t, J=7.6 Hz, 1H), 7.29 (d, J=7.5 Hz, 1H), 6.98 (dd, J=9.4, 1.4 Hz, 1H), 3.73-3.52 (m, 2H), 3.06-2.54 (m, 6H), 2.46 (s, 3H), 1.20-1.00 (m, 6H).

### Example 63. 4-(3-(3-Chlorophenyl)imidazo[1,5-a]pyridin-6-yl)-N-(2-(diethylamino)ethyl)-3-methylbenzamide

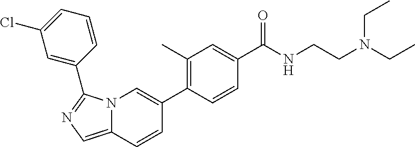 [SMILES: CCN(CC)CCNC(=O)c1ccc(-c2ccc3cnc(-c4cccc(Cl)c4)n3c2)c(C)c1]

General procedure D2 was employed using 4-(3-(3-chlorophenyl)imidazo[1,5-a]pyridin-6-yl)-3-methylbenzoic acid (12 mg, 0.044 mmol, 1 eq.) and N¹,N¹-diethylethane-1,2-diamine (12 μL, 0.088 mmol, 2 eq.). A yellow solid (11 mg, 54%) was isolated by CombiFlash chromatography (C18, ACN, 100%). ¹H NMR (400 MHZ, CDCl₃) δ 8.16 (d, J=0.80 Hz, 1H), 7.82 (t, J=1.8 Hz, 1H), 7.75 (s, 1H), 7.68 (dt, J=7.5, 1.5 Hz, 1H), 7.65-7.59 (m, 2H), 7.56 (dd, J=9.4, 1.1 Hz, 1H), 7.48-7.37 (m, 2H), 7.32 (d, J=7.8 Hz, 1H), 6.95 (s, 1H), 6.77 (dd, J=9.3, 1.4 Hz, 1H), 3.50 (q, J=5.5 Hz, 2H), 2.67 (t, J=6.0 Hz, 2H), 2.58 (q, J=7.1 Hz, 4H), 2.39 (s, 3H), 1.05 (t, J=7.1 Hz, 6H).

### Example 64. 4-(3-(3-chlorophenyl)imidazo[1,5-a]pyridin-6-yl)-N-(2-(diisopropylamino)ethyl)-3-methylbenzamide

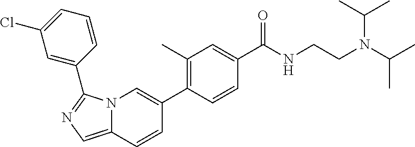 [SMILES: Cc1cc(C(=O)NCCN(C(C)C)C(C)C)ccc1-c1ccc2cnc(-c3cccc(Cl)c3)n2c1]

General procedure D2 was employed using 4-(3-(3-chlorophenyl)imidazo[1,5-a]pyridin-6-yl)-3-methylbenzoic acid (16 mg, 0.044 mmol, 1 eq.) and N¹,N¹-diisopropylethane-1,2-diamine (16 μL, 0.088 mmol, 2 eq.). A yellow solid (10 mg, 46%) was isolated by CombiFlash chromatography (C18, ACN, 100%). ¹H NMR (400 MHZ, CDCl₃) δ 8.16 (q, J=1.1 Hz, 1H), 7.82 (t, J=1.9 Hz, 1H), 7.76 (s, 1H), 7.68 (dt, J=7.5, 1.5 Hz, 1H), 7.65-7.58 (m, 2H), 7.56 (dd, J=9.3, 1.1 Hz, 1H), 7.48-7.36 (m, 2H), 7.31 (d, J=7.9 Hz, 1H), 7.00 (s, 1H), 6.77 (dd, J=9.2, 1.3 Hz, 1H), 3.44 (q, J=5.5 Hz, 2H), 3.08 (p, J=6.6 Hz, 2H), 2.73 (t, J=6.0 Hz, 2H), 2.38 (s, 3H), 1.06 (d, J=6.6 Hz, 12H).

### Example 65. 4-(3-(3-Chlorophenyl)imidazo[1,5-a]pyridin-6-yl)-3-methyl-N-(2-(pyrrolidin-1-yl)ethyl)benzamide

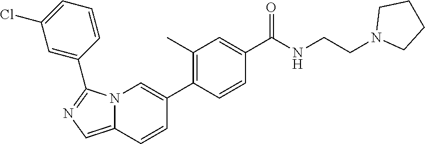 [SMILES: Cc1cc(C(=O)NCCN2CCCC2)ccc1-c1ccc2cnc(-c3cccc(Cl)c3)n2c1]

General procedure D2 was employed using 4-(3-(3-chlorophenyl)imidazo[1,5-a]pyridin-6-yl)-3-methylbenzoic acid (16 mg, 0.044 mmol, 1 eq.) and 2-(pyrrolidin-1-yl) ethan-1-amine (11 μL, 0.088 mmol, 2 eq.). A yellow solid (9 mg, 40%) was isolated by CombiFlash chromatography (C18, ACN, 100%). ¹H NMR (400 MHZ, CDCl₃) δ 8.16 (q, J=1.1 Hz, 1H), 7.82 (t, J=1.8 Hz, 1H), 7.75 (d, J=1.5 Hz, 1H), 7.68 (dt, J=7.5, 1.5 Hz, 1H), 7.66-7.62 (m, 2H), 7.56 (dd, J=9.4, 1.2 Hz, 1H), 7.47-7.37 (m, 2H), 7.32 (d, J=7.9 Hz, 1H), 6.85 (s, 1H), 6.77 (dd, J=9.3, 1.4 Hz, 1H), 3.57 (q, J=5.6 Hz, 2H), 2.73 (t, J=6.0 Hz, 2H), 2.57 (t, J=6.2 Hz, 4H), 2.39 (s, 3H), 1.80 (p, J=3.2 Hz, 4H).

### Example 66. 4-(3-(3-Chlorophenyl)imidazo[1,5-a]pyridin-6-yl)-N-(2-(diethylamino)ethyl)-2-methoxybenzamide

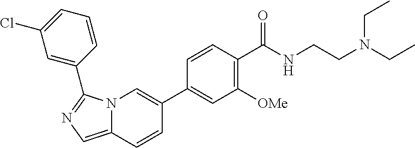 [SMILES: CCN(CC)CCNC(=O)c1ccc(-c2ccc3cnc(-c4cccc(Cl)c4)n3c2)cc1OC]

General procedure D2 was employed using 4-(3-(3-chlorophenyl)imidazo[1,5-a]pyridin-6-yl)-2-methoxybenzoic acid (30 mg, 0.079 mmol, 1 eq.) and N¹,N¹-diethylethane-1,2-diamine (22 μL, 0.16 mmol, 2 eq.). A yellow solid (21 mg, 56%) was isolated by CombiFlash chromatography (C18, ACN, 100%). ¹H NMR (400 MHZ, CDCl₃) δ 8.48-8.40 (m, 2H), 8.30 (d, J=8.1 Hz, 1H), 7.86 (t, J=1.9 Hz, 1H), 7.72 (d, J=7.6 Hz, 1.4 Hz, 1H), 7.64-7.57 (m, 2H), 7.53-7.41 (m, 2H), 7.24 (dd, J=8.1, 1.7 Hz, 1H), 7.08 (d, J=1.7 Hz, 1H), 7.04 (dd, J=9.4, 1.4 Hz, 1H), 4.03 (s, 3H), 3.55 (q, J=5.4 Hz, 2H), 2.66 (t, J=5.9 Hz, 2H), 2.63-2.54 (m, 4H), 1.08 (t, J=7.3 Hz, 6H).

### Example 67. 4-(3-(3-Chlorophenyl)imidazo[1,5-a]pyridin-6-yl)-N-(2-(diisopropylamino)ethyl)-2-methoxybenzamide

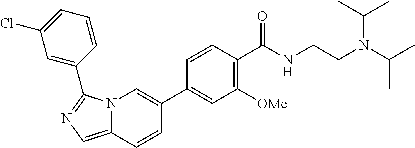 [SMILES: COc1cc(-c2ccc3cnc(-c4cccc(Cl)c4)n3c2)ccc1C(=O)NCCN(C(C)C)C(C)C]

General procedure D2 was employed using 4-(3-(3-chlorophenyl)imidazo[1,5-a]pyridin-6-yl)-2-methoxybenzoic acid (20 mg, 0.053 mmol, 1 eq.) and N¹,N¹-diisopropylethane-1,2-diamine (19 μL, 0.11 mmol, 2 eq.). A yellow solid (9 mg, 30%) was isolated by CombiFlash chromatography (C18, ACN, 100%). ¹H NMR (400 MHZ, CDCl₃) δ 8.42 (q, J=1.2 Hz, 1H), 8.34-8.29 (m, 2H), 7.86 (t, J=1.8 Hz, 1H), 7.72 (dt, J=7.6, 1.5 Hz, 1H), 7.64-7.58 (m, 2H), 7.53-7.42 (m, 2H), 7.24 (dd, J=8.2, 1.6 Hz, 1H), 7.08 (d, J=1.6 Hz, 1H), 7.04 (dd, J=9.4, 1.4 Hz, 2H), 4.02 (s, 3H), 3.51 (q, J=5.7 Hz, 2H), 3.10 (p, J=6.6 Hz, 2H), 2.70 (t, J=6.1 Hz, 2H), 1.05 (d, J=6.7 Hz, 12H).

### Example 68. 4-(3-(3-Chlorophenyl)imidazo[1,5-a]pyridin-6-yl)-2-methoxy-N-(2-(pyrrolidin-1-yl)ethyl)benzamide

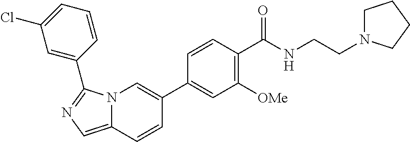 [SMILES: COc1cc(-c2ccc3cnc(-c4cccc(Cl)c4)n3c2)ccc1C(=O)NCCN1CCCC1]

General procedure D2 was employed using 4-(3-(3-chlorophenyl)imidazo[1,5-a]pyridin-6-yl)-2-methoxybenzoic acid (16 mg, 0.042 mmol, 1 eq.) and 2-(pyrrolidin-1-yl) ethan-1-amine (11 μL, 0.084 mmol, 2 eq.). A yellow solid (14 mg, 70%) was isolated by CombiFlash chromatography (C18, ACN, 100%). ¹H NMR (400 MHZ, CDCl₃) δ 8.42 (d, J=0.8 Hz, 1H), 8.39 (t, J=5.2 Hz, 1H), 8.29 (d, J=8.1 Hz, 1H), 7.86 (t, J=1.9 Hz, 1H), 7.72 (dt, J=7.5 Hz, 1.4 Hz, 1H), 7.62-7.59 (m, 2H), 7.52-7.42 (m, 2H), 7.24 (dd, J=8.1 Hz, 1.6 Hz, 1H), 7.10-7.02 (m, 2H), 4.01 (s, 3H), 3.60 (q, J=5.8 Hz, 2H), 2.72 (t, J=6.3 Hz, 2H), 2.59 (t, J=6.2 Hz, 4H), 1.82 (p, J=3.3 Hz, 4H).

### Example 69. N-(2-(Diethylamino)ethyl)-2-isopropyl-4-(3-(4-tolyl)-[1,2,4]triazolo[4,3-b]pyridazin-6-yl)benzamide

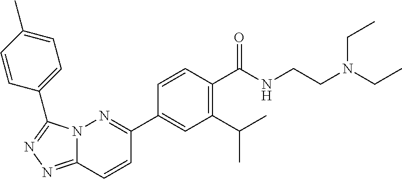 [SMILES: CCN(CC)CCNC(=O)c1ccc(-c2ccc3nnc(-c4ccc(C)cc4)n3n2)cc1C(C)C]

General procedure D5 was employed using 2-isopropyl-4-(3-(4-tolyl)-[1,2,4]triazolo[4,3-6]pyridazin-6-ylbenzoic acid (40 mg, 0.11 mmol, 1 eq.) and N¹, N¹-diethylethane-1,2-diamine (31 μL, 0.21 mmol, 2 eq.). A white solid (37 mg, 73%) was isolated by CombiFlash chromatography (C18, ACN/water 9:1). ¹H NMR (400 MHZ, CDCl₃) δ 8.50 (d, J=8.3 Hz, 2H), 8.25 (d, J=9.7 Hz, 1H), 8.04 (d, J=1.8 Hz, 1H), 7.86 (dd, J=8.0, 1.8 Hz, 1H), 7.71 (d, J=8.0 Hz, 1H), 7.60 (d, J=9.7 Hz, 1H), 7.40 (d, J=8.0 Hz, 2H), 3.93-3.82 (m, 2H), 3.53 (p, J=6.8 Hz, 1H), 3.36-3.00 (m, 6H), 2.47 (s, 3H), 1.50-1.39 (m, 6H), 1.36 (d, J=6.9 Hz, 6H).

### Example 70. 2-Chloro-N-(2-(diethylamino)ethyl)-4-(3-(4-tolyl)-[1,2,4]triazolo[4,3-b]pyridazin-6-yl)benzamide

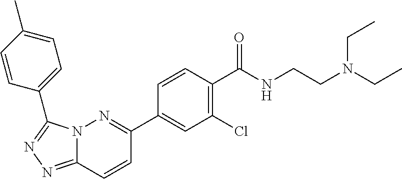 [SMILES: CCN(CC)CCNC(=O)c1ccc(-c2ccc3nnc(-c4ccc(C)cc4)n3n2)cc1Cl]

General procedure C3 was employed using methyl 2-chloro-4-(3-(4-tolyl)-[1,2,4]triazolo[4,3-b]pyridazin-6-yl)benzoate (100 mg, 0.26 mmol, 1 eq.). 2-chloro-4-(3-(4-tolyl)-[1,2,4]triazolo[4,3-b]pyridazin-6-yl)benzoic acid was isolated as yellow solids (77 mg, 80%). Then general procedure D5 was employed using the acid (40 mg, 0.11 mmol) and N¹, N¹-diethylethane-1,2-diamine (32 μL, 0.22 mmol, 2 eq.). A white solid (18 mg, 35%) was isolated by CombiFlash chromatography (C18, ACN/water 9:1). ¹H NMR (400 MHZ, CDCl₃) δ 8.87 (t, J=5.6 Hz, 1H), 8.42 (d, J=8.1 Hz, 2H), 8.35 (d, J=9.7 Hz, 1H), 8.04 (d, J=1.6 Hz, 1H), 7.92 (d, J=1.8 Hz, 1H), 7.72 (d, J=8.0 Hz, 1H), 7.59 (d, J=9.7 Hz, 1H), 7.42 (d, J=7.9 Hz, 2H), 3.91 (q, J=5.3 Hz, 2H), 3.38 (q, J=4.8 Hz, 2H), 3.24 (p, J=3.7 Hz, 4H), 2.48 (s, 3H), 1.41 (t, J=7.3 Hz, 6H).

### Example 71. N-(2-(diisopropylamino)ethyl)-1-(3-(4-tolyl)-[1,2,4]triazolo[4,3-b]pyridazin-6-yl)piperidine-4-carboxamide

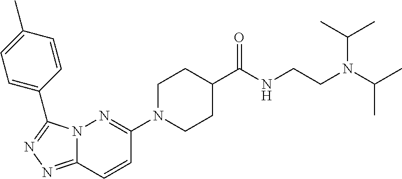 [SMILES: Cc1ccc(-c2nnc3ccc(N4CCC(C(=O)NCCN(C(C)C)C(C)C)CC4)nn23)cc1]

General procedure F was employed using methyl 1-(3-(4-tolyl)-[1,2,4]triazolo[4,3-b]pyridazin-6-yl)piperidine-4-carboxylate (20 mg, 0.057 mmol, 1 eq.) and N¹, N¹-diisopropylethane-1,2-diamine (2 mL). A white solid (13 mg, 50%) was isolated by CombiFlash chromatography (C18, ACN/Water, 1:1). ¹H NMR (400 MHZ, CDCl₃) δ 8.37 (d, J=8.2 Hz, 2H), 7.92 (d, J=10.1 Hz, 1H), 7.34 (d, J=8.1 Hz, 2H), 6.97 (d, J=10.2 Hz, 1H), 6.33 (s, 1H), 4.22 (d, J=13.5 Hz, 2H), 3.24 (q, J=5.5 Hz, 2H), 3.13-2.97 (m, 4H), 2.60 (t, J=6.0 Hz, 2H), 2.46-2.34 (m, 4H), 2.03 (d, J=14.0 Hz, 3.3 Hz, 2H), 1.85 (qd, J=12.5, 4.0 Hz, 2H), 1.01 (d, J=6.6 Hz, 12H).

### Example 72. N-(2-(pyrrolidin-1-yl)ethyl)-1-(3-(4-tolyl)-[1,2,4]triazolo[4,3-b]pyridazin-6-yl)piperidine-4-carboxamide

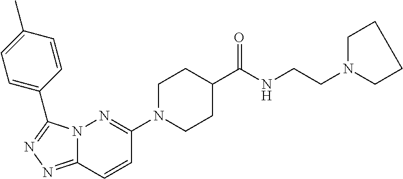 [SMILES: Cc1ccc(-c2nnc3ccc(N4CCC(C(=O)NCCN5CCCC5)CC4)nn23)cc1]

General procedure F was employed using methyl 1-(3-(4-tolyl)-[1,2,4]triazolo[4,3-b]pyridazin-6-yl)piperidine-4-carboxylate (20 mg, 0.057 mmol, 1 eq.) and 2-(1-pyrrolidinyl)ethylamine (2 mL). A white solid (14 mg, 57%) was isolated by CombiFlash chromatography (C18, ACN/Water, 1:1). ¹H NMR (400 MHz, CDCl₃) δ 8.37 (d, J=8.3 Hz, 2H), 7.91 (d, J=10.0 Hz, 1H), 7.34 (d, J=8.0 Hz, 2H), 6.96 (d, J=10.1 Hz, 1H), 6.23 (s, 1H), 4.22 (dt, J=13.4 Hz, 3.3 Hz, 2H), 3.37 (q, J=5.5 Hz, 2H), 3.13-3.03 (m, 2H), 2.61 (t, J=6.0 Hz, 2H), 2.53 (t, J=5.5 Hz, 4H), 2.47-2.36 (m, 4H), 2.01 (dd, J=13.5 Hz, 3.6 Hz, 2H), 1.96-1.85 (m, 2H), 1.80 (p, J=3.3 Hz, 4H).

### Example 73. N-(3-(Diethylamino)propyl)-1-(3-(4-tolyl)-[1,2,4]triazolo[4,3-b]pyridazin-6-yl)azetidine-3-carboxamide

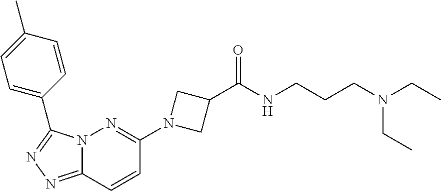 [SMILES: CCN(CC)CCCNC(=O)C1CN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)azetidine-3-carboxylate (25 mg, 0.077 mmol, 1 eq.) and 3-(diethylamino)-1-propanamine (0.8 mL). A white solid (32 mg, 98%) was isolated by CombiFlash chromatography (C18, ACN/Water, 1:1). ¹H NMR (400 MHZ, CDCl₃) δ 8.44-8.34 (m, 3H), 7.90 (d, J=9.8 Hz, 1H), 7.31 (d, J=8.0 Hz, 2H), 6.52 (d, J=9.8 Hz, 1H), 4.39-4.27 (m, 4H), 3.48-3.39 (m, 3H), 2.62-2.48 (m, 6H), 2.43 (s, 3H), 1.68 (p, J=6.0 Hz, 2H), 1.05 (t, J=7.1 Hz, 6H).

### Example 74. N-(2-(Diethylamino)ethyl)-N-methyl-1-(3-(4-tolyl)-[1,2,4]triazolo[4,3-b]pyridazin-6-yl)azetidine-3-carboxamide

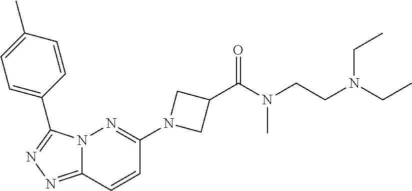 [SMILES: CCN(CC)CCN(C)C(=O)C1CN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)azetidine-3-carboxylate (60 mg, 0.19 mmol, 1 eq.) and 1-(diethylamino)-2-(methylamino) ethane (1.2 mL). A white solid (19 mg, 24%) was isolated by CombiFlash chromatography (C18, ACN/Water, 1:1). ¹H NMR (400 MHZ, CDCl₃) δ 8.39 (dd, J=8.3, 1.9 Hz, 2H), 7.90 (d, J=9.8 Hz, 1H), 7.32 (dd, J=8.3, 3.1 Hz, 2H), 6.53 (d, J=9.8 Hz, 1H), 4.48-4.38 (m, 2H), 4.36-4.26 (m, 2H), 4.02-3.76 (m, 1H), 3.49 (t, J=6.9 Hz, 1H), 3.28 (t, J=6.7 Hz, 1H), 3.01 (d, J=6.9 Hz, 3H), 2.64-2.50 (m, 6H), 2.43 (s, 3H), 1.04 (dt, J=11.7, 7.1 Hz, 6H).

### Example 75. N-(2-(Pyridin-2-yl)ethyl)-1-(3-(4-tolyl)-[1,2,4]triazolo[4,3-b]pyridazin-6-yl)azetidine-3-carboxamide

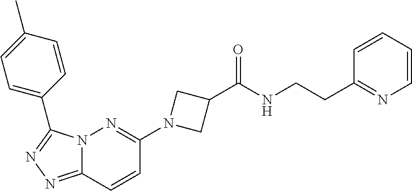 [SMILES: Cc1ccc(-c2nnc3ccc(N4CC(C(=O)NCCc5ccccn5)C4)nn23)cc1]

General procedure F was employed using methyl 1-(3-(4-tolyl)-[1,2,4]triazolo[4,3-b]pyridazin-6-yl)azetidine-3-carboxylate (30 mg, 0.093 mmol, 1 eq.) and 2-(2-pyridyl)ethylamine (0.7 mL). A white solid (33 mg, 86%) was isolated by CombiFlash chromatography (C18, ACN/Water, 1:1). ¹H NMR (400 MHz, CDCl₃) δ 8.52 (d, J=5.0 Hz, 1H), 8.39 (d, J=8.2 Hz, 2H), 7.90 (d, J=9.8 Hz, 1H), 7.66 (t, J=7.7, 1.9 Hz, 1H), 7.32 (d, J=8.0 Hz, 2H), 7.24-7.17 (m, 2H), 7.08 (s, 1H), 6.51 (d, J=9.9 Hz, 1H), 4.37-4.27 (m, 4H), 3.74 (q, J=5.7 Hz, 2H), 3.49 (p, J=7.6 Hz, 1H), 3.04 (t, J=6.1 Hz, 2H), 2.43 (s, 3H).

### Example 76. N-(2-(Pyridin-3-yl)ethyl)-1-(3-(4-tolyl)-[1,2,4]triazolo[4,3-b]pyridazin-6-yl)azetidine-3-carboxamide

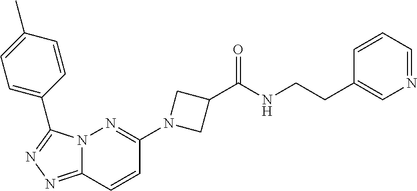 [SMILES: Cc1ccc(-c2nnc3ccc(N4CC(C(=O)NCCc5cccnc5)C4)nn23)cc1]

General procedure F was employed using methyl 1-(3-(4-tolyl)-[1,2,4]triazolo[4,3-b]pyridazin-6-yl)azetidine-3-carboxylate (30 mg, 0.093 mmol, 1 eq.) and 2-(3-pyridyl)ethylamine (0.7 mL). A white solid (27 mg, 70%) was isolated by CombiFlash chromatography (C18, ACN/Water, 1:1). ¹H NMR (400 MHZ, CDCl₃) δ 8.51 (dd, J=4.8, 1.7 Hz, 1H), 8.47 (d, J=2.2 Hz, 1H), 8.37 (dt, J=8.3, 2.2 Hz, 2H), 7.86 (d, J=9.8 Hz, 1H), 7.56 (dt, J=7.8, 1.8 Hz, 1H), 7.32 (d, J=8.0 Hz, 2H), 7.29-7.27 (m, 1H), 6.46 (d, J=9.8 Hz, 1H), 5.91 (t, J=6.2 Hz, 1H), 4.36-4.22 (m, 4H), 3.62 (q, J=6.8 Hz, 2H), 3.48-3.37 (m, 1H), 2.90 (t, J=7.0 Hz, 2H), 2.43 (s, 3H).

### Example 77. N-(2-(Ethyl(2,2,2-trifluoroethyl)amino)ethyl)-1-(3-(4-tolyl)-[1,2,4]triazolo[4,3-b]pyridazin-6-yl)azetidine-3-carboxamide

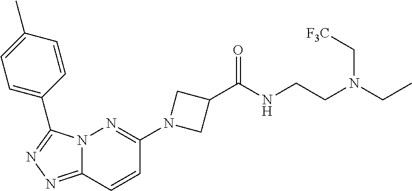 [SMILES: CCN(CCNC(=O)C1CN(c2ccc3nnc(-c4ccc(C)cc4)n3n2)C1)CC(F)(F)F]

General procedure F was employed using methyl 1-(3-(4-tolyl)-[1,2,4]triazolo[4,3-b]pyridazin-6-yl)azetidine-3-carboxylate (30 mg, 0.093 mmol, 1 eq.) and 2-amino-1-[N-ethyl(2,2,2-trifluoroethyl)amino]ethane (0.7 mL). A white solid (35 mg, 82%) was isolated by CombiFlash chromatography (C18, ACN/Water, 1:1). ¹H NMR (400 MHZ, CDCl₃) δ 8.39 (dt, J=8.3, 1.9 Hz, 2H), 7.90 (d, J=9.8 Hz, 1H), 7.31 (d, J=8.0 Hz, 2H), 6.52 (d, J=9.8 Hz, 1H), 6.16 (t, J=5.5 Hz, 1H), 4.40-4.27 (m, 4H), 3.53-3.43 (m, 1H), 3.38 (q, J=5.5 Hz, 2H), 3.07 (q, J=9.4 Hz, 2H), 2.80-2.67 (m, 4H), 2.43 (s, 3H), 1.06 (t, J=7.1 Hz, 3H).

### Example 78. N-(1-(Diethylamino) propan-2-yl)-1-(3-(4-tolyl)-[1,2,4]triazolo[4,3-b]pyridazin-6-yl)azetidine-3-carboxamide

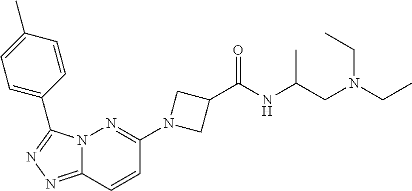 [SMILES: CCN(CC)CC(C)NC(=O)C1CN(c2ccc3nnc(-c4ccc(C)cc4)n3n2)C1]

General procedure F was employed using methyl 1-(3-(4-tolyl)-[1,2,4]triazolo[4,3-b]pyridazin-6-yl)azetidine-3-carboxylate (30 mg, 0.093 mmol, 1 eq.) and 2-amino-1-(diethylamino) propane (0.7 mL). A white solid (8 mg, 20%) was isolated by CombiFlash chromatography (C18, ACN/Water, 1:1). ¹H NMR (400 MHZ, CDCl₃) δ 8.39 (d, J=8.2 Hz, 2H), 7.91 (d, J=9.8 Hz, 1H), 7.31 (d, J=8.1 Hz, 2H), 6.52 (d, J=9.8 Hz, 1H), 6.15 (s, 1H), 4.45-4.25 (m, 4H), 3.96-3.84 (m, 1H), 3.53-3.39 (m, 1H), 2.65-2.33 (m, 9H), 1.23 (d, J=6.3 Hz, 3H), 1.01 (t, J=7.1 Hz, 6H).

### Example 79. N,N-Diethyl-2-(4-(1-(3-(4-tolyl)-[1,2,4]triazolo[4,3-b]pyridazin-6-yl) azetidin-3-yl)-1H-1,2,3-triazol-1-yl) ethan-1-amine

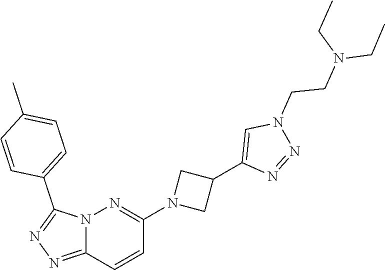 [SMILES: CCN(CC)CCn1cc(C2CN(c3ccc4nnc(-c5ccc(C)cc5)n4n3)C2)nn1]

General procedure E1 was employed using 6-chloro-3-(4-tolyl)-[1,2,4]triazolo[4,3-b]pyridazine (200 mg, 0.82 mmol, 1 eq.) and 3-ethynylazetidine (240 mg, 2.05 mmol, 2.5 eq.). The 6-(3-ethynylazetidin-1-yl)-3-(4-tolyl)-[1,2,4]triazolo[4,3-b]pyridazine was separated as a white solid (0.16 g, 68%) was isolated by CombiFlash chromatography (silica gel, MeOH/CH₂Cl₂, 1:19). The product was carried to the Click reaction without further characterization. To a methanol solution of 2-azido-N,N-diethylethan-1-amine (88 mg, 0.63 mmol, 3 eq.) was added 6-(3-ethynylazetidin-1-yl)-3-(4-tolyl)-[1,2,4]triazolo[4,3-b]pyridazine (60 mg, 0.21 mmol, 1 eq.) and Cu(OAc)₂ (0.4 M in 125 μL H₂O, 0.24 eq.). Then TEA (0.14 mL, 0.10 mmol, 5 eq.) was added 3 to the solution. The mixture was heated at 55° C. for 12 h. The resulting suspension was filtered through a pad of celite and then purified by CombiFlash chromatography (C18, ACN/Water, 9:1). ¹H NMR (400 MHZ, CDCl₃) δ 8.41 (d, J=8.3, 2.1 Hz, 2H), 7.92 (d, J=9.8 Hz, 1H), 7.64 (s, 1H), 7.31 (d, J=8.1 Hz, 2H), 6.56 (d, J=9.8 Hz, 1H), 4.57 (t, J=8.3 Hz, 2H), 4.40 (t, J=6.3 Hz, 2H), 4.35 (dd, J=8.1, 6.2 Hz, 2H), 4.25-4.17 (m, 1H), 2.88 (t, J=6.2 Hz, 2H), 2.55 (q, J=7.1 Hz, 4H), 2.42 (s, 3H), 0.97 (t, J=7.1 Hz, 6H).

### Example 80. Inhibition of Sperm Hyperactivated Motility (HAM) in Mouse

The following outlines the procedure that was used to measure sperm hyperactivated motility (HAM) in mouse.

### A. Isolation of Mouse Sperm.

1) Dissect the epididymis and place it on a solid support. Make 5-6 cuts with a razor blade.
2) Place the cauda epididymis in a 2 ml, round bottom tube, containing 0.5 ml of Whitten's media (non-capacitated medium-see solution section below-) with Ca²⁺ pre-warmed at pH 7.2.
3) Incubate the sample at 37° C. for 10-15 min.
4) Take out the epididymis tissue from the tube and discard.
5) Supernatant contains the swim-up sperm.

### B. Determination of HAM

6) Count sperm number in a hemocytometer.
7) Prepare the tubes for the assay with non capacitated medium +/− different concentrations of the compound to be tested in a final volume of 100 microliters. Add polyvinylpirroldone (PVP) at a final concentration of 0.2%.
8) Add sperm (1,000,000 cells) to each tube and incubate at 37° C. for 1 h.
9) Add 100 microliters of 2× capacitating media and incubate for another 90 min at 37° C.
10) Take aliquot from each tube and add 0.5 ul of the SYTO21 dye. Wait 1′ and load the sample in a chamber and read motility using computer assisted sperm analysis (CASA).

The following solutions were use in the procedure above.

### Whitten's Medium

### a) Non-Capacitated Medium (500 ml)

NaCl 100 mM 2.922 gr
KCl 4.7 mM 0.1752 gr
KH₂PO₄ 1.2 mM 0.0816 gr
MgSO₄ 1.2 mM 0.0722 gr
Glucose 5.5 mM 0.4956 gr
Pyruvic Acid 0.8 mM 4 ml (100 mM stock*)
Lactid Acid (Na Lactate) 4.8 mM0.2685 gr
Hepes 20 mM 2.383 gr
CaCl 1.7 mM 0.0943 gr
   Bring to pH (7.2-7.4) with NaOH
   (*) Pyruvic Acid (100 mM): 1.1004 g×100 ml.

### b) Capacitated Medium (2× Medium, for a Final Volume of 10 ml)

To the media above, add 25 mM bicarbonate (0.042 g). Check that pH is 7.2.
Continue by adding 0.5% Albumin (0.05 gr).

### Polyvinylpirroldone Solution

Prepare a stock solution (2%) in water.

### SYTO21 Solution.

Make a 75 micromolar solution in water.

The following Parameters of sperm motility were measured.

TOTAL MOTILITY™. Represents the combination of all movement performed by sperm.

PROGRESSIVE MOTILITY (PM). Represents the linear progressiveness of sperm.

AMPLITUDE OF LATERAL HEAD DISPLACEMENT (ALH). Represents the magnitude of lateral displacement of the sperm head.

LINEARITY (LIN). Measures the straightness of the path followed by sperm. It is derived from the radio of VSL/VCL multiplied by 100.

STRAIGHT LINE VELOCITY (VSL). Represents the distance between the first and last tracked point in the sperm trajectory divided by the time elapsed.

CURVILINEAR VELOCITY (VCL). Is the sum of the distances between each center of brightness, during each frame, divided by the time elapsed.

AVERAGE PATH VELOCITY (VAP). It is an average of the path followed by sperm (reduces the effect of the lateral head displacement).

AMPLITUDE HEAD DISPLACEMENT (AHL). Shows how much the head displaces from the path.

STRAIGHTNESS (STR). Represents the linearity or straight line followed by sperm.

BEAT CROSS FREQUENCY (BCF). Represents the frequency with which the cell track crosses the cell path in either direction.

HYPERACTIVATION. This is the whiplash movement (“figure 8” movement) that sperm obtains when capacitated. It corresponds to (VCL)>100 μm/s, (LIN)<65%, and (ALH)>7.5 μm.

Data is provided in the following table.

| | | % Inhibition | % Inhibition |
|---|---|---|---|
| Example | Concentration | mouse HAM | human HAM |

| | | | | |
|---|---|---|---|---|
| 1 | 0.1 | μM | 69.4% | 72.5% |
| | 1 | μM | 71.2% | 81.2% |
| | 10 | μM | 74.4% | 89.3% |
| 2 | 0.1 | μM | 74.7% | 78.6% |
| | 1 | μM | 82.1% | 80.2% |
| | 10 | μM | 83.6% | 92.1% |
| 3 | 0.1 | μM | 25.4% | 91.0% |
| | 1 | μM | 27.9% | 95.4% |
| | 10 | μM | 28.0% | 99.1% |
| 4 | 0.1 | μM | 14.3% | 90.0% |
| | 1 | μM | 17.7% | 97.4% |
| | 10 | μM | 17.9% | 99.1% |
| 5 | 0.1 | μM | 15.6% | 86.3% |
| | 1 | μM | 43.9% | 94.3% |
| | 10 | μM | 53.1% | 100.0% |
| 6 | 0.1 | μM | 14.1% | 79.4% |
| | 1 | μM | 13.2% | 79.9% |
| | 10 | μM | 24.2% | 93.1% |
| 7 | 0.1 | μM | 69.1% | 76.8% |
| | 1 | μM | 70.4% | 89.2% |
| | 10 | μM | 75.9% | 97.5% |
| 8 | 0.1 | μM | 41.9% | 62.9% |
| | 1 | μM | 45.4% | 75.3% |
| | 10 | μM | 47.3% | 93.2% |
| 9 | 0.1 | μM | 53.3% | 83.1% |
| | 1 | μM | 52.8% | 94.8% |
| | 10 | μM | 57.4% | 97.8% |
| 10 | 0.1 | μM | 74.4% | 85.3% |
| | 1 | μM | 80.3% | 87.7% |
| | 10 | μM | 84.0% | 95.4% |
| 11 | 0.1 | μM | 20.6% | 81.9% |
| | 1 | μM | 33.4% | 80.9% |
| | 10 | μM | 42.0% | 97.4% |
| 12 | | | | |
| 13 | 0.1 | μM | 39.0% | 85.7% |
| | 1 | μM | 49.4% | 88.7% |
| | 10 | μM | 66.8% | 95.7% |
| 14 | 0.1 | μM | 74.2% | 95.2% |
| | 1 | μM | 79.1% | 97.8% |
| | 10 | μM | 87.1% | 100.0% |
| 15 | 0.1 | μM | 43.0% | 82.6% |
| | 1 | μM | 44.4% | 84.0% |
| | 10 | μM | 54.0% | 86.6% |
| 16 | 0.1 | μM | 67.8% | 63.3% |
| | 1 | μM | 69.1% | 77.0% |
| | 10 | μM | 73.0% | 82.3% |
| 17 | 0.1 | μM | 68.3% | 83.3% |
| | 1 | μM | 76.5% | 85.6% |
| | 10 | μM | 82.5% | 90.7% |
| 18 | 0.1 | μM | 83.0% | 61.9% |
| | 1 | μM | 83.5% | 47.8% |
| | 10 | μM | 87.5% | 80.9% |
| 19 | 0.1 | μM | 55.4% | 84.8% |
| | 1 | μM | 66.0% | 84.9% |
| | 10 | μM | 78.4% | 94.2% |
| 20 | 0.1 | μM | 77.5% | 73.0% |
| | 1 | μM | 84.7% | 75.2% |
| | 10 | μM | 91.1% | 91.6% |
| 21 | 0.1 | μM | 87.3% | 88.1% |
| | 1 | μM | 88.2% | 87.9% |
| | 10 | μM | 88.3% | 92.4% |
| 22 | 0.1 | μM | 66.2% | 72.1% |
| | 1 | μM | 82.8% | 79.2% |
| | 10 | μM | 88.6% | 82.9% |
| 23 | 0.1 | μM | 63.4% | 67.0% |
| | 1 | μM | 83.6% | 70.0% |
| | 10 | μM | 88.0% | 83.5% |
| 24 | 0.1 | μM | 73.9% | 86.4% |
| | 1 | μM | 82.3% | 87.5% |
| | 10 | μM | 91.6% | 93.6% |
| 25 | 0.1 | μM | 58.6% | 87.4% |
| | 1 | μM | 63.0% | 91.9% |
| | 10 | μM | 66.7% | 91.4% |
| 26 | 0.1 | μM | 65.9% | 88.4% |
| | 1 | μM | 73.2% | 94.8% |
| | 10 | μM | 77.4% | 96.0% |
| 27 | 0.1 | μM | 71.5% | 86.9% |
| | 1 | μM | 78.0% | 93.8% |
| | 10 | μM | 86.0% | 96.7% |
| 28 | 0.1 | μM | 68.3% | |
| | 1 | μM | 72.9% | |
| | 10 | μM | 80.1% | |
| 29 | 0.1 | μM | 69.0% | |
| | 1 | μM | 78.0% | |
| | 10 | μM | 83.5% | |
| 30 | 0.1 | μM | 45.1% | |
| | 1 | μM | 54.7% | |
| | 10 | μM | 57.5% | |
| 31 | 0.1 | μM | 85.9% | 87.6% |
| | 1 | μM | 95.5% | 90.3% |
| | 10 | μM | 98.3% | 91.7% |
| 32 | 0.1 | μM | 82.6% | 100.0% |
| | 1 | μM | 84.4% | 100.0% |
| | 10 | μM | 94.2% | 100.0% |
| 33 | 0.1 | μM | 62.3% | |
| | 1 | μM | 63.8% | |
| | 10 | μM | 62.8% | |
| 34 | 0.1 | μM | 72.0% | |
| | 1 | μM | 69.5% | |
| | 10 | μM | 80.8% | |
| 35 | 0.1 | μM | 73.8% | |
| | 1 | μM | 76.3% | |
| | 10 | μM | 75.8% | |
| 36 | 0.1 | μM | 51.9% | |
| | 1 | μM | 53.4% | |
| | 10 | μM | 55.8% | |
| 37 | 0.1 | μM | 49.4% | 68.0% |
| | 1 | μM | 55.8% | 80.2% |
| | 10 | μM | 64.1% | 82.2% |
| 38 | 0.1 | μM | 88.5% | 66.3% |
| | 1 | μM | 91.8% | 78.6% |
| | 10 | μM | 96.6% | 92.8% |
| 39 | 0.1 | μM | 70.4% | |
| | 1 | μM | 79.4% | |
| | 10 | μM | 83.0% | |
| 40 | 0.1 | μM | 91.0% | 78.0% |
| | 1 | μM | 92.4% | 83.8% |
| | 10 | μM | 92.1% | 91.8% |
| 41 | 0.1 | μM | 89.1% | 72.0% |
| | 1 | μM | 93.2% | 65.4% |
| | 10 | μM | 93.7% | 77.2% |
| 42 | 0.1 | μM | 79.5% | 78.7% |
| | 1 | μM | 82.9% | 85.1% |
| | 10 | μM | 93.0% | 93.1% |
| 43 | 0.1 | μM | 65.7% | 64.8% |
| | 1 | μM | 74.9% | 81.4% |
| | 10 | μM | 81.2% | 84.1% |
| 44 | 0.1 | μM | 71.5% | 69.7% |
| | 1 | μM | 75.8% | 77.7% |
| | 10 | μM | 85.1% | 89.3% |
| 45 | 0.1 | μM | 62.5% | 66.3% |
| | 1 | μM | 60.9% | 83.3% |
| | 10 | μM | 60.0% | 91.3% |
| 46 | 0.1 | μM | 69.1% | 66.3% |
| | 1 | μM | 81.9% | 77.3% |
| | 10 | μM | 83.2% | 95.1% |
| 47 | 0.1 | μM | 29.0% | 76.8% |
| | 1 | μM | 33.5% | 84.9% |
| | 10 | μM | 44.1% | 91.2% |
| 48 | 0.1 | μM | 40.0% | 79.2% |
| | 1 | μM | 48.0% | 80.1% |
| | 10 | μM | 53.5% | 96.0% |
| 49 | 0.1 | μM | 47.0% | 96.4% |
| | 1 | μM | 51.7% | 100.0% |
| | 10 | μM | 62.0% | 100.0% |
| 50 | 0.1 | μM | 63.2% | 91.0% |
| | 1 | μM | 70.9% | 96.0% |
| | 10 | μM | 78.3% | 98.6% |
| 51 | 0.1 | μM | 51.1% | 72.7% |
| | 1 | μM | 50.6% | 87.8% |
| | 10 | μM | 79.6% | 100.0% |
| 52 | 0.1 | μM | 61.9% | 91.0% |
| | 1 | μM | 73.9% | 92.6% |
| | 10 | μM | 67.8% | 100% |
| 53 | 0.1 | μM | 66.2% | 94.1% |
| | 1 | μM | 64.8% | 95.1% |
| | 10 | μM | 84.5% | 100.0% |
| 54 | 0.1 | μM | | 79.20% |
| | 1 | μM | | 75.70% |
| | 10 | μM | | 94.10% |
| 55 | 0.1 | μM | 55.2% | 88.0% |
| | 1 | μM | 57.4% | 89.1% |
| | 10 | μM | 60.0% | 98.2% |
| 56 | 0.1 | μM | 58.6% | 86.8% |
| | 1 | μM | 59.0% | 84.8% |
| | 10 | μM | 82.8% | 88.9% |
| 57 | 0.1 | μM | 65.6% | 96.3% |
| | 1 | μM | 67.2% | 99.3% |
| | 10 | μM | 83.7% | 100.0% |
| 58 | 0.1 | μM | 63.7% | 39.7% |
| | 1 | μM | 59.9% | 44.7% |
| | 10 | μM | 68.2% | 82.5% |
| 59 | 0.1 | μM | 60.1% | 53.5% |
| | 1 | μM | 66.1% | 62.2% |
| | 10 | μM | 78.4% | 81.6% |
| 60 | 0.1 | μM | 70.7% | 74.3% |
| | 1 | μM | 67.8% | 80.5% |
| | 10 | μM | 85.2% | 98.7% |
| 61 | 0.1 | μM | 74.4% | 85.3% |
| | 1 | μM | 80.4% | 89.0% |
| | 10 | μM | 90.3% | 97.6% |
| 62 | 0.1 | μM | | 78.7% |
| | 1 | μM | | 87.8% |
| | 10 | μM | | 92.7% |
| 63 | 0.1 | μM | 54.9% | 87.1% |
| | 1 | μM | 57.1% | 87.1% |
| | 10 | μM | 67.4% | 93.3% |
| 64 | 0.1 | μM | 59.0% | 82.1% |
| | 1 | μM | 59.9% | 90.5% |
| | 10 | μM | 61.9% | 87.0% |
| 65 | 0.1 | μM | 64.1% | 87.9% |
| | 1 | μM | 68.5% | 88.3% |
| | 10 | μM | 75.3% | 93.6% |
| 66 | 0.1 | μM | 62.7% | 78% |
| | 1 | μM | 61.2% | 85% |
| | 10 | μM | 64.1% | 93% |
| 67 | 0.1 | μM | 57.8% | 89.5% |
| | 1 | μM | 65.2% | 88.8% |
| | 10 | μM | 67.8% | 99.4% |
| 68 | 0.1 | μM | 69.7% | 92.8% |
| | 1 | μM | 86.8% | 98.5% |
| | 10 | μM | 85.9% | 98.5% |
| 69 | 0.1 | μM | | 56.9% |
| | 1 | μM | | 81.2% |
| | 10 | μM | | 93.4% |
| 70 | 0.1 | μM | | 82.6% |
| | 1 | μM | | 82.9% |
| | 10 | μM | | 90.3% |
| 71 | 0.1 | μM | 19.0% | 72.9% |
| | 1 | μM | 27.5% | 75.0% |
| | 10 | μM | 44.6% | 97.2% |
| 72 | 0.1 | μM | 22.3% | 89.8% |
| | 1 | μM | 25.3% | 93.2% |
| | 10 | μM | 44.4% | 100.0% |
| 73 | 0.1 | μM | 65.8% | 77.2% |
| | 1 | μM | 77.6% | 82.0% |
| | 10 | μM | 78.5% | 90.6% |
| 74 | 0.1 | μM | 71.2% | 67.9% |
| | 1 | μM | 75.9% | 91.2% |
| | 10 | μM | 77.5% | 99.4% |
| 75 | 0.1 | μM | 43.5% | 74.5% |
| | 1 | μM | 48.7% | 79.7% |
| | 10 | μM | 49.7% | 85.4% |
| 76 | 0.1 | μM | | 58.4% |
| | 1 | μM | | 76.1% |
| | 10 | μM | | 81.4% |
| 77 | 0.1 | μM | 33.6% | 68.7% |
| | 1 | μM | 51.4% | 74.0% |
| | 10 | μM | 53.0% | 95.5% |
| 78 | 0.1 | μM | | 67.0% |
| | 1 | μM | | 69.6% |
| | 10 | μM | | 85.6% |
| 79 | 0.1 | μM | 42.3% | |
| | 1 | μM | 42.0% | |
| | 10 | μM | 51.4% | |

### Example 81. Inhibition of Sperm Hyperactivated Motility (HAM) in Human

The following outlines the procedure that was used to measure sperm hyperactivated motility (HAM) in human.

### A. Sperm Collection.

1. Obtain a semen sample from a donor.
2. Allow sample to liquefy at 37° C. for 30 min.
3. Place the sample in a 15 mL tube and add an equal volume of F-10 medium with bicarbonate (Sigma Cat #N6013-500) adjusted at a pH of 7.2 and warmed at 37° C.
4. Centrifuge the sample at 330×g for 5 min.
5. Discard supernatant and to the pellet add 750 microliters of F10 medium, slowly and gently along the side of the tube.
6. Let sperm swim up at 37° C. under a 5% CO² atmosphere placing the tube at a 45° angle for 45 min.
7. Collect the swim-up sperm and transfer to a new round base Eppendorf tube.
8. Count the sperm number using a hemocytometer.

### B. Determination of HAM

9. Prepare the tubes for the assay with F10 medium +/− different concentrations of the compound to be tested in a final volume of 100 microliters. Add polyvinylpirroldone (PVP) at a final concentration of 0.2%.
10. Add sperm (1,000,000 cells) to each tube and incubate at 37° C. for 1 h.
11. Add 100 microliters of F10 medium with the addition of 20 mg×ml of bovine serum albumin (BSA), 0.003% sodium pyruvate, 0.36% sodium lactate and 0.2% sodium bicarbonate.
12. Incubate sample for another 3 h at 37° C.
13. Take aliquot from each tube and load the sample in a chamber and read motility using computer assisted sperm analysis (CASA).

### Example 82

The following illustrate representative pharmaceutical dosage forms, containing a compound of formula (I) (‘Compound X’), for therapeutic or prophylactic use in humans.

| | mg/tablet |
|---|---|
| (i) Tablet 1 | |
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |
| | mg/ml |
| (iv) Injection 1 (1 mg/ml) | |
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/ml) | |
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |
| (vi) Aerosol | mg/can |
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 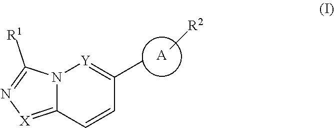
or a salt thereof, wherein:
R¹ is a 6-10 membered aryl, (C₃-C₆)cycloalkyl, or a 5-10 membered heteroaryl, which 6-10 membered aryl, (C₃-C₆)cycloalkyl, and 5-10 membered heteroaryl is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
X is N or CH;
Y is N or CH;
ring A is phenyl, 3-8 membered heterocyclic ring, or a 5-10 membered heteroaryl, which phenyl, 3-8 membered heterocyclic ring and 5-10 membered heteroaryl is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
R² is —C(═O)—NR^{a}R^{b} or a 5-membered heteroaryl ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy;
R^{a} is H or (C₁-C₆)alkyl; and R^{b} is H, (C₁-C₆)alkyl, (C₃-C₆)cycloalkyl, or (C₃-C₆)cycloalkyl(C₁-C₆)alkyl, wherein any (C₁-C₆)alkyl, (C₃-C₆)cycloalkyl, and (C₃-C₆)cycloalkyl(C₁-C₆)alkyl is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, aryl, 5-10 membered heteroaryl, NR^{e}R^{f}, and (C₂-C₆)alkanoyloxy; or
R^{a} and R^{b} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy;
each R^{c} and R^{d} is independently selected from the group consisting of H, (C₁-C₆)alkyl, (C₃-C₆)cycloalkyl, and (C₃-C₆)cycloalkyl(C₁-C₆)alkyl, or R^{c} and R^{d} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy; and
each R^{e} and R^{f} is independently selected from the group consisting of H, (C₁-C₆)alkyl, (C₃-C₆)cycloalkyl, and (C₃-C₆)cycloalkyl(C₁-C₆)alkyl, wherein (C₁-C₆)alkyl, (C₃-C₆)cycloalkyl, and (C₃-C₆)cycloalkyl(C₁-C₆)alkyl are optionally substituted with one or more halo, or R^{e} and R^{f} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy.

2. The compound of claim 1 or a salt thereof, wherein:
R¹ is a 6-10 membered aryl, (C₃-C₆)cycloalkyl, or a 5-10 membered heteroaryl, which 6-10 membered aryl, (C₃-C₆)cycloalkyl, and 5-10 membered heteroaryl is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
X is N or CH;
Y is N or CH;
ring A is phenyl or a 3-8 membered heterocyclic ring, which phenyl and 3-8 membered heterocyclic ring is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy;
R² is —C(═O)—NR^{a}R^{b} or a 5-membered heteroaryl ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy;
R^{a} is H or (C₁-C₆)alkyl; and R^{b} is H, (C₁-C₆)alkyl, (C₃-C₆)cycloalkyl, or (C₃-C₆)cycloalkyl(C₁-C₆)alkyl, wherein any (C₁-C₆)alkyl, (C₃-C₆)cycloalkyl, and (C₃-C₆)cycloalkyl(C₁-C₆)alkyl is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, aryl, NR^{e}R^{f}, and (C₂-C₆)alkanoyloxy; or
R^{a} and R^{b} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy;
each R^{c} and R^{d} is independently selected from the group consisting of H, (C₁-C₆)alkyl, (C₃-C₆)cycloalkyl, and (C₃-C₆)cycloalkyl(C₁-C₆)alkyl, or R^{c} and R^{d} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy; and
each R^{e} and R^{f} is independently selected from the group consisting of H, (C₁-C₆)alkyl, (C₃-C₆)cycloalkyl, and (C₃-C₆)cycloalkyl(C₁-C₆)alkyl, or R^{e} and R^{f} together with the nitrogen to which they are attached form a 3-8 membered heterocyclic ring that is optionally substituted with one or more groups independently selected from the group consisting of halo, (C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy.

3. The compound or salt of claim 1, wherein ring A is phenyl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

4. The compound or salt of claim 1, wherein ring A is a 3-8 membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

5. The compound or salt of claim 1, wherein ring A is a 5-10 membered heteroaryl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

6. The compound or salt of claim 1, which is a compound of formula (Ia): 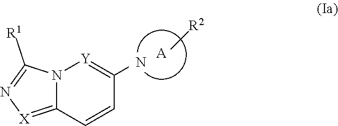
or a salt thereof:
wherein ring A is a 4-, 5-, or 6-membered heterocyclic ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

7. The compound or salt of claim 1, wherein ring A is:

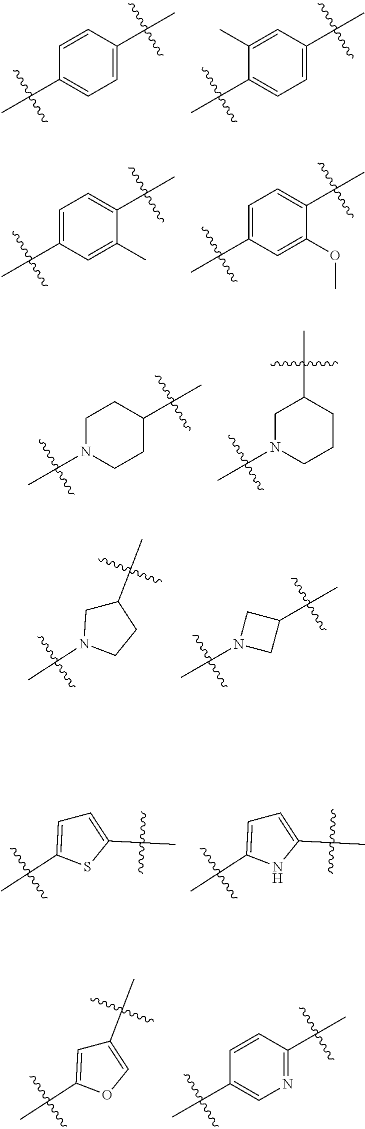

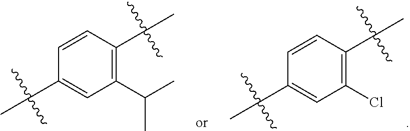 [SMILES: *c1ccc(*)c(C(C)C)c1 ; *c1ccc(*)c(Cl)c1]

8. The compound or salt of claim 1, wherein R¹ is a 6-10 membered aryl, that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

9. The compound or salt of claim 1, wherein R¹ is a (C₃-C₆)cycloalkyl that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, and (C₂-C₆)alkanoyloxy.

10. The compound or salt of claim 1, wherein R¹ is 4-methylphenyl, 3-methylphenyl, 2-methylphenyl, 4-fluorophenyl, 4-methoxyphenyl, 4-trifluoromethylphenyl, 4-chlorophenyl, 3,4-dichlorophenyl, cyclohexyl, naphthyl, 3-trifluoromethylphenyl, 3-methoxyphenyl, or 3-chlorophenyl.

11. The compound or salt of claim 1, wherein X is N and Y is N or X is CH and Y is CH.

12. The compound or salt of claim 1, wherein R² is —C(═O)—NR^{a}R^{b}.

13. The compound or salt of claim 1, wherein R² is a 5-membered heteroaryl ring that is optionally substituted with one or more groups independently selected from the group consisting of halo, hydroxy, carboxy, nitro, (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy, wherein any (C₁-C₆)alkyl, (C₃-C₆)cycloalkyl, (C₃-C₆)cycloalkyl(C₁-C₆)alkyl, (C₁-C₆)alkoxy, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, hydroxy, carboxy, nitro, (C₁-C₆)alkoxy, (C₁-C₆)alkanoyl, (C₁-C₆)alkoxycarbonyl, (C₁-C₆)alkylthio, NR^{c}R^{d}, and (C₂-C₆)alkanoyloxy.

14. The compound or salt of claim 1, wherein R² is:

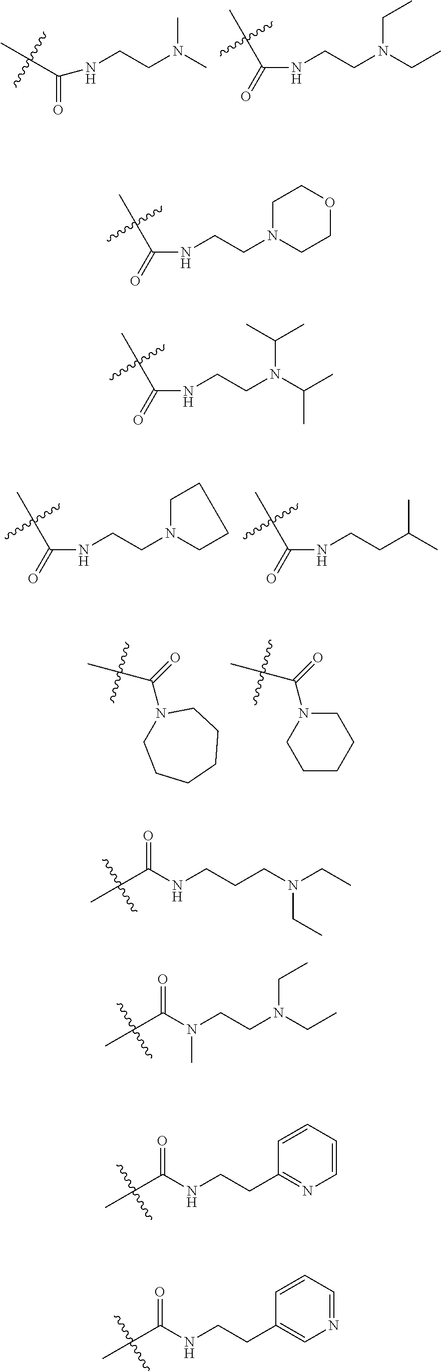

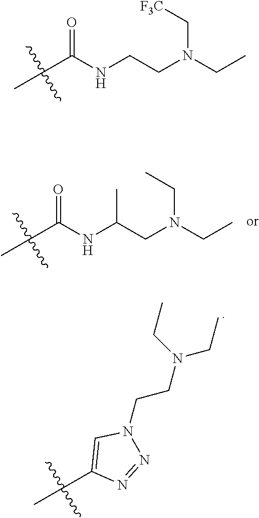 [SMILES: *C(=O)NCCN(CC)CC(F)(F)F ; *C(=O)NC(C)CN(CC)CC ; "or" ; *c1cn(CCN(CC)CC)nn1 ; "."]

15. The compound or salt of claim 1 selected from the group consisting of: 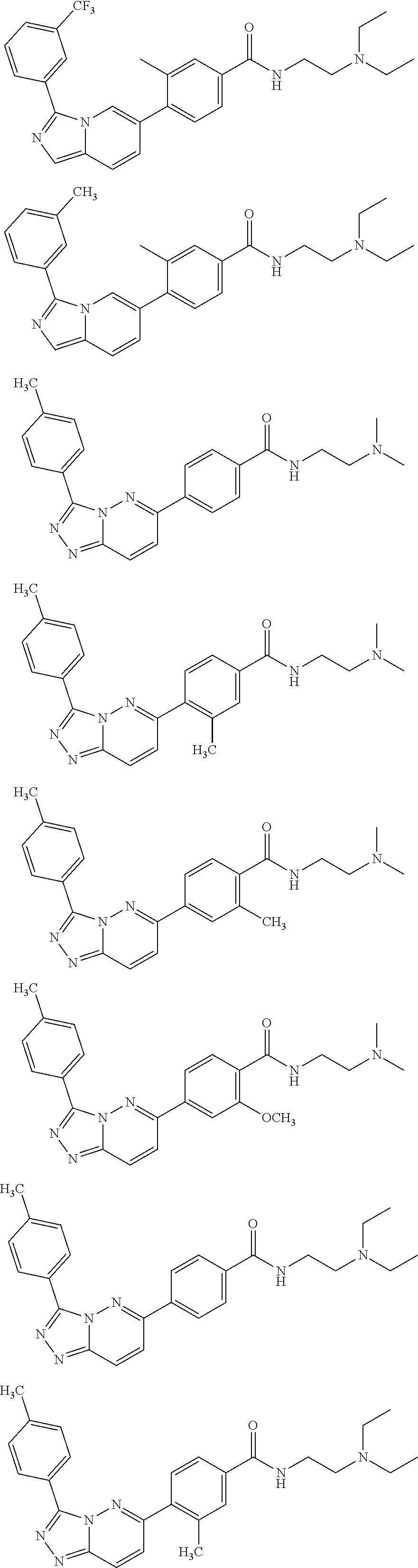 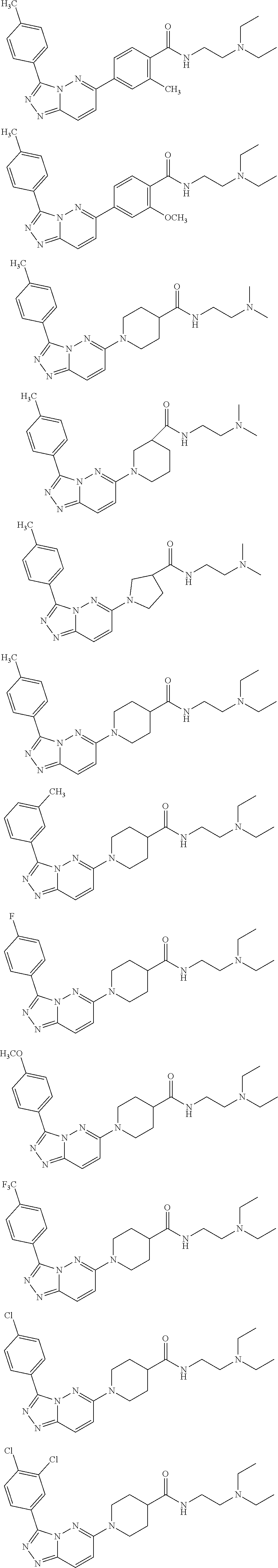 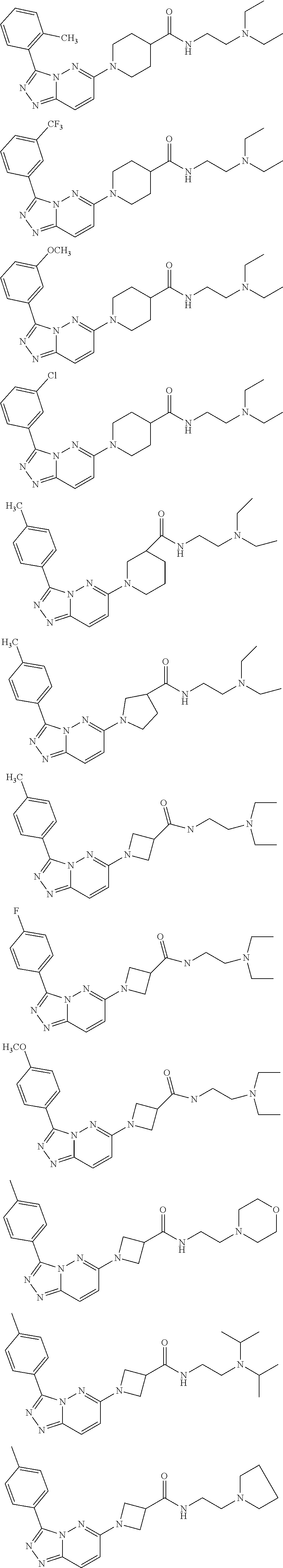 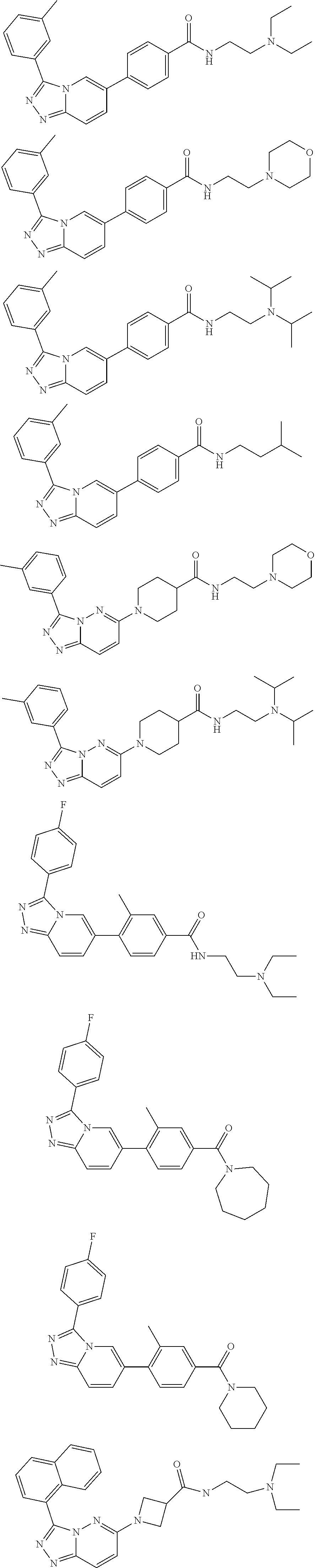 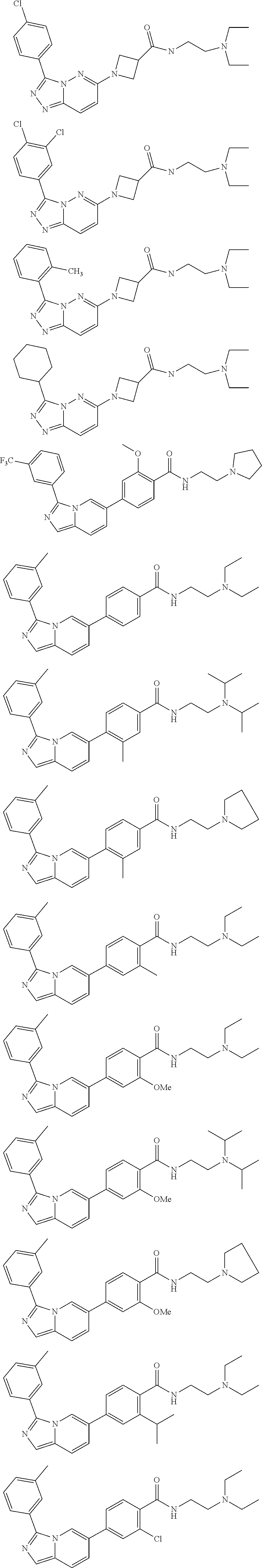 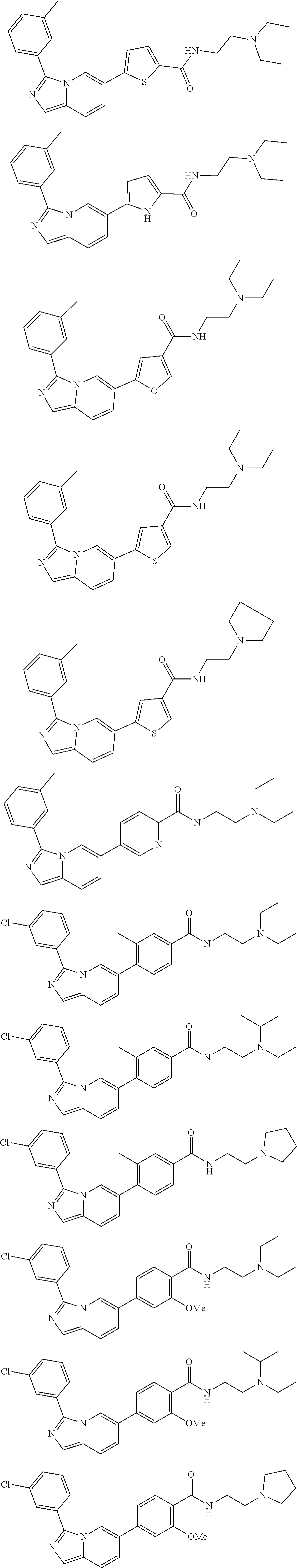 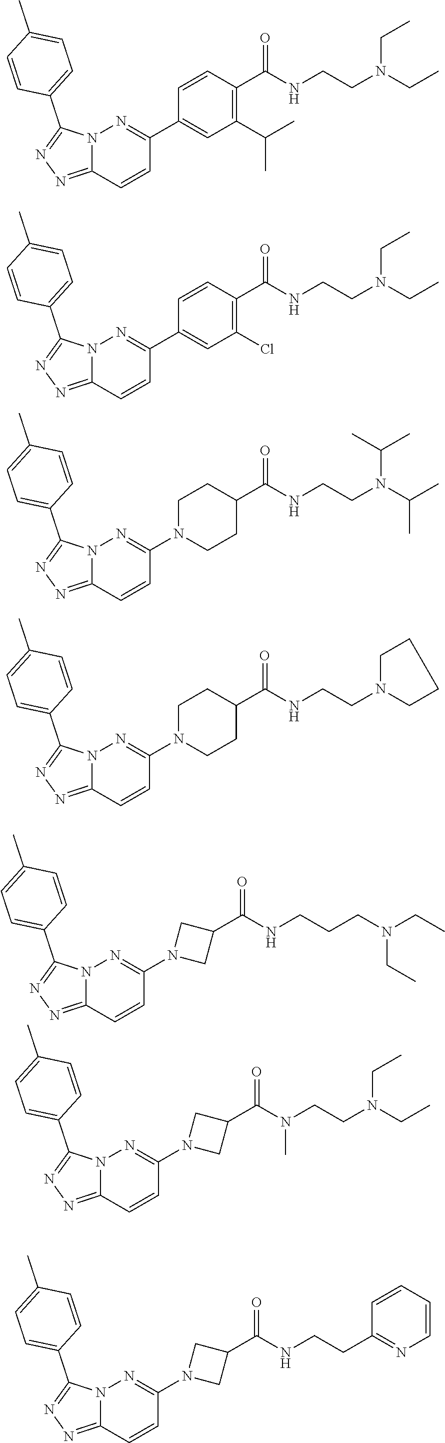 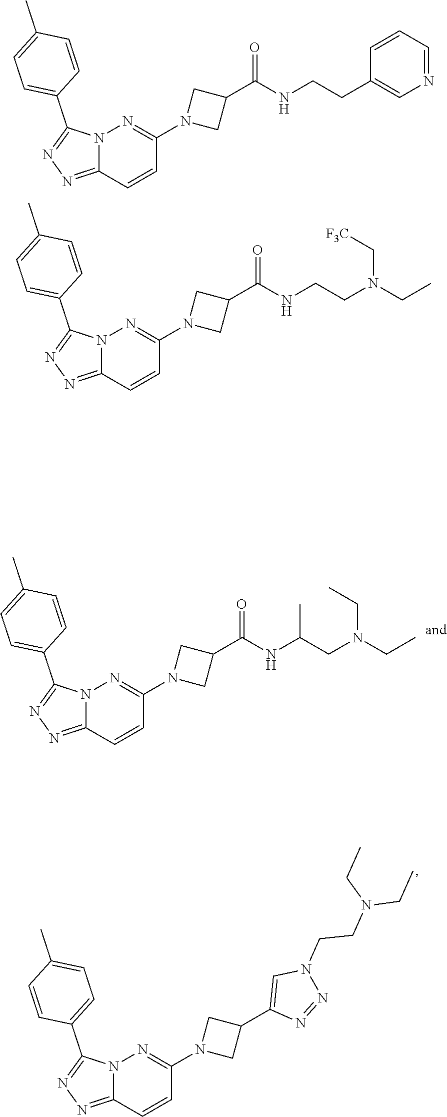
and salts thereof.

16. A pharmaceutical composition comprising a compound or pharmaceutically acceptable salt as described in claim 1, and a pharmaceutically acceptable excipient.

17. A method to reduce the likelihood of conception following intercourse between a male subject and a female subject, comprising administering a compound or pharmaceutically acceptable salt as described in claim 1 to the male subject or to the female subject.

18. A method to reduce sperm motility in a male subject, comprising administering a compound or pharmaceutically acceptable salt as described in claim 1 to the male subject.

19. A method to reduce sperm motility following intercourse between a male subject and a female subject, comprising administering a compound or pharmaceutically acceptable salt as described in claim 1 to the female subject.

20. A method to produce reversible infertility in a male subject, comprising administering a compound or pharmaceutically acceptable salt as described in claim 1 to the male subject.
